SEQUENCE LISTING (15 sequences)

SEQ ID NO: 1 - DNA, 18 bp, Artificial Sequence
  Description of Artificial Sequence Synthetic oligonucleotide
tctcccagcgtgcgccat

SEQ ID NO: 2 - DNA, 23 bp, Artificial Sequence
  Description of Artificial Sequence Synthetic primer
ccctgtggatgactgagtacctg

SEQ ID NO: 3 - DNA, 19 bp, Artificial Sequence
  Description of Artificial Sequence Synthetic primer
ccagcctccgttatcctgg

SEQ ID NO: 4 - DNA, 20 bp, Artificial Sequence
  Description of Artificial Sequence Synthetic probe
ccggcacctgcacacctgga

SEQ ID NO: 5 - DNA, 18 bp, Artificial Sequence
  Description of Artificial Sequence Synthetic oligonucleotide
tctcccagcatgtgccat

SEQ ID NO: 6 - DNA, 18 bp, Artificial Sequence
  Description of Artificial Sequence Synthetic oligonucleotide
taccgcgtgcgaccctct

SEQ ID NO: 7 - DNA, 18 bp, Artificial Sequence
  Description of Artificial Sequence Synthetic oligonucleotide
taccgcgtgcgaccctct

SEQ ID NO: 8 - DNA, 20 bp, Artificial Sequence
  Description of Artificial Sequence Synthetic oligonucleotide
ggctaaatcgctccaccaag

SEQ ID NO: 9 - DNA, 20 bp, Artificial Sequence
  Description of Artificial Sequence Synthetic oligonucleotide
acgcactcagctagtgacac

SEQ ID NO: 10 - DNA, 20 bp, Artificial Sequence
  Description of Artificial Sequence Synthetic primer
tccctggagaagagctacga

SEQ ID NO: 11 - DNA, 20 bp, Artificial Sequence
  Description of Artificial Sequence Synthetic primer
agcactgtgttggcgtacag

SEQ ID NO: 12 - DNA, 20 bp, Artificial Sequence
  Description of Artificial Sequence Synthetic primer
aacaaggtcgtgtccgcaaa

SEQ ID NO: 13 - DNA, 20 bp, Artificial Sequence
  Description of Artificial Sequence Synthetic primer
catctttgctggtgtactcc

SEQ ID NO: 14 - DNA, 18 bp, Homo sapiens
atggcgcacgctgggaga

SEQ ID NO: 15 - DNA, 18 bp, Mus sp.
atggcgcaagccgggaga

(19) United States
(12) Patent Application Publication
(10) Pub. No.: US 2011/0038941 A1
(43) Pub. Date: Feb. 17, 2011

(54) Lipid Nanoparticle Compositions and Methods of Making and Using the Same

(75) Inventors: Robert J. Lee, Columbus, OH (US); Bo Yu, Columbus, OH (US); L. James Lee, Columbus, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/810,900

(22) PCT Filed: Dec. 23, 2008
(86) PCT No.: PCT/US08/88168

§ 371 Date: Aug. 23, 2010

Related U.S. Application Data
(60) Provisional application No. 61/009,268, filed on Dec. 27, 2007.

(51) Int. Cl. A61K 9/00; A61K 31/7088; B01J 19/00; A61K 39/395; A61P 35/00; B32B 37/16

(52) U.S. Cl. 424/498; 514/44 R; 422/129; 514/44 A; 424/133.1; 156/73.1

(57) ABSTRACT

Oligonucleotide-lipid nanoparticles made of at least one oligonucleotide, at least one lipid and at least one complexation agent for the oligonucleotide, methods of making and using, and devices for making the same are disclosed.

### CROSS-REFERENCE TO RELATED APPLICATIONS

This application claims the benefit of U.S. Provisional Application No. 61/009,268 filed Dec. 27, 2007, the disclosure of which is incorporated herein by reference.

### STATEMENT REGARDING FEDERALLY SPONSORED RESEARCH

This invention was made with Government support and the Government has rights in this invention under the grant under the National Science Foundation Grant NSEC (EEC-0425626) Sponsored Research Project Number 60003575.

### TECHNICAL FIELD AND INDUSTRIAL APPLICABILITY OF THE INVENTION

This invention is directed to certain novel compounds, methods for producing them and methods for treating or ameliorating various diseases by using the lipid nanoparticles as drug delivery devices. More particularly, this invention is directed to oligonucleotide-lipid nanoparticles, methods for producing such compounds and methods for treating or ameliorating various diseases using such compounds.

### BACKGROUND OF THE INVENTION

Oligonucleotides, such as antisense deoxyribonucleotides (ODNs), micro RNAs (miRNAs), CpG ODNs, and small interfering RNAs (siRNAs), have shown considerable promise for therapeutic applications. However, these agents have relatively high molecular weights and charge densities, which renders them impermeable to the cellular membrane. In fact, in vitro biological activities of these oligonucleotides require the aid of transfection agents, such as Oligofectamine™ from Invitrogen, in order to be effective. Although free antisense deoxyribonucleotides are being studied in current clinical trials and have shown some efficacy against several types of cancer, there is still a need to further enhance their activity. There is a particular need to enhance the effective delivery of the antisense deoxyribonucleotides to the desired target sites with tissue specificity.

One area of concern is that unmodified oligonucleotides are rapidly degraded by nucleases in the body. Although various chemical modifications, such as a phosphorothioate backbone, have been used to increase the stability of the oligonucleotides, they still suffer from short circulation time due to binding to serum proteins and degradation by serum nucleases.

Other research has involved protamine sulfate, which is a polycation where antisense deoxyribonucleotides-protamine electrostatic complexes have been evaluated for in vivo delivery. However, these complexes lack sufficient colloidal stability and tend to aggregate over time, thereby limiting their usefulness.

Still other research has involved cationic liposomes which have been used to complex and encapsulate oligonucleotides. However, these complexes also lack sufficient colloidal stability, tend to increase in size over time, and are not very stable in the presence of serum, again thereby limiting their usefulness.

An improvement is therefore needed for an oligonucleotide formulation to make such formulation suitable for systemic in vivo administration without the above-described drawbacks.

There is also a need for therapeutic strategies based on the effective delivery of oligonucleotide compositions.

### SUMMARY OF THE INVENTION

In one aspect, there is provided herein an oligonucleotide-lipid nanoparticle comprising at least one oligonucleotide, at least one lipid and at least one complexation agent for the oligonucleotide. In certain embodiments, the oligonucleotide-lipid nanoparticle further includes at least one targeting ligand and/or at least one additional functional component.

In another aspect, there is provided a method for protecting an oligonucleotide from degradation by nucleases and prolonging systemic circulation time in vivo. The method includes loading an oligonucleotide into a lipid nanoparticle, whereby the oligonucleotide-lipid nanoparticle is formed. The in vivo circulation time is further extended by grafting one or more PEG polymers onto the surface of the oligonucleotide-lipid nanoparticle through incorporation of PEG-grafted lipids.

The method can include a solvent removal step which can be accomplished by using a tangential-flow diafiltration method to exchange the nanoparticles into an aqueous buffer and to adjust the oligonucleotide-lipid nanoparticles to a desired concentration.

Various objects and advantages of this invention will become apparent to those skilled in the art from the following detailed description of the preferred embodiment, when read in light of the accompanying drawings.

### BRIEF DESCRIPTION OF THE FIGURES

The patent or application file contains at least one drawing executed in color. Copies of this patent or patent application publication with color drawing(s) will be provided by the Office upon request and payment of the necessary fee.

It is to be understood that various abbreviations used in the Figures, Specification, Examples and Claims can be used interchangeably: lipid nanoparticles are variously designated as “LN”, “LNP”, “LP”, “LPN”, and “lipopolyplex”; oligodeoxynucleotides are variously designated as “ODN”, “ON” and “oligonucleotides”; immunolipid nanoparaticles are varisously designated as “ILN”, “INP” and “IP.”

FIG. 1: Schematic illustration of an oligonucleotide-lipid nanoparticle.

FIG. 2A: Photograph showing K562 chronic myeloid leukemia cells treated with transferrin oligonucleotide-lipid nanoparticles.

FIG. 2B: Photograph showing K562 cells treated with free oligonucleotides.

FIG. 3A: Graph showing the relative cell viability following treatment with a control and with oligonucleotide-lipid nanoparticle formulations.

FIG. 3B: Graph showing the stability of the particle size (nm) of the oligonucleotide-lipid nanoparticles over time.

FIG. 3C: Graphs showing the slow plasma clearance kinetics of the oligonucleotide-lipid nanoparticles that were loaded with fluorescent ODNs (LNP-ODN) as compared to free ODNs (Free-ODN).

FIG. 4A: Graph showing the oligonucleotide distribution in tumor tissue for a control, free-ODN, and LPN-ODN following i.v. administration.

FIG. 4B: Graph showing the oligonucleotide distribution in tumor tissue for a control, free-ODN, and LPN-ODN following i.v. administration.

FIG. 5: Schematic illustration of LN synthesis by ethanol dilution and the post insertion of Tf-PEG-DSPE used in Example A.

FIG. 6: CryoTEM micrograph of Tf-LNs entrapping G3139.

FIGS. 7A-7B: Colloidal stability of oligonucleotide formulations.

Lip, LN, Tf-Lip, Tf-LN or protamine-ODN complexes were stored in HBS buffer at 4° C. and particle sizes were measured by dynamic light scattering. The values in the plot represent the means of 3 separate experiments. Error bars were standard deviations, n=3. Lip, liposomes entrapping G3139; Tf-Lip, Tf-conjugated liposomes entrapping G3139.

FIG. 7A: Colloidal stability profiles of liposomes and LNs.

FIG. 7 B: Comparison of colloidal stability profiles of liposomes, LNs, and proticles (protamine-G3139 complexes).

FIG. 8: Serum stability of G3139 in Tf-LNs. Tf-LNs containing G3139 were mixed with serum at 1:4 volume ratio and incubated at 37° C. for different times and were analyzed by urea-PAGE. The density of G3139 bands in urea-PAGE was analyzed by ImageJ. Error bars stand for standard deviations, n=3.

FIG. 9A-9E: Uptake of Tf-LN G3139 in MV4-11 acute myeloid leukemia cells.

FIG. 9 A: Cells were treated with Tf-LN-G3139 spiked with 10% FITC-G3139 (green) at 37° C. for 15, 60 and 240 minutes, respectively, stained by DAPI (blue) and visualized on a confocal microscope.

FIG. 9 B: Cells were treated with Tf-LN-G3139 spiked with 10% FITC-G3139 for 4 hours at 37° C. and visualized on a fluorescence microscope.

FIG. 9C: Cells were treated with Tf-LN-G3139 spiked with 10% FITC-G3139 for 4 hours at 37° C. and cellular fluorescence was measured on a FACSCalibur flow cytometry. The X-axis indicates the cellular fluorescence intensity and the Y-axis indicates the cell count.

FIG. 9D: Cells, with or without 10×Tf in the culture medium, were treated with Tf-LN-G3139 spiked with 10% FITC-G3139 for 4 hours at 37° C. and cellular fluorescence was measured on a FACSCalibur flow cytometer. The X-axis indicates the cellular fluorescence intensity and the Y-axis indicates the cell count.

FIG. 9 E: Cells, with or without pre-incubated with 20 μM deferoxamine, were treated with Tf-LN-G3139 spiked with 10% FITC-G3139 for 4 hours at 37° C. and the fluorescence was measured on a FACSCalibur flow cytometry. Representative results are shown in this histogram with X-axis indicating the cellular fluorescence intensity and the Y-axis indicating the cell count.

FIGS. 10A-10D: TfR up-regulation by deferoxamine and its effect on Bcl-2 down-regulation by Tf-LN G3139 in leukemia cell lines.

FIG. 10A: Effect of deferoxamine-treatment on the TfR expression in leukemia cells. Cells were pretreated by 20 μM deferoxamine for 18 hours and then with 200 μg/ml FITC-Tf. Cellular fluorescence was measured by flow cytometry. Error bars stand for standard deviations, n=3.

FIG. 10B: Bcl-2 mRNA down-regulation in different cell lines treated by G3139 in various formulations. Cells were treated with PBS, 1 μM free G3139, G3139 in LN, or G3139 in Tf-LN. The treatment by Tf-LN G3139 was repeated on cells that were pre-treated with 20 μM deferoxamine for 18 hours. Bcl-2 mRNA levels were quantified by real-time RT-PCR after 48 hours. Error bars stand for standard deviations, n=3.

FIG. 10C: Bcl-2 protein down-regulation in leukemia cell lines treated by G3139 in various formulations. Cells were treated with PBS (1), 1 μM free G3139 (2), G3139 in LN (3), or G3139 in Tf-LN (4). In addition, treatment by Tf-LN G3139 was repeated on cells that were pre-treated with 20 μM deferoxamine for 18 hours (5). Bcl-2 protein levels were analyzed at 48 hours by Western blot. Upper panel represents the results of Western blot and lower represents its corresponding densitometry data. Error bars stand for standard deviations. Error bars stand for standard deviations, n=3.

FIG. 10D: Bcl-2 protein down-regulation by Tf-LN G3139 in K562 cells in the presence of 20 μM free holo-Tf in the culture medium. Bcl-2 protein levels were analyzed by Western blot at 48 hours after transfection. Upper panel represents the results of Western blot of Bcl-2 protein expression and lower represents its corresponding densitometry data. Error bars refer to standard deviations, n=3.

FIG. 11: Apoptosis measured by caspase-9 activities in K562 cells. Cells were incubated with PBS (1), 1 μM free G3139 (2), G3139 in LN (3), or G3139 in Tf-LN (4). In addition, the study was repeated on cells that were pre-treated with 20 μM deferoxamine for 18 hours (5). Cell apoptosis was evaluated via caspase-9 activities, as described in Materials and Methods (n=2).

FIGS. 12A-12D: Synthesis and pharmacokinetic properties of LNPs.

FIG. 12A: Flowchart of ODN-LNP preparation by EtOH dilution/diafiltration method.

FIG. 12B: Particle size distribution of ODN-LNPs after each step in a typical EtOH dilution/diafiltration process.

FIG. 12C: Plasma concentration-time profile of G4243-LNPs and free G4243 (G4243 is a fluorescein-labeled G3139) following tail vein i.v. bolus administration of 5 mg/kg of G4243-LNPs or free G4243 in DBA/2 mice (n=3).

FIG. 12D: Tumor accumulation profile of G4243-LNPs and free G4243 following tail vein i.v. bolus administration of 5 mg/kg of G4243-LNPs or free G4243 in DBA/2 mice (n=3). Each point represents Mean±SD of three mice.

FIGS. 13A-13B: Western blot analysis of Bcl-2 protein expression.

Human KB cells (FIG. 13A) and murine L1210 cells (FIG. 13B) were incubated with or without 1 μM G3139 for 72 hr, and the cells were harvested for Western-blot analysis. Ratios of Bcl-2 to β-actin were obtained by densitometry. There was a 2-nucleotide difference between the sequences of human and murine Bcl-2 mRNA.

FIGS. 14A-14B: Therapeutic efficacy of G3139-LNPs.

FIG. 14A: DBA/2 mice were inoculated s.c. with syngeneic L1210 cells 7 days prior to treatment. The mice received i.v. injections of PBS (pH 7.4), empty LNP, G3139, G3139-LNPs, or non-CpG containing G4126-LNPs on every 4th day until the mouse had tumor size of >1500 mm3. Low dose was 1.5 mg/kg of ODN, and high dose was 5 mg/kg of ODN. There were 5 mice in each group.

FIG. 14B: Comparison of antitumor effects of G3139, empty LNP, low dose G3139-LNPs (1.5 mg/kg), and high dose G3139-LNPs (5 mg/kg). Graphs show the mean tumor size (mm3), error bars indicated standard error (SE).

FIGS. 15A-15B: G3139-LNPs activated serum cytokine expression in mice. For serum cytokine detection, eight-week-old DBA/2 mice were injected i.v. with 1.5 mg/kg of G3139, G3139-LNPs, empty LNPs, or non-CpG containing G4126-LNPs. (FIG. 15A) IL-6 was measured at 4 hr, and (FIG. 15B) INF-γ was measure at 8 hr by ELISA. Three mice were used in each group.

FIGS. 16A-16C: G3139-LNPs enhanced intracellular cytokine expression in spleen cells and enlarged the spleen size.

FIG. 16A: For intracellular cytokines expression in spleen cells, eight-week-old DBA/2 mice were injected i.v. with 1.5 mg/kg of G3139, G3139-LNPs, and empty LNPs. There were 3 mice in each group. Spleen cells were harvested from mice 2 days after treatment, stained with fluorescence labeled MAbs, and measured by FACS.

FIG. 16B: Spleens harvested 7 days after i.v. administration of (a) G3139-LNPs (1.5 mg/kg of G3139), (b) free G3139 (1.5 mg/kg), and (c) empty LNPs in DBA/2 mice. Three mice were in each group.

FIG. 16C: Total cell numbers of the above spleen, G3139-LNP treated group has significantly more spleen cells than free G3139 (p=0.0017) and empty LNP treated groups (p<0.0001). (* indicates p<0.05, by Student's t test).

FIGS. 17A-17D: G3139-LNPs activated proliferation of innate immune cells. DBA/2 mice were treated with G3139-LNP, free G3139 or empty LNPs, and then injected i.p. with BrdU. Three mice were in each group. Twenty four hours after treatment, spleen cells were harvested, and the activation status of DX5+NK cells (FIG. 17A), CD11c+DCs (FIG. 17B), CD4+T cells (FIG. 17C), and CD8+T cells (FIG. 17D) were evaluated by BrdU incorporation rate. Results represent the average±SD of three independent experiments. (* indicates p<0.05, by Student's t test).

FIGS. 18A-18: G3139-LNPs induced IFN-γ production and activated innate and acquired immunity. INF-γ expression was determined in CD4 (FIG. 18A) and CD8 (FIG. 18B) cells 2 days or 7 days after treatment. Three mice were used in each group. Spleen cells were isolated and stained with INF-γ, CD4, and CD8-specific mAbs as described in Materials and Methods. Data showed the percentage of expressing cells identified by FACS. Results represent the average±SD of three independent experiments. (* indicates p<0.05, by Student's t test).

FIGS. 19A-19D: Immunohistochemistry (IHC) Staining of L1210 tumors. Frozen sections were prepared from tumors 7 days after treatment with G3139-LNPs (FIG. 19A), G3139 alone (FIG. 19B) or empty LNPs (FIG. 19C), and stained with anti-CD4, or anti-CD8 antibodies, or with hematoxylin & eosin (H&E). FIG. 19D, Tumor frozen sections from FIG. 19A, FIG. 19B and FIG. 19C groups were stained with anti-CD122.

FIGS. 20A-20G: In vitro assessment of free G3139 in Raji cell (FIG. 20A, FIG. 20B, FIG. 20C) and primary B-CLL cells (FIG. 20D, FIG. 20E, FIG. 20F, FIG. 20G) after 48 hr treatment.

FIG. 20A: Western blot analysis of bcl-2 expression in Raji cells. Raji cells were incubated with G3139 or G3622 (reverse sequence) at 1 uM, 2 uM and 5 uM for 48 hr. Subsequently, cells were lysed and analyzed by western blot study. The untreated cells (RPMI medium) were used for control.

FIG. 20B: Percentage of live Raji cells after 48 hr. The percentage of viable cells was determined for each sample by Annexin V/PI staining and was analyzed by flow cytometry. Data are representative of three experiments.

FIG. 20C: Changes in expression of surface markers in Raji cell after treatment with free G3139. Raji cells were incubated in the presence of G3139 at 1 uM. After 48 hr, expressions of CD40, CD80, CD86 and HLA-DR were measured by flow cytometry.

FIG. 20D: Two representative western blot results out of n=10 CLL patient cells. Primary B-CLL cells were incubated with G3139 at 1 uM, 2 uM and 5 uM for 48 hr and thereafter were collected and lysed for western blot analysis.

FIG. 20E: Quantification analysis of bcl-2 protein level by western blot (n=10). Average band intensities were determined by densitometry and data were presented as relative percentage compared to untreated cells control.

FIG. 20F: Relative B-CLL cell viability normalized to medium control. The percentage of viable cells was determined by Annexin V/PI staining and was analyzed by flow cytometry. Results present as means of n=12 independent experiments.

FIG. 20G: Fold changes of surface markers relative to medium control cell in B-CLL cells after G3139 treatment. Primary B-CLL cells were incubated in the presence of G3139 at 1 uM, 2 uM and 5 uM. After 48 hr, expressions of CD40, CD80, CD86 and HLA-DR were measured by flow cytometry. Results are shown as means of n=12 independent experiments.

FIG. 21: Assessment of rituximab against CD20 in B cell lines and primary B-CLL cells. Rituximab was fluorescently labeled with Alexa fluor 488 (green) using the method as mentioned in the part of materials and methods. 6 major B cell lines and the B cells isolated from patient with CLL were immunostained by Rituximab-Alexa 488 on ice for 30 mins, followed by washing twice and analyzing by flow cytometry. Data for cell lines are representative of three independent experiments and data for primary B-CLL cells are shown means of n=10 independent CLL patients.

FIGS. 22A-22B: AFM images of ODN loaded cationic liposomes (LPs). FIG. 22A—ODN encapsulated LP; FIG. 22B-ODN encapsulated Anti-CD20 LP. The solutions of ODN-LPs and ODN-anti-CD20 ILPs were dried on mica substrate. All measurements were recorded in both height and amplitude modes. Height images were presented here.

FIG. 23A-23: Effect of ODN loaded anti-CD20 cationic liposomes (anti-CD20 ILPs) on Raji malignant cells.

FIG. 23A: Comparison of rituximab directed CD20 receptor expression on Raji and Jurkat malignant cells. Herceptin was used as negative antibody control. Bindings of Rituximab-Alexa 488 and Herceptin-Alexa 488 to cells were determined by FACS. Cells were first incubated with Rituximab-Alexa 488 and Herceptin-Alexa 488 at 4 for 30 mins and thereafter were washed twice for flow cytometry analysis.

FIG. 23B: Binding study of free FAM-ODN and various LP formulated FAM-ODN on Raji (CD20+) and Jurkat (CD20−) cells. Cells were incubated with free ODN, naked LP, Her ILP and anti-CD20 ILP with the concentration of 1 uM at 37° C. for 1.0 hr and washed twice with cold PBS. The cells were analyzed by flow cytometry to detect the FAM-ODN fluorescence. Untreated cells were used as a negative control.

FIG. 23C: Blocking study of anti-CD20 ILP onto Raji cells by extra CD20 antibody (Rituximab) and CD52 antibody (Alemtuzumab). Raji cells were incubated with 1, 10, 100, or 1000 ug/ml CD20 or CD52 antibodies at 4 C for 30 mins before incubation of anti-CD20 ILP carrying FAM-ODN (1 uM) at 37 C for 1.0 hr. Untreated cells (bold line), cells treated with anti-CD20 ILP (thin solid line), cells blocked with Rituximab or Alemtuzuma (broken line) were assessed by flow cytometry.

Need FIG. 23D: Specificity study of anti-CD20 ILP on the mixed population of Raji and Jurkat cells. For surface staining, the mixed cells were kept with or without antibody on ice for 30 mins and washed tice with cold PBS. For the estimation of selective binding, the cells were incubated with anti-CD20 ILP (ODN, 0.5 uM) at 37° C. for 1.0 hr first. After being rinsed with cold PBS, the treated cells were further stained with APC labeled anti-CD 19-(the marker of B-Cell) or APC labeled anti-CD3 (the marker of T-Cell).

FIG. 23E: Western blot analysis of bcl-2 protein following exposure to free G3139 or various formulated G3139 in Raji cells. Raji cells were treated with free 2 uM G3139 or G3622 (reverse sequence) or 2 uM formulated ODNs in LPs for 48 hrs. Panel (A) represents the western blot expressions of Bcl2 protein and β-actin loading control and (B) represents its corresponding densitometry data.

FIG. 23F: Relative percentage of B-CLL cell viability normalized to medium control. The percentage of viable cells was determined by Annexin V/PI staining and was analyzed by flow cytometry. Results present as means of n=3 independent experiments.

FIG. 23G: Confocal microscopy analysis of uptake of fluorescently labeled ODN in Raji cells in vitro. Confocal microscopy was used to compare the uptake and cellular localization of free, LP, Her ILP and Anti-CDILP encapsulated 6-FAM labeled ODN (1 uM) 24 hr after transfection into Raji cells. After washing and fixation, the nucleus and membranes of cells were stained by DRAQ5. All images are at the identical magnification. DIC, differential interference contrast microscopy.

FIG. 24: Effect of ODN loaded anti-CD20 cationic liposomes (anti-CD20 ILPs) on primary B-CLL cells.

FIG. 24A: Binding study of free FAM-ODN and various LP formulated FAM-ODN on representative B-CLL cells. CD20 expression was shown on the top and the ability of anti-CD20 ILP mediated ODN delivery was assessed by flow histograms compared to free FAM-ODN and Her ILP mediated ODN delivery.

FIG. 24B: Dependence of anti-CD20 ILP mediated delivery on CD20 expressions of CLL patient cells. Two typical examples were selected to determine the correlation between targeting capacity of anti-CD20 ILP and CD20 expressions. The higher CD20 expression gives high intensity (left side), the lower CD20 expression shows almost no enhanced binding, comparable with the intensity of Her-ILP (right side). Cells were incubated with free FAM-ODN, FAM-ODN in Her ILP or anti-CD20 ILP with the concentration of 1 uM at 37° C. for 1.0 hr and washed twice with cold PBS. The cells were analyzed by flow cytometry to detect the FAM-ODN fluorescence. Untreated cells were used as a negative control. Specificity study of anti-CD20 ILP formulated FAM-ODN (FIG. 24C) and free FAM-ODN (FIG. 24D) in PBMC cells isolated CLL patient. For surface staining, the PBMC cells were kept with or without antibody on ice for 30 mins and washed twice with cold PBS. For the estimation of selective binding, the cells were incubated with free ODN (0.5 uM) or anti-CD20 ILP (ODN, 0.5 uM) at 37 C for 1.0 hr first. After being rinsed with cold PBS, the treated cells were further stained with APC labeled anti-CD19-(the marker of B-Cell) or APC labeled anti-CD3 (the marker of T-Cell).

FIG. 24E: Western blot analysis of bcl-2 protein following exposure to Her ILP or anti-CD20 ILP formulated G3139 and G3622 at 2 uM for 48 hr in B-CLL cells. The top panel represents the western blot expressions of Bcl2 protein and β-actin loading control and the below panel represents its corresponding densitometry data.

Need FIG. 24F: Relative percentage of B-CLL cell viability. B-CLL cells were treated with various conditions (ODN, 1 uM) at 37° C. for 48 hr. T hereafter, the percentage of viable cells was determined by Annexin V/PI staining and was analyzed by flow cytometry. The relative percentage of cell viability was obtained by normalizing to medium control. Results present as means of n=6 independent experiments.

FIGS. 25A-25B: CpG immunostimulation of G3139 can be significantly inhibited when encapsulated into anti-CD20 ILP.

FIG. 25A: Fold changes of surface markers relative to medium control in B-CLL cells after G3139 treatment. Primary B-CLL cells were incubated in the presence of free G3139, G3139-anti-CD20 ILP and G3139-anti-CD37 ILP. After 48 hr, expressions of CD40, CD80, CD86 and HLA-DR were measured by flow cytometry. Results are shown as means of n=6 independent experiments.

FIG. 25B: Fold changes of surface markers relative to medium control in B-CLL cells after ODN2006 treatment. Treatment conditions were similar with G3139. Results are shown as means of n=3 independent experiments.

FIGS. 26-27: CD37-ILN-Mcl-1 siRNA mediates down-regulation of Mcl-1 protein and promotes increased spontaneous apoptosis in CLL B cells.

FIG. 26: Specific delivery of CD37-ILN-FAM-ODN to B (CD19+) but not T (CD3+) cells in the peripheral blood mononuclear cells from CLL patients.

FIG. 27: Immunoblot analysis of protein extract from CLL B cells treated with CD37-ILN Mcl-1 siRNAs and control siRNAs shows decreased Mcl-1 protein in CD37-ILN-Mcl-1siRNA treated cells.

FIG. 28: Decreased viability as detected by Annexin V/PI staining in CLL B cells treated with CD37-ILN-Mcl-1 siRNAs compared to control siRNAs.

FIG. 29: Flow cytometry analysis of single and multi-antibody targeted liposomes. Enhanced FAM/ODN staining seen with dual targeted (CD20 and CD37-ILNs) compared to mono targeted ILNs.

FIG. 30: Schematic illustration showing Protein A based immunoliposomes dual or multi Ab targeted delivery system.

FIGS. 31A-31B: Graph showing a comparison of binding efficiency of Anti-CD ILPs prepared by two approaches: Post-insertion approach, and Protein A approach.

FIG. 32: Graph showing enhanced binding efficiency by dual-AB ILPs of Raji cells.

FIG. 33: Schematic illustration for the preparation of LPs and Tf-LPs by ethanol dilution and post insertion methods.

FIGS. 34A-34E: Cryo-TEM micrographs of polyplexes and LP nanoparticles.

FIG. 34A: Large amorphous complexes (arrowheads) of protamine/ODN, their internal structure is not visible.

FIG. 34B: “Thinner” and smaller amorphous complexes. White arrows show weaker contrast complexes that might be a dispersion of the protamine/ODN disordered complexes.

FIG. 34C: White arrow shows the onion-like structure of LPs.

FIG. 34D: Large variety of coexisting structures. The arrowhead shows a membrane “sac” that contains liposomes and the LP with the onion-like structure, and the white arrow is pointing at liposomes which are fused to an amorphous protamine/ODN complex.

FIG. 34E: Details of nanoparticles. White arrow shows lipids structure of liposome with amorphous core; white arrowhead shows the onion-like, multivescicular structure that contains a protamine/ODN amorphous layer that attaches the second and the third membrane layers.

FIG. 35A: Flow cytometry study of TfR expression: 1. cells stained with PE-isotype; 2. cells stained with PE-anti-TfR; 3. cells stained with PE-anti-TfR after DFO pre-treatment at 30 μM concentration for 18 hr.

FIG. 35B: The time-dependent uptake of FAM-GTI-2040-Tf-LPs by AML cells. Kasumi-1 cells were treated with 1 μM FAM-GTI-2040-Tf-LPs at 37° C. for various incubation time, washed twice in PBS and analyzed by flow cytometry.

FIG. 35C: Confocal microscopy images was used to compare the uptake and subcellular distribution of FAM-GTI-2040 delivered by Tf-LPs (1 μM) after Ohr and 4 hr incubation respectively. DIC: differential interference contrast (bright field) images. Green fluorescence of FAM-GTI-2040 and blue fluorescence of DRAQ5 were acquired, and merged images were produced.

FIGS. 36A-36B: R2 downregulation in Kasumi-1 AML cells under various conditions after 48 hr. Every sample was compared with Mock. Each column reflects the average of at least three independent experiments. The standard deviation is elucidated with an error bar. * indicates these data are statistically different from each other.

FIG. 36A: Upper panel shows representative western blot image. Lower panel shows the average densitometry data.

FIG. 36B: Improved R2 downregulation with DFO pre-treatment at 30 μM for 18 hr before the GTI-2040-Tf-LPs treatment. Upper panel shows representative western blot image. Lower panel shows the average densitometry data.

FIGS. 37A-37B: R2 downregulation in AML patient primary cells after 48 hr. Every sample was compared with Mock.

FIG. 37A: Upper panel shows representative western blot image. Lower panel shows the densitometry data.

FIG. 37B: Improved R2 downregulation with DFO pre-treatment primary AML patient cells from patient 3 after 48 hr. (1) Mock, (2) 1 μM Tf-LPs (GTI-2040), (3) 3 μM LPs (GTI-2040), (4) 3 μM Tf-LPs (GTI-2040), (5) 3 μM free GTI-2040, (6) 3 μM Tf-LPs (Scrambled), (7) cells treated with DFO treatment as control, (8) 1 μM Tf-LPs (GTI-2040)+DFO pre-treatment, and (9) 3 μM Tf-LPs (GTI-2040)+DFO pre-treatment. In samples 7, 8 and 9, cells were pre-treated with 30 μM DFO for 18 hours before the GTI-2040-Tf-LPs treatment. Upper panel shows a representative Western blot image. Lower panel shows the averages from densitometry analysis.

FIG. 38: Chemosensitization of Kasumi-1 cells toward Ara-C mediated by GTI-2040-Tf-LPs. Cells were treated with GTI-2040-Tf-LPs, free GTI-2040 or Scrambled-Tf-LPs at 1 μM concentration for 4 hr and then challenged the cells with Ara-C at various concentrations (0.0001-10 μM) for 48 hr. [symbol] (diamond) Mock+Ara-C; [symbol] (square) GTI-2040-Tf-LP+Ara-C; [symbol] (triangle) free GTI-2040+Ara-C; and [symbol] (floret) Scrambled-Tf-LP+Ara-C. Each point reflects the average of at least three independent experiments. Error bars indicate standard deviations.

FIGS. 39A-39B: Cryo-TAM micrographs: FIG. 39A the liposomes is oligolamellar; FIG. 39B the liposomes are unilamellar.

FIG. 40: Relative expressions of R1 gene in KB cells in different culture conditions.

FIG. 41: Schematic illustration showing strategies for efficiently loading cholesterol modified ODN/siRNAs into liposomal nanoparticles.

FIG. 42: Mcl-1 down-regulation by LPN-Mcl-1 siRNA formulation with Calcium (#5), compared to the formulation without Calcium (#4) and the negative siRNA control (#4). Additionally, LPN formulated Mcl siRNAs work more efficiently than free Mcl-1 siRNA (#2). In FIG. 42, 1. Mock; 2. Free Mcl-1 siRNA; 3. LP (no Ca2+, Mcl-1); 4. LP (no Ca2+, Negative); 5. LP (Ca2+, Mcl-1).

FIGS. 43A-43B: Graphs showing the changes of particles size after introducing calcium (FIG. 43A) and surface charge (zeta potential) (FIG. 43B) where the formulation is EggPC/Chol/PEG-DSPE—70/28/2, lipids/ODN 10/1; where #1 is Liposome alone; #2 is LP containing Chol-ODN; (no Ca2+); and #3 is LP containing Chol-ODN and Ca2+ (10 mM).

FIG. 43C: CryoTEM of Chol-ODN Encapsulated Liposomes without Ca2+ where the formulation is EggPC/Chol/PEG-DSPE—70/28/2, lipids/ODN 10/1.

FIG. 43D: CryoTEM of Chol-ODN Encapsulated Liposomes with Ca2+ where the formulation is EggPC/Chol/PEG-DSPE—70/28/2, lipids/ODN 10/1.

FIGS. 44A-44B: Graphs showing the changes of particles size after introducing calcium (FIG. 44A) and surface charge (zeta potential) (FIG. 44B) where the formulation is DC-chol/EggPC/PEG-DSPE—33.5/65/1/5, lipids/ODN 10/1; where #1 is Liposome, ODN; #2 is LP containing Chol-ODN; (no Ca2+); and #3 is LP containing Chol-ODN and Ca2+ (5 mM).

FIG. 44C: CryoTEM of Chol-ODN Encapsulated Liposomes without Ca2+ where the formulation is DC-chol/EggPC/PEG-DSPE—33.5/65/1/5, lipids/ODN 10/1.

FIG. 44D: CryoTEM of Chol-ODN Encapsulated Liposomes with Ca2+ where the formulation is DC-chol/EggPC/PEG-DSPE —33.5/65/1/5, lipids/ODN 10/1.

FIGS. 45A-45C: Mcl-1 down regulation in Raji cells by siRNA delivered via anti-CD20 conjugated nanoparticles (CD20 ILP) in CLL patient cells. #1. Mock; #2. LP (Mcl-1, 100 nM); #3. LP (negative, 100 nM); #4. CD37 ILP (Mcl-1, 100 nM); #5. CD37 ILP (negative, 100 nM); #6. CD20 ILP (Mcl-1, 100 nM); #7. CD20 ILP (negative, 100 nM).

FIG. 45A: Percentage of live Raji cells was determined by Annexin V/PI staining and was analyzed by flow cytometry.

FIG. 45B: Graph showing Mcl-1/Actin for #1-#7.

FIG. 45C: Western blot analysis of Mcl-1 protein and β-actin.

FIG. 46A: Western blot expressions of Bcl-2 protein and β-actin loading control.

FIG. 46B: RT-PCR analysis of Bcl-2 mRNA level. Results present as means of n=3 independent experiments. LNP Formulation: DC-Chol/EggPC/PEG-DSPE=30/68/2 (molar ratio) and lipids/ODN/protamine=12.5/1/0.3 (weight ratio).

FIG. 46C: CryoTEM image the structure of oligonucleotide-lipid nanoparticles. The coexistence of a two-layer lipid membrane (arrow) and a condensed multilamellar polyplexes is shown. The formulation of ODN-lipid nanoparticles is DC-Chol/EggPC/mPEG-DSPE=30/68/2 (molar ratio) and lipids/ODN/protamine=12.5/1/0.3 (weight ratio).

FIG. 47: Graph showing increased uptake of nanoparticle (LNP) formulated FAM-ODN (fluorescein-labeled ODN) by Raji Burkett's Lymphoma cells.

FIG. 48: Graph showing the therapeutic efficacy of antibody-targeted nanoparticles (ILPs).

FIGS. 49A-49B: BM preparation of ODN-LP (A) and (B) transferrin conjugated PEG-DSPE (Tf-PEG-DSPE):

FIG. 49A: Step 1: after mixing ODN with protamine/lipids and before dialysis, 2: after dual dialysis, 3: after 0.2 μl filtering, and 4: after post insertion with Tf-PEG-DSPE.

FIG. 49B: Holo-transferrin is reacted with Traut's reagent to from thiolated transferrin (HoloTf-SH) and reacted with maleimide-DSPE-PEG to form Tf-PEG-DSPE micelles for post insertion.

FIGS. 50A-50C: A 5-inlet MF device.

FIG. 50A: Schematic of the 5-inlet MF system.

FIG. 50B: Optical micrograph of the flow pattern at the two junctions (I and II) of the MF system.

FIG. 50C: Fluorescence micrograph of flow pattern at junction II. The volumetric flow rates used for rhodamine, fluorescein, and rhodamine were 200, 20, and 200 μL/min, respectively. Red and green color is rhodamine and fluorescein, respectively. Scale bar=250 μm.

FIG. 50D: Schematic illustration of optical MF system.

FIG. 51: Particle size distribution of ODN-LP produced by BM and MF methods following each step in an ethanol dialysis process. Step 1: after mixing ODN with protamine/lipids and before dialysis, 2: after dual dialysis, 3: after 0.2 μm filtering, and 4: after post insertion with Tf-PEG-DSPE. The average particle size for BM and MF lipopolyplex before and after post insertion of Tf-PEG-DSPE were 131.0±21.0 nm and 126.7±18.5 and 106.8±5.5 nm and 107.1±8.0 nm, respectively. The zeta potential of the LP nanoparticles before and after post insertion were +11.6±3.6 mV and +7.9±1.3 mV and +3.6±2.9 mV and +2.5±4.2 mV, respectively. Data are presented as mean±SD (n=4). p<0.05 indicated by * symbol.

FIGS. 52A-52B: Cryo-TEM images of LP nanoparticles prepared by (A) BM and (B) MF methods.

FIG. 52A: White arrowhead shows small multilamellar liposomes (i.e. onion ring like structure), white pentagon shows larger multilamellar liposomes, and white arrow shows large unilamellar vesicles.

FIG. 52B: White arrowhead shows small multilamellar liposomes (i.e. onion ring like structure), white pentagon shows larger multilamellar liposomes, white arrow shows large unilamellar vesicles, and black arrow shows bilamellar vesicles. Scale bar=100 nm.

FIG. 53: Determination of ODN encapsulation efficiency in LP nanoparticles by agarose gel electrophoresis. Lanes 1. ODN; 2. BM LP without 1% SDS; 3. MF LP without 1% SDS; 4. BM LP with 1% SDS; 5. MF LP with 1% SDS.

FIGS. 54A-54B: Effect of Bcl-2 downregulation by G3139. K562 cells were treated with free G3139, Tf conjugated G3139-containing liposomes produced by BM (BM Tf-LP), non-targeted G3139-containing liposomes produced by MF (MF LP), and Tf conjugated G3139-containing liposomes produced by MF (MF Tf-LP). G3139 concentration was 1 μM in all groups except for the untreated group. Bcl-2 protein and mRNA level were determined by Western blot and real-time RT-PCR, respectively. A representative Western blot of Bcl-2 protein expression (not shown), its corresponding densitometry data (FIG. 54A), and results of real-time RT-PCR analysis (FIG. 54B) at 24 and 48 h following treatment with different G3139-containing formulations are shown. p<0.05 and p<0.01 indicated by * and ** symbols, respectively. (n=3).

FIG. 55: Effect of G3139 concentration on Bcl-2 downregulation. A representative Western blot of Bcl-2 protein expression (not shown) and its corresponding densitometry data (FIG. 55) at 24 and 48 hr following treatment with free G3139 and G3139-containing formulations are shown (n=3). K562 cells were treated with BM Tf-LP and MF Tf-LP at G3139 concentration of either 0.5 μM or 1.0 μM. For free G3139, 1.0 μM was used.

FIGS. 56A-56B: Uptake of BM and MF lipopolyplexes containing FITC-labeled G3139 in K562 cells. Cells were treated with non targeted and targeted BM and MF LPs containing FITC-labeled G3139 as analyzed by (FIG. 56A) flow cytometry and (FIG. 56B) fluorescence microscopy at 400× magnification. 1 is untreated cell control, 2 is cells treated with non-targeted BM LP, 3 is cells treated with targeted BM Tf-LP, 4 and 6 are cells treated with non-targeted MF LP, and 5 and 7 are cells treated with targeted MF Tf-LP. Samples 2 to 5 were treated for 6 hr whereas 6 and 7 were treated for 24 hr. The ODN concentration used was 0.5 μM at a cell density 3×10⁵.

FIG. 57: A FCM bivariate plot of PI versus AV-FITC. The lower left (LL), lower right (LR), upper right (UR), and upper left (UL) quadrants correspond to cells that are negative for both dyes and are viable, positive only for AV-FITC which are cells in early stages of apoptosis and are viable, positive for both AV-FITC and PI which are cells in late stages of apoptosis or already dead, and positive for PI which are dead cells lacking membrane-based PS, respectively.

### DETAILED DESCRIPTION OF PREFERRED EMBODIMENT(S)

In a first broad aspect, there is provided herein an oligonucleotide-lipid nanoparticle comprising at least one oligonucleotide, at least one lipid and at least one complexation agent for the oligonucleotide formed by: i) mixing at least one lipid and at least one complexing agent and one or more cationic polymers, in a water miscible organic solvent to form a first mixture; ii) dissolving one or mixing two or more oligonucleotides in an aqueous buffer to form a second mixture; and, iii) injecting the first mixture into the second mixture, or mixing the first mixture and the second mixture under pressure, to form a third mixture; and iv) removing the organic solvent from the third mixture.

In another broad aspect, there is provided herein an oligonucleotide-lipid nanoparticle comprising at least one oligonucleotide, at least one lipid and at least one complexation agent for the oligonucleotide formed by: i) mixing at least one complexing agent and at least one oligonucleotide in an aqueous buffer to form a first mixture; ii) dissolving at least one lipid in a water-miscible solvent to form a second mixture comprised of liposomes or liposome precursors; iii) mixing the second mixture with the first mixture under pressure to from a third mixture; and iv) removing solvent from the third mixture.

In certain embodiments, the complexing agent comprises a divalent cation. In certain embodiments, the complexing agent comprises one or more of: Ca²⁺, Mg²⁺, pentaethylenehexamine (PEHA), spermine, protamine, polylysine, chitosan, and polyethyleneimine (PEI).

In certain embodiments, the water miscible organic solvent comprises one or more of ethanol, isopropanol, and tert-butanol containing 0 to about 50% water.

In certain embodiments, the third mixture has a final organic solvent-to-water ratio ranging from about 30/70 to about 50/50.

In certain embodiments, the oligonucleotide-lipid nanoparticle further includes at least one targeting ligand.

In certain embodiments, the oligonucleotide-lipid nanoparticle further include at least one additional functional component.

In certain embodiments, the oligonucleotides include one or more of: antisense deoxyribonucleotides, small interfering RNAs (siRNAs), microRNAs (miRNAs), CpG ODNs, or antisense deoxyribonucleotides, including combinations of oligonucleotides of the same and of different classes. In certain embodiments, the oligonucleotides contain one or more chemical modifications configured to increase the stability and/or lipophilicity of the oligonucleotides. In certain embodiments, the chemical modifications comprises one or more of a phosphorothioate linkages between the nucleotides, a cholesterol or lipid conjugated to the oligonucleotide at the 5′ or 3′ end, and 2′O-methylation on the ribose moieties.

In certain embodiments, the lipid comprises one or more of: a) cationic or anionic lipids or surfactants; b) neutral lipids or surfactants; c) cholesterol; and d) PEGylated lipids or surfactants. In certain embodiments, the lipids are configured to promote electrostatic interaction directly or indirectly with anionic oligonucleotides.

In certain embodiments, the cationic lipid includes a titratable headgroup with pKa between 5 and 8. In certain embodiments, the cationic lipid comprises one or more of: 3 alpha-[N-(N′,N′-dimethylaminoethane)-carbamoyl] cholesterol hydrochloride (DC-Chol), or 1,2-dioleoyl-3-(dimethylamino)propane (DODAP). In certain embodiments, the cationic lipid is configured with a permanent cationic charge at physiological pH with pKa above 8. In certain embodiments, the cationic lipid comprises one or more of: 1,2-dioleoyl-3-trimethylammonium-propane (DOTAP) or dioctadecyldimethyl ammonium bromide (DDAB).

In certain embodiments, the neutral lipids are configured to increase bilayer stability. In certain embodiments, the neutral lipids comprises a phosphatidylcholine. In certain embodiments, the neutral lipid is configured to regulate endosomolytic activity of the nanoparticle. In certain embodiments, the neutral lipid comprises dioleoylphosphatidylethanolamine (DOPE), alpha-tocopherol, triolein, or diolein.

In certain embodiments, the nanoparticle includes cholesterol to enhance the bilayer stability.

In certain embodiments, the PEGylated lipid is configured to promote colloidal stability and/or to prolong in vivo circulation time. In certain embodiments, the PEGylated lipid comprises one or more of: methoxy-polyethyleneglycol-distearoylphosphatidyl-ethanolamine (mPEG-DSPE), TPGS, Tween-80 and other polysorbates, Brij series surfactants, and poly(oxyethylene) cholesteryl ethers (PEG-chol).

In certain embodiments, the nanoparticle further includes one or more anionic lipids. In certain embodiments, the anionic lipid comprises one or more of: cholesteryl hemisuccinate (CHEMS), dicetylphosphate, phosphatidylglycerol, alpha-tocopherol succinate, and oleic acid.

In certain embodiments, the targeting ligand is conjugated to a hydrophobic anchor with or without a linker. In certain embodiments, the hydrophobic anchor comprises one or more of: a lipid or a lipid-like molecule, an alpha-tocopherol derivative, or a cholesterol derivative.

In certain embodiments, the targeting ligand comprises one or more of: transferrin, folate, oligosaccharides, and tissue or cell-specific antibodies, and is conjugated to a hydrophobic anchor comprising one or more of: phosphatidylethanolamine derivative, a lipophilic molecule, and cholesterol.

In certain embodiments, the oligonucleotide-lipid nanoparticle includes one or more additional functional components, including fusogenic peptides, membrane lytic polymers, and nuclear localization signal peptides.

In another broad aspect, there is provided herein a method for protecting an oligonucleotide from degradation by nucleases and prolonging systemic circulation time in vivo, the method comprising loading an oligonucleotide into a lipid nanoparticle, whereby the oligonucleotide-lipid nanoparticle is formed.

In certain embodiments, the in vivo circulation time is further extended by grafting one or more PEG polymers onto a surface of the oligonucleotide-lipid nanoparticle.

In certain embodiments, the oligonucleotide-lipid nanoparticle is formed by: i) mixing at least one lipid and at least one complexing agent, including, but not limited to a divalent cation or one or more cationic polymers, in a water miscible organic solvent, with or without up to 50% water, to form a first mixture; ii) mixing one or more oligonucleotides in an aqueous buffer to form a second mixture; and, iii) injecting the first mixture into the second mixture or mixing the two under pressure to form a third mixture; and iv) removing solvent from the third mixture.

In certain embodiments, the oligonucleotide-lipid nanoparticle is formed by: i) mixing at least one complexing agent including, but not limited to a divalent cation or one or more cationic polymers, and at least one oligonucleotide in an aqueous buffer to form a first mixture; ii) dissolving at least one lipid in a water miscible solvent containing 0 to about 50% water to form a second mixture comprised of liposomes or a liposome precursor; iii) mixing the second mixture with the first mixture under pressure to from a third mixture; and iv) removing solvent from the third mixture.

In certain embodiments, the method includes an additional step of particle size reduction is added to make the nanoparticle size smaller and more uniform, and the removal step comprises diluting and/or dialyzing the third mixture. In certain embodiments, the additional step of particle size reduction is added by sonication to make the nanoparticle size smaller and more uniform, and the removal step comprises diluting and/or dialyzing the third mixture. In certain embodiments, the additional step of particle size reduction is added by high pressure homogenization to make the nanoparticle size smaller and more uniform, and the removal step comprises diluting and/or dialyzing the third mixture. In certain embodiments, the by high pressure homogenization comprises to make the particle size smaller and more uniform.

In certain embodiments, the removal step is accomplished by using tangential-flow diafiltration that leads to exchanging the nanoparticles into an aqueous buffer and adjusting the oligonucleotide-lipid nanoparticles to a desired concentration.

In certain embodiments, the method is configured for large-scale production for clinical applications.

In certain embodiments, the method further includes one or more steps: complexing or conjugating a targeting ligand to a lipid bilayer for “ligand conjugation”, or adding a lipid-conjugated targeting ligand followed by incubation for “post-insertion” of the ligand; sterilizing the lipid nanoparticles by filtration; and lyophilizing the oligonucleotide-lipid formulation in the presence of a lyoprotectant to achieve long term stability under mild storage conditions and easy reconstitution of the aqueous formulation at the point of use.

In certain embodiments, the filtration of the lipid nanoparticles is through a sterile filter of ˜0.2 μM. In certain embodiments, the lyoprotectant comprises a disaccharide. In certain embodiments, the lyoprotectant comprises about 5 to about 20% sucrose.

In another broad aspect, there is provided herein a method for delivering oligonucleotides to a solid tumor, the method comprising using long-circulating oligonucleotide/lipid-nanoparticles, wherein the oligonucleotide/lipid-nanoparticle exhibits an enhanced permeability and retention (EPR) effect, which results in increased accumulation in tumor tissues relative to normal tissues.

In another broad aspect, there is provided herein an oligonucleotide-lipid nanoparticle, formed by a microfluidic focusing process which produces nanoparticle having a substantially uniform size and structure, increased oligonucleotide loading efficiency and with better transfection efficiency and less cytotoxicity.

In another broad aspect, there is provided herein a microfluidic hydrodynamic focusing method for preparing lipopolyplex containing one or more antisense oligodeoxynucleotides configured for targeting one or more antiapoptotic proteins under- or over-expressed in a cancer-associated disorder.

In another broad aspect, there is provided herein a lipopolyplex composition comprising one or more oligonucleotides, one or more protamines, and one or more lipids, present in about oligonucleotide:protamine:lipids (1:0.3:12.5 wt/wt ratio).

In another broad aspect, there is provided herein a lipopolyplex composition comprising one or more oligonucleotides, one or more protamines, and one or more lipids, wherein the lipids include DC-Chol:egg PC:PEG-DSPE present in about 40:58:2 mol/mol %.

In another broad aspect, there is provided herein a microfluidic process for making nanoparticle comprising substantially controlling flow conditions and mixing process of reagents at a micrometer scale to synthesize nanoparticles having a substantially uniform and well-defined size, structure, and pharmacological functions.

In another broad aspect, there is provided herein nanoparticles useful for efficient delivery of single stranded or duplexed DNA or RNA oligonucleotide compounds to cancer cells.

In certain embodiments, the nanoparticles comprise one or more of: a first component configured for stabilizing one or more oligonucleotides in serum and for increasing delivery efficiency; a second component configured for shielding lipopolyplexes (LPs) from the serum proteins and for targeting cell surface receptors; and a third component configured for further stabilizing the LPs against plasma protein adsorption and clearance by the reticuloendothelial system of a subject, thereby achieving prolonged blood circulation time.

In another broad aspect, there is provided herein a stable lipopolyplex formulation that yields nanoparticles of medium diameters of less than about 250 nm, high ODN entrapment efficiency, colloidal stability, long circulation time, and specific targeting to cancerous cells.

In another broad aspect, there is provided herein a microfluidic device for making nanoparticles, comprising multiple channels, wherein the channel widths are varied.

In another broad aspect, there is provided herein a method for making a microfluidic device, comprising: laminating a PMMA film to form closed microchannels having inlets and outlets by passing a PMMA/film sandwich through a thermal laminator; sonicating the PMMA plates; drying the PMMA plates; and bonding fluidic connectors onto the inlets and outlet on the PMMA plate by applying a UV curing adhesive around a perimeter of each of the connectors, wherein the connectors are aligned over inlet/outlet openings; and curing the adhesive by exposure to UV irradiation.

In another broad aspect, there is provided herein a microfluidic device for making oligonucleotide-lipid nanoparticles, comprising at least three inlet ports and at least one outlet port, each inlet port being connected to a separate injection device; the device being configured such that: i) when a first fluid stream is introduced into each of the first and second inlet ports, the first fluid stream is split into two side microchannel streams at the third inlet port; and ii) when a second fluid stream is introduced in the third inlet port, a product stream is formed that is collected at the outlet port.

In another broad aspect, there is provided herein a microfluidic device for making oligonucleotide-lipid nanoparticles, comprising at least five inlet ports and at least one outlet port, each inlet port being connected to a separate injection device; the device being configured such that: i) when a first fluid stream is introduced into the first inlet port and a second fluid stream is introduced into the second inlet port, the first fluid stream is split into two side microchannel streams at the third inlet port; ii) when a third fluid stream is introduced in the third inlet port, a first product stream is formed at a first junction; iii) when a fourth fluid stream is introduced into the fourth inlet port and a fifth fluid stream is introduced into the fifth inlet port at a point downstream of the first junction, the fourth fluid stream and the fifth fluid stream contact the first product stream to form a second product stream at a second junction; the second product stream being collected at the outlet port.

In certain embodiments, the injection device comprises a syringe pump configured to deliver one or more of: protamine or lipids or protamine/lipids or ODN solution.

In another broad aspect, there is provided herein a method of oligonucleotide-lipid nanoparticles, comprising: i) introducing a first fluid stream into a first inlet port; ii) introducing a second fluid stream into a second inlet port and a third fluid stream into a third inlet port, the second and third inlet ports being positioned on opposing sides of the first inlet port, the second and third fluid streams hydrodynamically focusing the first fluid stream into a narrow stream to form a first product stream at a first junction; and iii) introducing downstream of the first junction a fourth fluid stream into a fourth inlet port and a fifth fluid stream into a fifth inlet port, the fourth and fifth inlet ports being positioned downstream to and on opposing sides of the first junction, the fourth and fifth fluid streams hydrodynamically focusing the first product stream into a narrow stream to form a second product stream.

In certain embodiments, the first fluid stream comprises an oligonucleotide (ODN) solution; the second fluid comprises a protamine sulfate solution stream; the third fluid comprises a protamine sulfate solution stream; the first product stream comprises ODN/protamine nanoparticles formed via electrostatic interaction between negatively charged ODN and positively charged protamine sulfate; the fourth fluid stream comprises a lipid stream; the fifth fluid stream comprises a lipid stream; and the second product stream comprises ODN/protamine/lipids nanoparticles or lipopolyplexes.

In certain embodiments, the second product stream comprises nanoparticles having a final weight ratio of ODN:protamine:lipids of about 1:0.3:12.5 and an ethanol concentration about 30 to about 70%. In certain embodiments, the flow rates for ODN, protamine, and lipids streams are about 20, about 20, and about 450 μL/min, respectively, and, optionally, are controlled independently. In certain embodiments, the ODN and protamine were prepared in sodium citrate buffer (20 mM, pH 4), and the lipids mixture was in 100% ethanol.

In certain embodiments, the first fluid stream comprises a protamine/lipids mixture stream; the second fluid comprises a first oligonucleotide (ODN) stream; the third fluid comprises a second oligonucleotide (ODN) stream; the first product stream comprises ODN/protamine/lipids stream; the fourth fluid stream comprises a protamine/lipids stream; the fifth fluid stream comprises a protamine/lipids stream; and the second product stream comprises ODN/protamine/lipids nanoparticles or lipopolyplexes.

In certain embodiments, the second product stream comprises nanoparticles having a final weight ratio of ODN:protamine:lipids of about 1:0.3:12.5 and an ethanol concentration about 30 to about 70%. In certain embodiments, the flow rates for protamine/lipids, ODN, and protamine/lipids streams are about 200, about 20, and about 200 μL/min, respectively, and, optionally, are controlled independently.

In certain embodiments, the method includes where protamine (delivered via the second and third inlet ports, and lipids, delivered via the fourth and fifth inlet ports, or protamine/lipids streams, delivered via the second, third, fourth and fifth inlet ports, are injected first and thereafter the ODN stream is injected via the first inlet port.

In certain embodiments, the method includes where after the ODN stream has entered and the hydrodynamic focusing established, the products are flowed for a further period of time to allow for achieving a steady state before being collected at the outlet port.

In certain embodiments, the method includes where the magnitude of the hydrodynamic focusing is controlled by altering the flow rate ratio (FR) of the second and third streams to the first stream, wherein FR is the ratio of total flow rate to the first stream flow rate.

In certain embodiments, the method includes where programmable syringe pumps are used to control the fluid flow rates independently.

In certain embodiments, the method further includes treating the second product stream by vortexing and sonicating, followed by dialyzing against a buffer to raise the pH to neutral in order to remove unbound ODN, reduce ethanol, and to partially neutralize cationic DC-Chol.

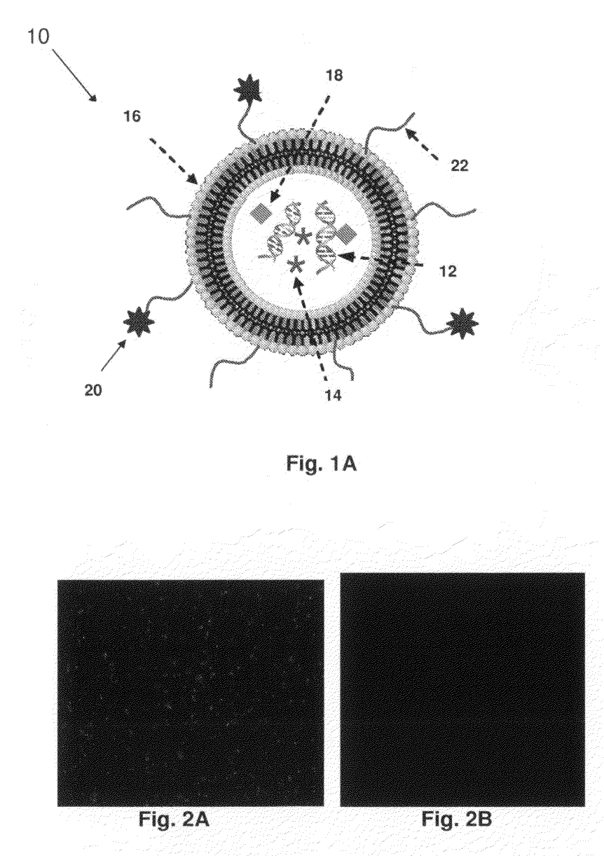

A schematic illustration of one embodiment of an oligonucleotide-lipid nanoparticle 10 is shown in FIG. 1. The oligonucleotide-lipid nanoparticle 10 includes an oligonucleotide 12, at least one complexing/condensing agent 14 at least partially encapsulated in a lipid nanoparticle 16. One or more functional additives 18 can also be at least partially encapsulated in the lipid nanoparticle 16. In the embodiment shown in FIG. 1, the oligonucleotide-lipid nanoparticle 10 includes one or more targeting ligands 20 that include a linker 22, such as PEG.

The combinations of different types of oligonucleotides (e.g., combinations of two of more siRNA and/or miRNA), including different classes of oligonucleotides (e.g., antisense ODN combined with siRNA) in the same oligonucleotide-lipid nanoparticle provides a very effective delivery mechanism, which, until now, has never before been proposed.

The delivery of oligonucleotide combinations via co-loading into the lipid nanoparticles is especially useful and provides a synergistic interplay of the oligonucleotides. Using the oligonucleotide-lipid nanoparticles, there can now be formulated siRNA combinations that are effective in gene silencing in vitro that can be delivered using a single delivery mechanism.

The oligonucleotide-lipid nanoparticles are also useful for gene silencing since cholesterol-modified oligonucleotides can be used for gene silencing when incorporated as a component of the oligonucleotide-lipid nanoparticles.

The modified oligonucleotides have a very high (−100%) incorporation into oligonucleotide-lipid nanoparticles and the resulting particles are very compact in size (<200 nm in diameter).

In another broad aspect, there is provided herein a method for the synthesis of lipid nanoparticle compositions. The solvent injection/self assembly method of oligonucleotide-lipid nanoparticles synthesis is tunable and scalable and is uniquely suitable for large-scale production. The mechanism of oligonucleotide-lipid nanoparticles formation is based on electrostatic complexation and recruitment of lipids as surfactants.

The method described herein provides a synthetic strategy that successfully produces oligonucleotide-lipid nanoparticles with a desired particles size distribution and colloidal stability in the presence of serum. The tangential flow diafiltration method of removing solvent from the oligonucleotide-lipid nanoparticles formulation allows the process to be adapted to large-scale production of oligonucleotide-lipid nanoparticles for commercialization. By varying injection fluid velocity (or fluidic pressure), the process and the particle size can bed adjusted.

In one particular embodiment, the method includes: 1) dissolving one or more oligonucleotides in an aqueous buffer to form a first solution; 2) codissolving at least one lipid and at least one cationic polymer in a water miscible organic solvent, such as ethanol and tert-butanol with 0-40% of water, for forming a second solution; 3) injecting the second solution into the first solution under relatively high pressure to obtain a final solvent-to-water ratio ranging from about 30/70 to about 50/50 to form a third solution; whereby the oligonucleotide-lipid nanoparticles are formed; and, 4) removing solvent from the third solution. In certain embodiments, the removal step can be accomplished by using a tangential-flow diafiltration, for exchanging into an aqueous buffer and for adjusting the oligonucleotide-lipid nanoparticles to a desired concentration. The solvent injection and diafiltration method can be readily scaled up. Another advantage is that the method for making such oligonucleotide-lipid nanoparticles has a high recovery yield and a high encapsulation efficiency of the oligonucleotides by the lipids.

After the formation of the oligonucleotide-lipid nanoparticles, the lipid nanoparticles can be sterilized by filtration, for example, through a 0.2 micron membrane. Also, the process can include lyophilizing the oligonucleotide-lipid formulation. In certain embodiments, lyoprotectant, typically a disaccharide solution, such as 10-20% sucrose, can be included in the vehicle solution.

The oligonucleotide-lipid nanoparticles are useful when used in complexing or conjugating a targeting ligand to a lipid bilayer for “ligand conjugation,” or adding a lipid-conjugated targeting ligand followed by incubation for “post-insertion” of the ligand.

The formation of the oligonucleotide-lipid nanoparticles in this process is believed by the inventors herein to be based on electrostatic complexation and interfacial free energy reduction. The particle size is, at least in part, dependent on the velocity of liquid stream during the injection of the second solution into the first solution, as well as on the concentrations of the first and second solutions. At the time of the injection, the cationic polymer and/or cationic lipid rapidly form electrostatic complexes with the oligonucleotides (which carry anionic charges). These electrostatic complexes have diameters in the nanometer ranges, and possess high interfacial free energy (γ). In this process, the recruitment of neutral and PEGylated lipids (which are surfactants that can adsorb to the interface and reduce the high interfacial free energy (γ)) thus forming substantially uniform and stable lipid-coated nanoparticles of oligonucleotides.

The oligonucleotide-lipid nanoparticles have a greatly desired small particle size and excellent colloidal stability. The oligonucleotide-lipid nanoparticles have a low toxicity, a desirably long circulation time in vivo, and have a high target cell uptake and transfection efficiency.

These advantages will now be illustrated by the following non-limiting examples. The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference.

The following examples are intended to illustrate certain preferred embodiments of the invention and should not be interpreted to limit the scope of the invention as defined in the claims, unless so specified.

### Example 1

Oligonucleotide-lipid nanoparticles were formed, as shown in Table 1 below.

TABLE 1

| | Formulation for Oligonucleotide- | Particle | Zeta |
|---|---|---|---|
| | lipid nanoparticles (LPN) | size | potential |
| 1 | DC-Chol:EggPC:PEG-DSPE = 30:65:5 | 27.5 nm | 4.7 ± 0.42 mV |
| | ODN:Lipids = 1:12.5 | mv | |
| 2 | DC-Chol:EggPC:PEG-DSPE = 25:73.5:1.5 | 44.95 nm | 11.3 ± 0.96 mV |
| | ODN:Lipids = 1:12.5 | mv | |
| 3 | DC-Chol:EggPC:PEG-DSPE = 30:65:5 | 42.4nm | 17.47 ± 0.57 mV |
| | ODN:Lipids:PEHA = 1:12.5:0.3 | mv | |
| 4 | DC-Chol:EggPC:PEG-DSPE = 30:65:5 | 28.9 nm | 16.04 ± 0.36 mV |
| | ODN:Lipids:protamine = 1:12.5:0.3 | mv | |

### Example 2

Oligonucleotide-lipid nanoparticles were formed, as shown in Table 2 below.

| | | Particle | Zeta | ODN loading |
|---|---|---|---|---|
| | Formulation | size | potential | efficiency |
| 1 | DC-Chol:EggPC:PEG-DSPE = 33.5:65:1.5 | 63.4 nm | 20.16 ± 0.43 mV | >95% |
| | ODN:Lipids:Spermidine= 1:15.0:0.4 | 3.38 ± 0.41 mv | | |
| 2 | DC-Chol:EggPC:PEG-DSPE = 33.5:65:1.5 | 50.65 nm | 23.77 ± 1.00 mV | >95% |
| | ODN:Lipids:PEHA = 1:15.0:0.4 | 5.00 ± 0.76 mv | | |
| 3 | DC-Chol:EggPC:PEG-DSPE = 33.5:65:1.5 | 56.03 nm | 20.27 ± 0.55 mV | 83.1% |
| | ODN:Lipids:PEI-Rhodamine(2K) = 1:15.0:0.4 | | | |
| 4 | DC-Chol:EggPC:PEG-DSPE = 33.5:65:1.5 | 60.1 nm | 17.06 ± 1.28 mV | 87.5% |
| | ODN:Lipids:PEI-RH(25K) = 1:15.0:0.4 | 8.96 ± 1.00 mv | | |
| 6 | DDAB:Chol:EggPC:PEG-DSPE = 25:25:46:4 | 262.1 nm | 18.73 ± 0.56 mV | >95% |
| | ODN:Lipids:PEHA = 1:15.0:0.4 | | | |

### Example 3

FIG. 2A and FIG. 2B show the differences in cellular uptake of transferrin-conjugated oligonucleotide-lipid nanoparticles and that of free oligonucleotides. FIG. 2A shows K562 human leukemia cells treated with transferrin oligonucleotide-lipid nanoparticles. In contrast, FIG. 2B shows K562 cells treated with free oligonucleotide. —The data showed that targeted nanoparticles were much more efficiently taken up by the cells than the free oligonucleotide.

### Example 4

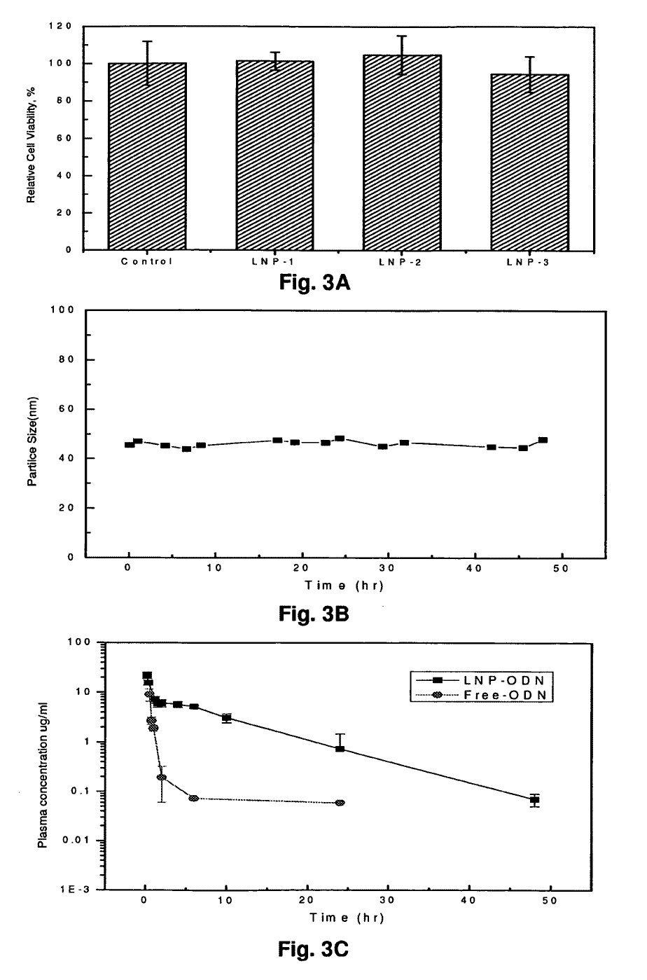

A study of the cytotoxicity of the oligonucleotide-lipid nanoparticles was conducted. FIG. 3A is a graph showing the relative cell viability for a control and for the oligonucleotide-lipid nanoparticle formulations as shown in Table 1 for LNP-1. LPN-2 and LPN-3. The data demonstrated that these nanoparticle formulations have minimal cytotoxicity.

### Example 5

A study of the colloidal stability of the oligonucleotide-lipid nanoparticles was conducted. FIG. 3B is a graph showing the particle size (nm) of the oligonucleotide-lipid nanoparticles over time. The data indicated excellent long-term colloidal stability of the nanoparticles.

### Example 6

A study of the pharmacokinetics of the oligonucleotide-lipid nanoparticles that were loaded with fluorescent ODNs was conducted. FIG. 3C shows the plasma clearance kinetics of the oligonucleotide-lipid nanoparticles that were loaded with fluorescent ODNs (LNP-ODN) as compared to free ODNs (Free-ODN) over time. The data showed prolonged circulation time for the nanoparticles relative to the free ODN.

### Example 7

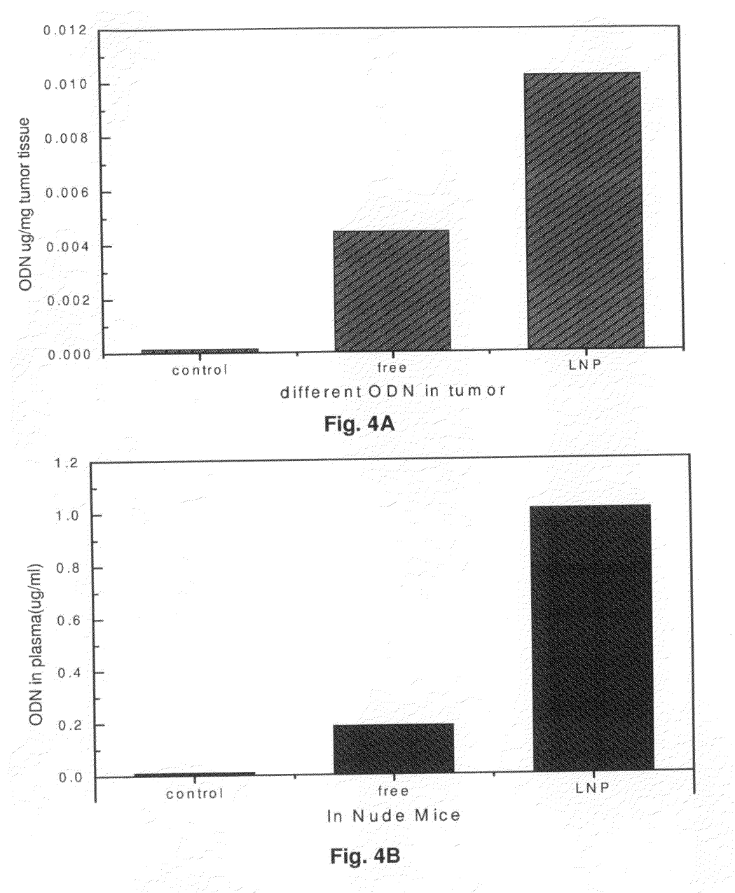

A study of the biodistribution of the oligonucleotides in the oligonucleotide-lipid nanoparticles in nude mice carrying K562 xenograft tumors was conducted. FIG. 4A is a graph that shows the oligonucleotide distribution in tumor tissue for a control, free-ODN, and LPN-ODN.

### Example 8

A study of the biodistribution of the oligonucleotides in the oligonucleotide-lipid nanoparticles in the plasma levels of nude mice carrying K562 xenograft tumors was conducted. FIG. 4B is a graph that shows the oligonucleotide distribution in tumor tissue for a control, free-ODN, and LPN-ODN.

While not wishing to be held merely to the following, the Examples of Uses herein provide evidence of the wide applicability of the present invention.

### Examples of Uses

### Example A

Antisense oligonucleotide G3139-mediated down-regulation of Bcl-2 is a potential strategy for overcoming chemoresistance in leukemia. However, the limited efficacy shown in recent clinical trials calls attention to the need for further development of novel and more efficient delivery systems. In order to address this issue, transferrin receptor (TfR)-targeted, protamine-containing lipid nanoparticles (Tf-LNs) were synthesized as delivery vehicles for G3139. The LNs were produced using an ethanol dilution method and a lipid-conjugated Tf ligand was then incorporated by a post-insertion method.

The resulting Tf-LNs had a mean particle diameter of −90 nm, G3139 loading efficiency of 90.4%, and showed a spherical structure with one to several lamellae when imaged by cryogenic transmission electron microscopy. Antisense delivery efficiency of Tf-LNs was evaluated in K562, MV4-11 and Raji leukemia cell lines. The results showed that Tf-LNs were more effective than non-targeted LNs and free G3139 (p<0.05) in decreasing Bcl-2 expression (by up to 62% at the mRNA level in K562 cells) and in inducing caspase-dependent apoptosis. In addition, Bcl-2 down-regulation and apoptosis induced by Tf-LN G3139 were shown to be blocked by excess free Tf and thus were TfR-dependent. Cell lines with higher TfR expression also showed greater Bcl-2 down-regulation. Furthermore, up-regulation of TfR expression in leukemia cells by iron chelator deferoxamine resulted in a further increase in antisense effect (up to 79% Bcl-2 reduction in K562 at the mRNA level) and in caspase-dependent apoptosis (by ˜3-fold) by Tf-LN. Tf-LN mediated delivery combined with TfR up-regulation by deferoxamine appears to be a potentially promising strategy for enhancing the delivery efficiency and therapeutic efficacy of antisense oligonucleotides.

### Introduction to Example A

Antisense oligonucleotides, typically of 15-20 nucleotides in length, are designed to target specific mRNA sequences through Watson-Crick hybridization, resulting in the destruction or disablement of the target mRNA. G3139 (oblimersen sodium, Genasense™) is an 18-mer phosphorothioate oligonucleotide targeting the anti-apoptotic protein Bcl-2. Since Bcl-2 is frequently overexpressed in tumor cells and is implicated in drug resistance, down-regulation of Bcl-2 using G3139 can potentially restore chemosensitivity in leukemia cells. Combinations of G3139 with chemotherapeutics have recently been studied for the treatment of acute myelogenous leukemia (AML) and chronic lymphocytic leukemia (CLL). However, clinical efficacy of G3139 has been shown to be limited, believed to be due to the rapid clearance of G3139 from blood circulation by metabolism and excretion, as well as the low permeability of the drug across the cellular membrane. Although the phosphorothioate backbone of G3139 renders it less sensitive to nucleases, other remaining obstacles in the G3139 delivery pathway still need to be overcome.

Example A, describes a oligonucleotide carrier, Tf-LNs, which incorporated Tf as targeting ligand and protamine as an oligonucleotide complexing agent. The Tf-LNs show excellent physiochemical properties and oligonucleotide delivery efficiency. The Tf-LNs, loaded with G3139, were evaluated for Bcl-2 downregulation and pro-apoptotic activities in leukemia cell lines. Tf-LNs were shown to have high efficiency and TfR specificity in delivery of G3139 and effectively reduced Bcl-2 expression and increased cell apoptosis among leukemia cells. Furthermore, the delivery efficiency via Tf-LNs was further enhanced by deferoxamine, which up-regulated TfR expression on leukemia cells.

### Materials and Methods for Example A

Reagents. 3β-[N-(N′,N′-dimethylaminoethane)-carbamoyl] cholesterol (DC-chol), egg phosphatidylcholine (egg PC) and distearoyl phosphatidylethanolamine-N-[maleimide-polyethylene glycol, MW 2000] (Mal-PEG₂₀₀₀-DSPE) were purchased from Avanti Polar Lipids (Alabaster, Ala.). Methoxy-PEG₂₀₀₀-DSPE (PEG₂₀₀₀-DSPE) was purchased from Genzyme Corporation (Cambridge, Mass.). Human holo-transferrin (Tf), 2-iminothiolane (Traut's reagent), protamine sulfate, and other chemicals were purchased from Sigma Chemical Co. (St. Louis, Mo.). All tissue culture media and supplies were purchased from Invitrogen (Carlsbad, Calif.).

Antisense oligonucleotides. All oligonucleotides used in this example were fully phosphorothioated. G3139 (5′-TCT CCC AGC GTG CGC CAT-3′) [SEQ ID NO: 1] and its fluorescence-labeled derivative, G4243 (FITC-G3139).

Cell culture. All leukemia cell lines were cultured in RPMI 1640 media supplemented with 10% heat-inactivated fetal bovine serum (FBS) (Invitrogen), 100 U/mL penicillin, 100 μg/mL streptomycin, and L-glutamine at 37° C. in a humidified atmosphere containing 5% CO₂.

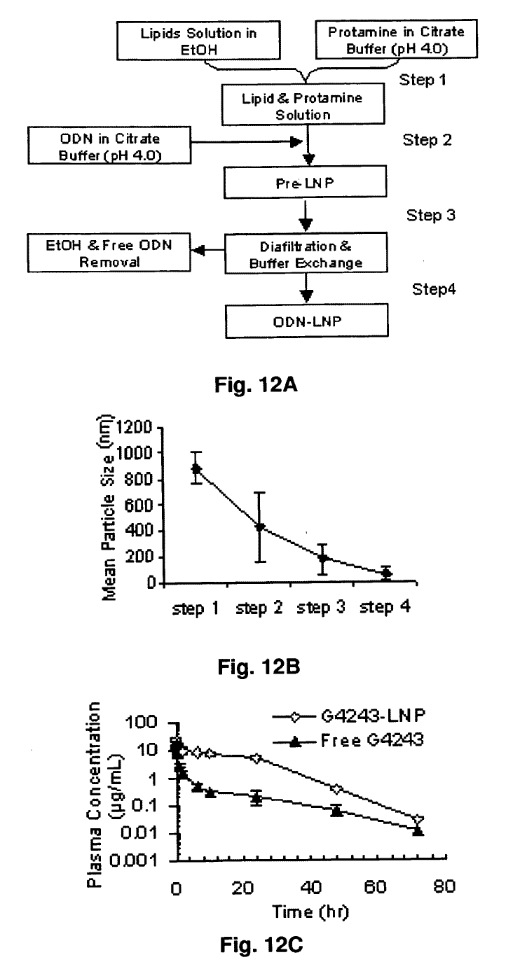

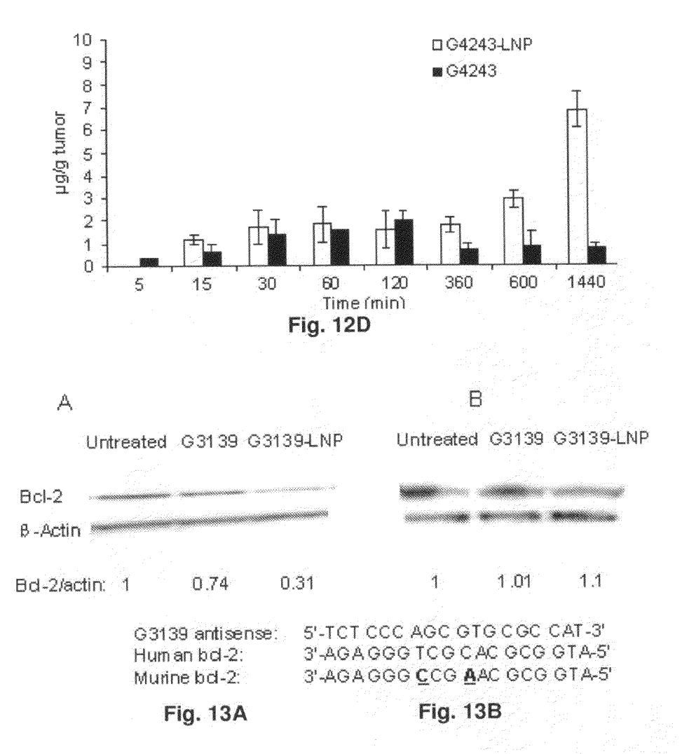

Preparation of Tf-conjugated G3139-containing LNs (Tf-LNs). The ethanol dilution method illustrated in FIG. 12A was used for the synthesis of LNs containing G3139. A lipid mixture egg PC/DC-Chol/PEG₂₀₀₀-DSPE at molar ratios of 65/30/5 was dissolved in ethanol (EtOH), and then mixed with protamine in a citrate buffer (20 mM, pH 4) at ratios for lipid:protamine of 12.5:0.3 (w/w) and EtOH:water of 2:1 (v/v). G3139 was dissolved in citrate buffer (20 mM, pH 4) and then added into the lipid/protamine solution using a vortexing process to form “pre-LNs complexes” at an EtOH concentration of 40% (v/v).

The pre-LN complexes were then dialyzed against citrate buffer (20 mM, pH 4) at room temperature for 2 hours and then against HEPES-buffered saline (HBS, 20 mM HEPES, 145 mM NaCl, pH 7.4) overnight at room temperature, using a MWCO 10,000 Dalton Spectra/Por Float-A-Lyzer (Spectrum Labs, Rancho Dominguez, Calif.) to remove free G3139 and to adjust the pH to the physiological range.

A post-insertion method was used to incorporate lipid-conjugated Tf ligand into G3139-loaded LNs. Briefly, holo-(diferric)Tf in HEPES-buffered saline (HBS, pH 8, containing 5 mM EDTA) was reacted with 5× Traut's reagent to yield holo-Tf-SH. Free Traut's reagent was removed by dialysis using a MWCO 10,000 Dalton Float-A-Lyzer and against MS. Holo-Tf-SH was coupled to micelles of Mal-PEG₂₀₀₀-DSPE at a protein-to-lipid molar ratio of 1:10. The resulting Tf-PEG₂₀₀₀-DSPE micelles were then incubated with the G3139-loaded LNs for 1 hour at 37° C. at Tf-PEG₂₀₀₀-DSPE-to-total lipid ratio of 1:100. For synthesis of fluorescence-labeled LNs, G3139 was spiked with 10% fluorescent oligonucleotide FITC-G3139. As a reference control, protamine-free liposomal G3139 (Lip-G3139) and Tf-Lip-G3139 were also prepared using essentially the same procedure except for omission of protamine from the formulation and an increase in DC-Chol content to maintain the overall cationic/anionic charge ratio.

The number of bound Tf per LN (molecules per vesicle) was calculated on the basis of the equation (A/B)C, where A, B and C represent the total number of Tf molecules in a LN suspension, the total number of lipid molecules in a LN suspension, and the number of lipid molecules per LN, respectively. The particle size of Tf-LNs was analyzed on a NICOMP Particle Sizer Model 370 (Particle Sizing Systems, Santa Barbara, Calif.). The zeta potential (ξ) of the LNs was determined on a ZetaPALS (Brookhaven Instruments Corp., Worcestershire, N.Y.). All measurements were carried out in triplicates.

G3139 entrapment efficiency. G3139 concentration was determined by dissolution of the LNs using 0.5% SDS followed by fluorometry to determine fluorescence derived from FITC-G3139, using excitation at 488 nm and emission at 520 nm. G3139 concentration was calculated based on a standard curve of fluorescence intensity versus oligonucleotide concentration. Loading efficiency of G3139 in the LNs was calculated based on the ratio of G3139 concentration in the LN preparation before and after dialysis.

Cryogenic transmission electron microscopy (cryo-TEM). Vitrified specimens for cryo-TEM imaging were prepared in a controlled environment vitrification system (CEVS) at 25° C. and 100% relative humidity. A drop of the liquid to be studied was applied onto a perforated carbon film, supported by a copper grid and held by the CEVS tweezers. The sample was blotted and immediately plunged into liquid ethane at its melting point (−183° C.). The vitrified sample was then stored under liquid nitrogen (−196° C.) and examined in a Philips CM120 YEM microscope (Eindhoven, The Netherlands), operated at 120 kV, using an Oxford CT-3500 cooling-holder (Abingdon, England). Specimens were equilibrated in the microscope at about −180° C., examined in the low-dose imaging mode to minimize electron beam radiation damage, and recorded at a nominal underfocus of 4-7 μm to enhance phase contrast. Images were recorded digitally by a Gatan 791 MultiScan CCD camera, and processed using the Digital Micrograph 3.1 software package. Further image processing was performed using the Adobe PhotoShop 5.0 package.

Colloidal and serum stability of Tf-LNs. The colloidal stability of Tf-LNs was evaluated by monitoring changes in the mean particle diameter during storage at 4° C. To evaluate the ability of the Tf-LNs to retain G3139 and protect it against nuclease degradation, the formulation was mixed with FBS at a 1:4 (v/v) ratio and incubated at 37° C. At various time points, aliquots of each sample were loaded onto a urea-polyacrylamide gel (Invitrogen). Electrophoresis was performed and G3139 bands were visualized by SYBR Gold (Invitrogen) staining. The densities of G3139 band were measured and analyzed by the ImageJ software.

Cellular uptake of Tf-LN G3139. Cellular uptake of Tf-targeted LNs and non-targeted control LNs, loaded with G3139 spiked with 10% fluorescent oligonucleotide FITC-G3139, was evaluated in MV4-11 cells. For the studies, 4×10⁵ cells were incubated with 1 μM G3139 entrapped in Tf-LNs at 37° C. After 4-hour incubation, the cells were washed three times with PBS, by pelleting of the cells at 1,000×g for 3 minutes, aspiration of the supernatant, followed by re-suspension of the cells in PBS. The cells were examined on a Nikon fluorescence microscope (Nikon, Küsnacht, Switzerland), or stained by 4′,6-diamidino-2-phenylindole (DAPI), a nuclear counterstain, and then examined on a Zeiss 510 META Laser Scanning Confocal microscope (Carl Zeiss Inc., Germany). G3139 uptake in leukemia cells was measured by flow cytometry on a FACSCalibur flow cytometer (Becton Dickinson, Franklin Lakes, N.J.).

Measurement of TfR expression on cell surface. TfR expression levels in leukemia cell lines were analyzed based on cellular binding of FITC-Tf determined by flow cytometry. Briefly, 4×10⁵ leukemia cells were washed with RPMI media containing 1% BSA and then incubated with 200 μg/ml FITC-Tf at 4° C. for 30 minutes. The cells were then washed twice with cold PBS (pH 7.4) containing 0.1% BSA, by pelleting of the cells at 1,000×g for 3 minutes, aspiration of the supernatant, followed by re-suspension of the cells in PBS. Finally, cellular fluorescence was then measured by flow cytometry.

Transfection studies. Leukemia cells were plated in 6-well tissue culture plates at 10⁶/well in RPMI 1640 medium containing 10% FBS. An appropriate amount of Tf-LNs or control formulations was added into each well to yield a final G3139 concentration of 1 μM. After 4-hour incubation at 37° C. in a CO₂ incubator, the cells were transferred to fresh medium, incubated for another 48 hours, and then analyzed for Bcl-2 mRNA level by real-time RT-PCR, for Bcl-2 protein level by Western blot, and for apoptosis by measuring caspase-9 activity, respectively.

Quantification of Bcl-2 mRNA level by Real-time RT-PCR. The bcl-2 mRNA level in leukemia cells was evaluated using real time RT-PCR, as previously described.²⁷ Briefly, total RNA was extracted using Trizol reagent (Invitrogen) and cDNA was synthesized by incubating RNA with random hexamer primer (Perkin Elmer, Boston Mass.), and then with reverse transcriptase (Invitrogen), reaction buffer, dithiothreitol, dNTPs and RNAsin, followed by incubation at 42° C. for 60 minutes and 94° C. for 5 minutes in a thermal cycler (Applied Biosystems, Foster City, Calif.). The resulting cDNA was amplified by real-time PCR (ABI Prism 7700 Sequence Detection System, Applied Biosystems) using bcl-2 primers and probes (forward primer CCCTGTGGATGACTGAGTACCTG [SEQ ID NO:2]; reverse primer CCAGCCTCCGTTATCCTGG [SEQ ID NO:3]; probe CCGGCACCTGCACACCTGGA [SEQ ID NO:4]). Housekeeping gene ABL mRNAs were also amplified concurrently and to which Bcl-2 mRNA were normalized.

Quantification of Bcl-2 protein by Western blot. Western blot was carried out. Briefly, untreated and G3139-treated cells were harvested at 24 or 48 hours after transfection and whole cellular lysates were prepared by lysing the cell in 1× cell lysis buffer containing a protease inhibitor cocktail (CalBiochem, San Diego, Calif.). Approximately 20 μg of cellular protein was used for immunoblotting using a monoclonal murine anti-human Bcl-2 (Dako, Carpinteria, Calif.) antibody. Bcl-2 protein expression levels were quantified by ImageJ software and were normalized to the β-actin levels from the same samples.

Analysis of apoptosis by caspase activation. To analyze cellular apoptosis, caspase-9 activities were measured on untreated and Tf-LN-G3139-treated cells using the caspase Glo-9 assay kit (Promega). Briefly, 5×10³ cells were plated in a white-walled 96-well plate, and the Z-DEVD reagent, a luminogenic caspase-9 substrate, was added with a 1:1 ratio of reagent to cell solution. After 90 minutes at room temperature, the substrate was cleaved by activated caspase-9, and the intensity of a luminescent signal was measured by a Fluoroskan Ascent FL luminometer (Thermo Electron Corp.). Differences in caspase-9 activity in Tf-LN-G3139-treated cells compared with untreated cells were determined by fold-change in luminescence.

Statistical analysis. Data obtained were represented as mean±standard deviations (S.D.). Comparisons between groups were made by 2-tailed Student's t-tests using the MiniTAB software (Minitab Inc., State College, Pa.). p<0.05 was used as the cutoff for defining statistically significant differences.

### Results for Example A

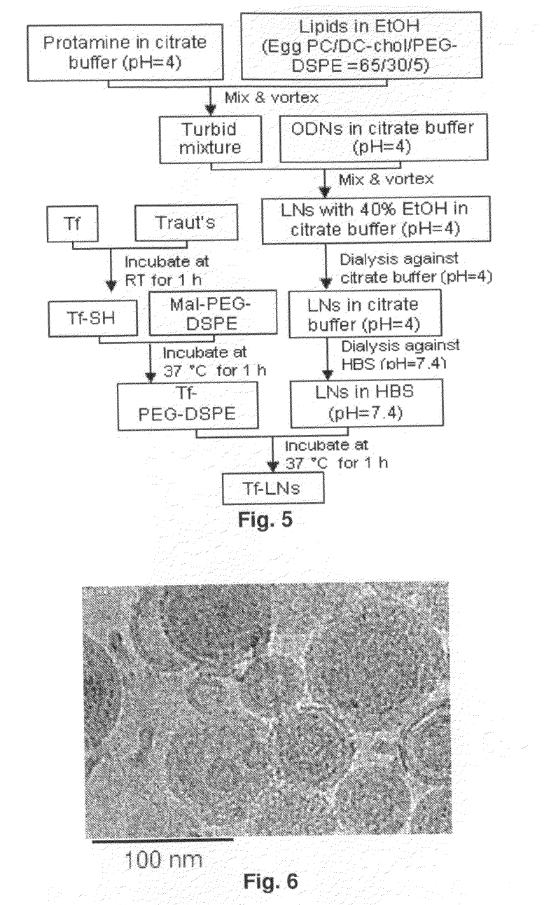

Physical chemical properties of the Tf-LNs. In order to increase the efficiency and specificity of G3139 delivery, Tf-LNs were synthesized. FIG. 5 shows the ethanol dilution method used for Tf-LN synthesis and the post-insertion of the Tf ligand.

Particle size values, zeta potential values, and G3139 entrapment efficiencies of LN formulations are presented in Table 3. The particle size and zeta potential of LNs with protamine were 78.1 nm and 5.7 mV and those of G3139-entrapping liposomes without protamine (Lips) were 112.5 nm and 2.0 mV, respectively. This showed that addition of protamine into the formulation resulted in a reduced particle size. Incorporation of Tf into LNs by post-insertion increased the particle size to 90.2 nm but did not significantly alter the zeta potential. The density of Tf on the resulting Tf-LN was estimated to be ˜46 Tf molecules per particle. The G3139 entrapment efficiencies of the formulations were also determined. The G3139 entrapment efficiency of LN and Tf-LN were 95.9±0.1% and 90.4±0.7%, respectively. These values were significantly greater than those for Lips and Tf-Lips without protamine, which were 76.1±0.2% and 71.9±1.1%, respectively. These results indicated that the incorporation of protamine in the formulation also increased the G3139 entrapment efficiency, whereas the insertion of Tf had only a minor effect on the G3139 entrapment efficiency.

TABLE 3
Particle size distribution, zeta potential, and G3139
entrapment efficiency of various formulations^{a}

| | Particle | Zeta | Entrapment |
|---|---|---|---|
| | size | potential | efficiency |
| | (nm) | (mV) | (%) |
| LN | 78.1 ± 3.4 | 5.7 ± 0.1 | 95.9 ± 0.1 |
| Tf-LN | 90.2 ± 3.6^{b} | 5.5 ± 0.6 | 90.4 ± 0.7^{b} |
| Lip | 112.5 ± 4.9^{b} | 2.0 ± 0.2^{b} | 76.1 ± 0.2^{b} |
| Tf-Lip | 132.5 ± 4.2^{b} | 1.0 ± 0.4^{b} | 71.9 ± 1.1^{b} |

^{a}Data represent the mean ± SD;
^{b}p < 0.05 vs LN group

The morphology of Tf-LNs was determined by cryoTEM. As shown in FIG. 6, the LNs appeared as spherical particles containing one to several lamellae. Due to the affinity of the G3139s to the cationic lipid component, it was quite possible that they were bound to lipid bilayers and/or were sandwiched between adjacent lipid bilayers.

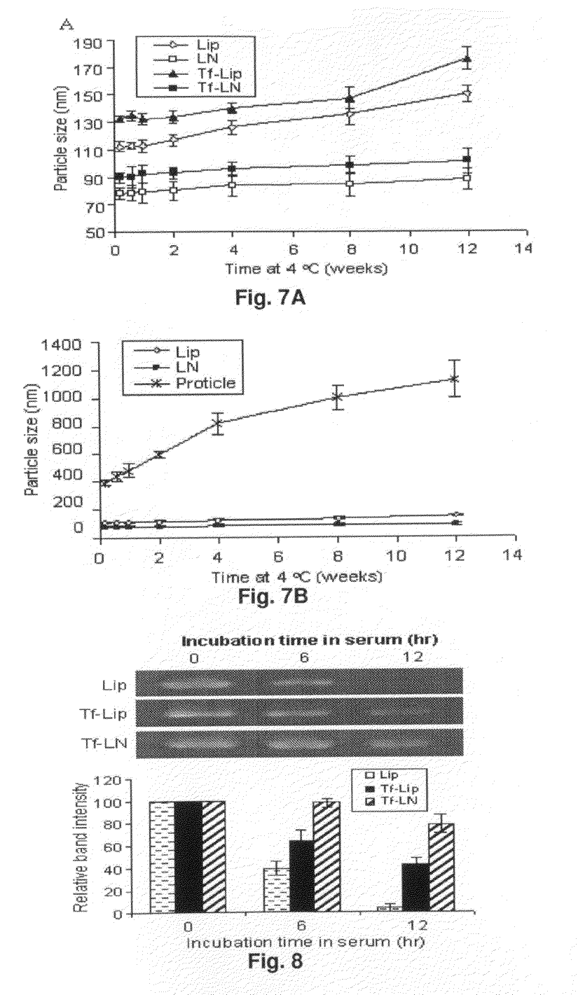

Colloidal and serum stability of Tf-LNs. The colloidal stability of G3139-loaded Tf-LNs was evaluated by monitoring changes in the mean diameter during storage in HBS buffer at 4° C. It was found that the LNs and Tf-LNs remained stable and no significant particle size changes were observed for 12 weeks at 4° C. (FIG. 7A). Meanwhile, Lips and Tf-Lips exhibited less colloidal stability with 32.6% and 33.6% increase in size in the same time period, respectively. In addition, protamine-G3139 complexes with the same protamine:ODN weight ratio of 3:1 but without the lipid components aggregated over time under the storage condition. These results indicated that the combination of lipids and protamine is required for colloidal stability of the nanoparticle formulation.

To evaluate the ability of the Tf-LNs both to retain G3139 and to protect it from degradation by nucleases, the formulations were incubated in FBS at 37° C. At various time points, samples were collected and analyzed by urea-polyacrylamide gel electrophoresis. As shown in FIG. 8, the amount of intact G3139 remaining in Tf-LN decreased slowly with incubation time. After 12 hours of exposure to serum, ˜80% of G3139 remained intact in Tf-LNs, whereas <10% of G3139 remained in the Lip formulation. Interestingly, Tf-Lips, although less stable in serum than Tf-LNs, retained 42% of loaded G3139 over the same incubation time frame.

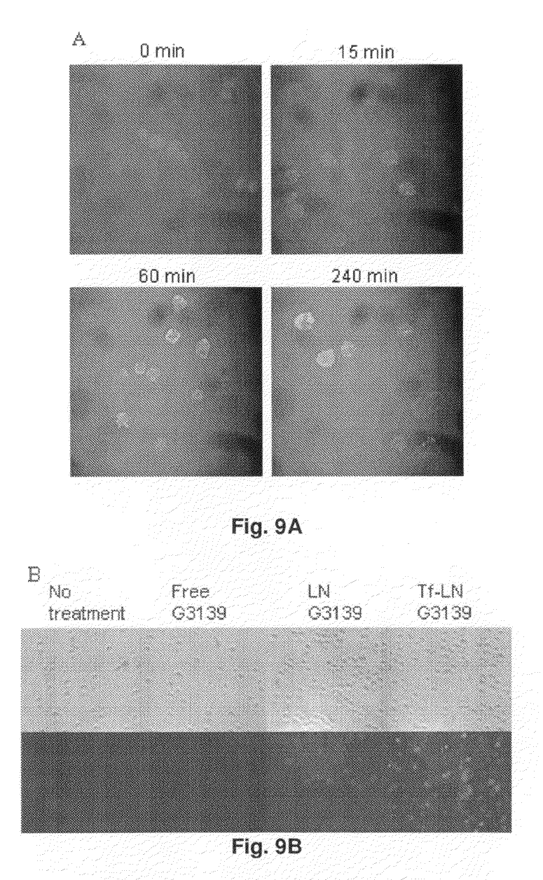

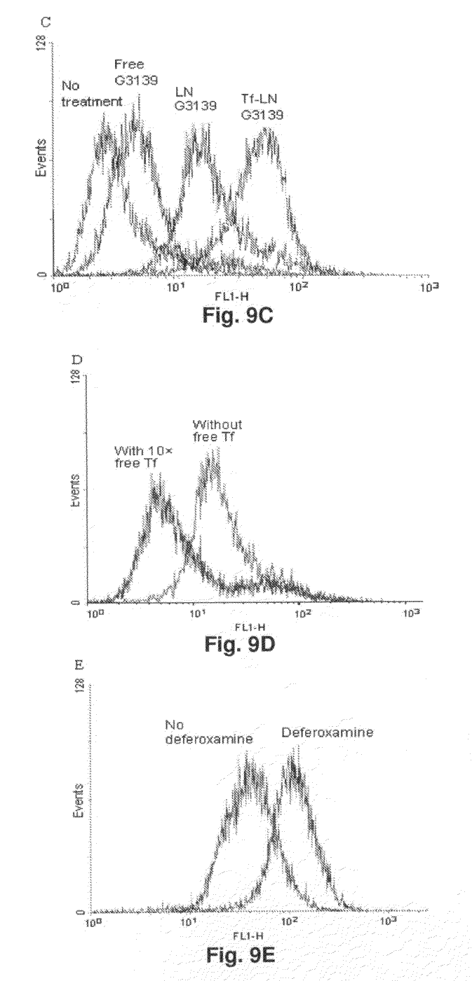

Cellular uptake of Tf-LN-G3139. Cellular uptake of Tf-LN-G3139, containing 10% fluorescent FITC-G3139, was evaluated in MV4-11 cells. By confocal microscopy, it was found that, after 15-minute incubation, most of the G3139 was bound to the cellular membrane. At 1 hour, the Tf-LNs were mostly internalized (FIG. 9 A).

Tf-LN G3139 was efficiently internalized by the cells and the level of uptake was much higher than that of free G3139 (FIG. 9B and FIG. 9C).

As a non-targeted control, delivery of G3139 via LNs was also evaluated. LN G3139 exhibited lower uptake compared to the Tf-LNs, showing that the enhancement of G3139 cellular uptake via Tf-LN was due to the presence of Tf ligands on the surface of LNs. In addition, Tf-LN mediated delivery was shown to be blocked by excess free holo-Tf (FIG. 9D), indicating that the increase in uptake was TfR specific. To investigate the role of TfR expression level in Tf-LN-G3139 cellular uptake, K562 cells were treated with 20 μM of deferoxamine, an iron chelator known to up-regulate cellular TfR expression, for 18 hours. These cells displayed a 3.3-fold higher level of Tf-LN-G3139 cellular uptake compared those that were untreated (FIG. 9E).

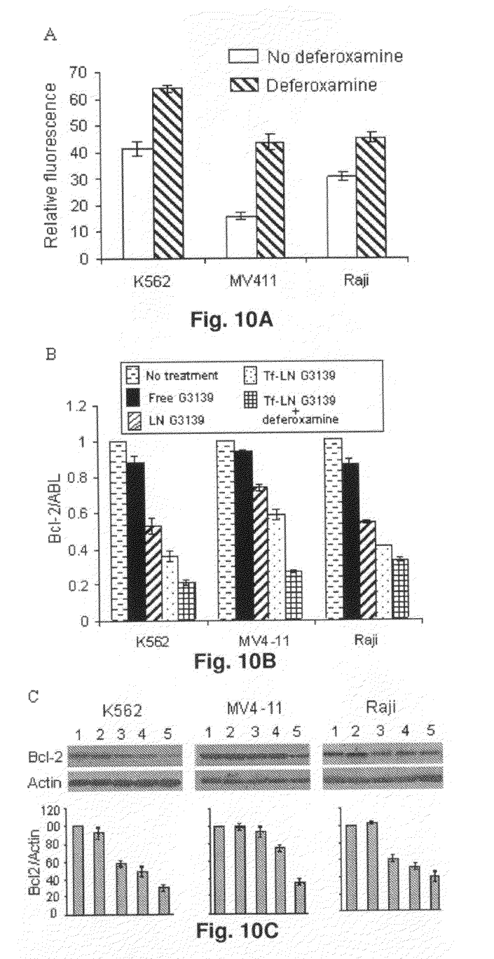

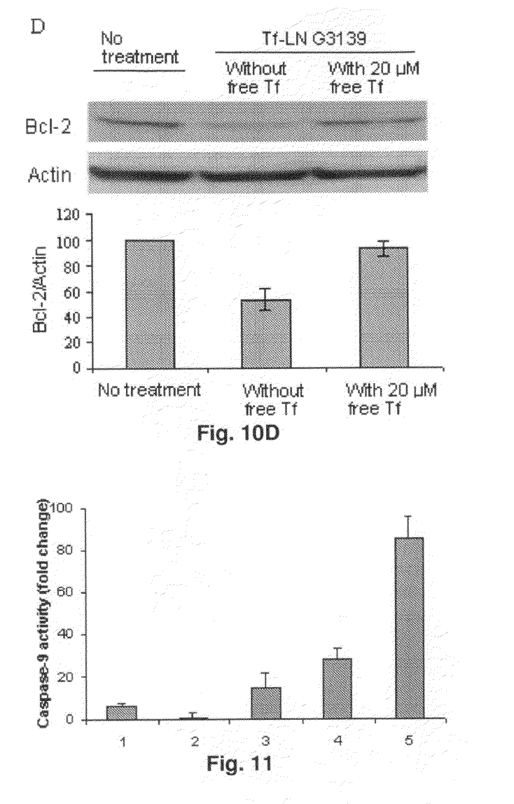

Tf-LN-G3139 mediated Bcl-2 down-regulation. TfR expression on leukemia cell lines K562, MV4-11 and Raji, with or without deferoxamine treatment are shown in FIG. 10A. TfR expression was increased upon deferoxamine treatment in all three cell lines. The leukemia cells were incubated with Tf-LN-G3139 for 48 hours. Real time RT-PCR and Western blot were performed for Bcl-2 mRNA level and protein expression determination, respectively. As shown in FIG. 10B, different cell lines had varied responses in Bcl-2 expression at the mRNA level. Bcl-2 mRNA reduction following treatment with Tf-LN-G3139 was 41% in MV4-11 cells compared to 26% following treatment with non-targeted LNs and 6% with free G3139. In K562 cells the Tf-LNs produced as high as 62% down-regulation of Bcl-2 at mRNA level, which was 2.2 times greater than that achieved by non-targeted LN. These demonstrated a more efficient down-regulation of Bcl-2 by the Tf-LNs. The same trend was observed based on the Bcl-2 protein level.

As shown in FIG. 10C, Tf-LN mediated the greatest reduction of Bcl-2 protein levels in all the cell lines studied compared to free G3139 and non-targeted LNs. For example, in K562 cells, Tf-LNs produced 54% down-regulation of Bcl-2 protein, which was 1.3 times and 50.2 times higher than that by non-targeted LN and free G3139, respectively. In addition, reductions in Bcl-2 expression by Tf-LNs were correlated with the TfR expression levels on cell surface. For example, K562 cells, which had the highest TfR expression levels among the studied leukemia cell lines (FIG. 10A), also showed the highest (65%) reduction in Bcl-2 at protein level. Interestingly, 20 μM free holo-Tf effectively blocked Bcl-2 down-regulation by Tf-LN-G3139 in K562 cells (FIG. 10D). This result indicated that Tf-LN mediated delivery of G3139 was dependent on TfR expression. Moreover, the increased TfR expression by deferoxamine in different leukemia cell lines (FIG. 10A) resulted in greater inhibition of Bcl-2 expression by Tf-LN-G3139 (FIG. 10B and FIG. 10C), further indicating that the delivery was TfR-dependent.

Tf-LNs containing G3139 exhibited pronounced effect on cell apoptosis. Having demonstrated knockdown of the anti-apoptotic protein Bcl-2, we next sought to determine the effect of Tf-LNs containing G3139 on cellular apoptosis. Leukemia K562 cells were treated with the Tf-LNs. We observed, by confocal microscopy, that G3139 accumulated inside the cells after 1-hour treatment. At 240 minutes, nuclei in some of the cells were fragmented, indicating the occurrence of apoptosis in these cells (FIG. 9A). At 48 hours after the treatment, the cells were collected and analyzed for caspase-9 activities. As shown in FIG. 11, caspase-9 activity in cells treated with Tf-LN-G3139 was 2× higher than in those treated by non-targeted LN and 43× higher than those treated by free G3139, indicating markedly enhanced apoptosis induction by the Tf-LNs. Pre-treatment of K562 cells by deferoxamine further increased caspase-9 activity to 3× that of untreated cells, suggesting that the enhanced apoptosis by Tf-LN-G3139 was TfR level-dependent.

### Discussion of Example A

TfR-targeted LNs exhibit colloidal stability and have high efficiency and selectivity in G3139 delivery to leukemia cells. The LNs incorporated both protamine and lipids. Tf was incorporated to provide TfR-mediated leukemia cell targeting. These nanoparticles were shown to efficiently deliver G3139 to TfR-positive leukemia cells, as shown by effective down-regulation of Bcl-2.

The lipid composition used in Example A was egg PC/DC-Chol/PEG₂₀₀₀-DSPE (molar ratio 65/30/5). The utilizations of both protamine and DC-Chol as positive charged components ensured high G3139 loading efficiencies. During LN assembly, G3139 was mixed with protamine and cationic lipids. The faster diffusion rate and charge density of protamine compared to Lips, allows the ODN to first interact with protamine, to form the pre-LN complexes, which resulting complexes are then stabilized by a further coating of the lipids to form the lipid oligonucleotide nanoparticles (LNs). The targeting ligand formed as micelles of Tf-PEG-DSPE, which are introduced by post-insertion, are then distributed on the surface of the nanoparticles. In this process, the micelles are disassembled and their components are incorporated into the bilayers of the LNs.

When the pH is adjusted to 7.5 upon removal of EtOH by dialysis, the head group of DC-Chol became partially deprotonated. The zeta potential of the resulting LNs following dialysis was low (5.7 mV).

The resulting LNs have excellent colloidal stability, which is believed by the inventors herein to be due to the high DNA binding activity of protamine and surfactant characteristics of the lipids. In this example, the PEG₂₀₀₀-DSPE in the formulation provides steric stabilization of the LNs. Also, Tf conjugate may also contribute to LN stability in serum by shielding them from interactions with plasma proteins.

Pre-mixing of the complexing agent (here protamine) with the lipids provides the desired small particle formation. It is to be noted that G3139/protamine complexes in the absence of lipids undesirably aggregate over time. In addition, pre-mixing of protamine with G3139 and then adding this mixture into the lipids also resulted in unstable particles that aggregated over time. Using the process described herein, the G3139 encapsulation efficiencies were 95.9% and 90.4% for LN and Tf-LN, respectively. Therefore, the LN formulation is much superior to protamine-oligonucleotide and lipid-oligonucleotide complexes both in terms of DNA loading efficiency and colloidal stability.

To investigate G3139 delivery efficiency via Tf-LNs, Bcl-2 down-regulation was evaluated in 3 different leukemia cell lines (K562, MV4-11 and Raji), followed by the measurement of caspase-dependent apoptosis in K562 cells. TfR expression level was found to be an important factor in determining the efficiency of G3139 delivery by Tf-LNs. Deferoxamine, a clinically used iron chelator for the treatment of secondary iron overload, is known to up-regulate TfR expression in cells. Therefore, deferoxamine should increase TfR-targeting efficiency of the Tf-LNs. This was confirmed by the enhanced Bcl-2 down regulatory activities of the deferoxamine-treated leukemia cells by Tf-LNs. Positive correlation between Bcl-2 down-regulation by Tf-LN and enhancement of TfR expression by deferoxamine suggests a potentially promising novel strategy for enhancing delivery and therapeutic efficacy of antisense oligonucleotides.

Example A thus shows that a stable, TfR-targeted LN formulation encapsulating antisense G3139 exhibits excellent G3139 loading efficiency and colloidal stability and the G3139 is protected against degradation by serum nucleases. Tf-LNs showed efficient delivery of G3139 to TfR-positive leukemia cells, which can be blocked by excess free Tf. Deferoxamine treatment increased TfR expression and enhanced the transfection activity of Tf-LNs. Combining defeoxamine pretreatment with Tf-LN mediated delivery is a promising strategy for targeted delivery of G3139 and other antisense drugs to leukemia cells.

### Example B

In Example B, lipid nanoparticles (LNPs) encapsulating G3139 were synthesized and evaluated in mice bearing L1210 subcutaneous tumors. Intravenous injection of G3139-LNPs into mice led to increased serum levels of IL-6 and IFN-γ, promoted proliferation of natural killer (NK) cells and dendritic cells (DCs), and triggered a strong anti-tumor immune response in mice. The observed effects were much greater than those induced by free G3139. Correspondingly, the G3139-LNPs more effectively inhibited tumor growth and induced complete tumor regression in some mice. In contrast, free G3139 was ineffective in tumor growth inhibition and did not prolong survival of the tumor bearing mice. These results show that G3139-LNPs are a potential immunomodulatory agent and may have applications in cancer therapy.

### Introduction for Example B

The LNPs prolonged plasma half-life and tumor accumulation of G3139, showing that intravenously injected G3139-LNPs (rather than free G3139) can effectively activate innate immune system cells in a way that results in a potent anti-tumor immune response and tumor growth inhibition.

### Materials and Methods for Example B

Materials. 313-[N,N-(Dimethylaminoethane)-carbamoyl]-cholesterol (DC-Chol), egg yolk phosphatidylcholine (PC), and distearoylphosphatidylethanolamine-N-[methoxy(polyethylene glycol)-2000] (m-PEG₂₀₀₀-DSPE) were purchased from Avanti Polar Lipids (Alabaster, Ala.). Protamine sulfate was purchased from Sigma Chemical Co. (St. Louis, Mo.). 5-Bromo-2′deoxyuridine (BrdU) Flow Cytometry Assay kit was obtained from BD Pharmingen (San Diego, Calif.).

Oligonucleotides G3139 (5′-TCT CCC AGC GTG CGC CAT-3′) [SEQ ID NO:1], G4243 (FAM-G3139, with a 5′-fluorescein label), and control ODNs G4126 (5′-TCT CCC AGC ATG TGC CAT-3′) [SEQ ID NO:5] (2 nucleotides different from G3139 and containing no CpG motifs).

Phycoerythrin (PE)-, fluorescein isothiocyanate (FITC)-, Allophycocyanin (APC)-, and (PE-Cy7)-conjugated monoclonal antibodies (mAbs), including PE-Cy5.5-CD4, APC-CD8, APC-NK-DX5, PE-CD3e, PE-INF-7 were purchased from BD Pharmingen (San Diego, Calif.). Anti-CD112 and anti-CD40 MAbs were purchased from BioExpress (West Lebanon, N.H.).

Cell culture. Human KB cell line, which has been identified as a subline of human cervical cancer HeLa cell line, was obtained as a gift from Dr. Philip Low (Purdue University, West Lafayette, Ind.). L1210, a murine lymphocytic leukemia cell line, were kindly provided by Dr. Manohar Ratnam (University of Toledo, Toledo, Ohio). Cells were cultured in RPMI 1640 medium supplemented with 100 units/mL penicillin, 100 μg/mL streptomycin, and 10% FBS in a humidified atmosphere containing 5% CO₂ at 37° C.

Preparation of ODN-Lipid nanoparticles (ODN-LNPs). LNPs, composed of DC-Chollegg PC/mPEG₂₀₀₀-DSPE (35:60:5, mole/mole), protamine and ODN, were prepared by EtOH dilution followed by tangential flow diafiltration (FIG. 12A). The lipids were dissolved in EtOH and mixed with protamine sulfate in citrate buffer (20 mM Na citrate, pH 4.0) to achieve a lipid:protamine weight ratio of 25 and a final EtOH concentration of 66.6% (v/v). ODN in citrate buffer (20 mM, pH 4.0) was then added to the lipids/protamine solution to form pre-LNPs at an EtOH concentration of 40% (v/v). The pre-LNPs were then diluted with 20 mM citrate buffer (pH 4.0) to further lower the EtOH concentration, and then were subjected to diafiltration in a Millipore lab-scale tangential flow filtration (TFF) unit (Billerica, Mass.) to remove excess EtOH and unencapsulated ODN. Finally, the resulting LNPs were buffer-exchanged into HBS (150 mM NaCl, 20 mM HEPES, pH 7.5). Empty LNPs of the same lipid composition but containing no ODN were also prepared by the same procedure.

The particle size of the LNPs was determined by dynamic light scattering via Nicomp model 370 Submicron Particle Sizer (Particle Sizing Systems, Santa Barbara, Calif.). The zeta potential (ξ) of the LNPs was measured on a Brookhaven 90plus Particle Analyzer (Holtsville, N.Y.).

To evaluate ODN encapsulation, FITC labeled G3139 (G4243) was used instead of G3139 to enable fluorometric measurements of ODN concentration. To determine ODN content, LNPs were lysed by 1% SDS at 95° C. for 5 min and were centrifuged at 12,000×g for 5 min. The ODN concentration in the LNPs was determined by measuring fluorescence value obtained from supernatant of LNP lysate with a spectrofluorometer (Perkin-Elmer) at excitation and emission wavelengths of 495 and 520 nm, respectively, based on a pre-established standard curve. Encapsulation efficiency was calculated based on ODN concentration in the lysate divided by ODN concentration added.

Western blot for Bcl-2. The Bcl-2 downregulatory effect of G3139-LNPs was evaluated in KB and L1210 cells. Cells were treated by lysis buffer 72 hr after treatment. From the lysate 100 μg proteins was loaded on a 15% SDS-PAGE gel (Bio-Rad, Hercules, Calif.) and run for 2 hr at 100 V, followed by transferring to a nitrocellulose membrane overnight. After blocking with 5% non-fat milk in Tris-buffered saline/Tween-20 (TBST) for 2 hr, the membranes were incubated with murine anti-human Bcl-2 antibody (Dako, Carpinteria, Calif.) for studies on KB cells or hamster anti-mouse Bcl-2 antibody (BD Pharmingen, San Diego, Calif.) for studies on murine L1210 cells, respectively. After 2 hr of incubation at room temperature, membranes were the treated with horseradish peroxidase-conjugated sheep anti-mouse IgG antibody (GE Health, Piscataway, N.J.) for KB cell or murine anti-hamster IgG antibody (BD Pharmingen, San Diego, Calif.) for L1210 cell for 1 hr at room temperature. Membranes were then developed with Pierce SuperSignal West Dura Extended Duration Substrate (Pierce, Rockford, Ill.) and imaged with Kodak X-OMAT film (Kodak, Rochester, N.Y.). Bcl2 protein expression levels were quantified by ImageJ software (NIH Image, Bethesda, Md.) and normalized to the β-actin levels from the same samples.

In vivo assay for plasma clearance and tumor accumulation of ODN-LNP. Female DBA/2 mice (H-2^{d}), 8 wks old, were purchased from Harlan (Indianapolis, Ind.). To evaluate in vivo plasma clearance and tumor accumulation of ODN-LNPs, G4243 (fluorescein labeled G3139) or G4243-LNPs were administered at 5 mg/kg ODN dose by tail vein injection. At appropriate time points, mice were anesthetized and blood was collected via the tail vein and into heparinized tubes. Plasma was separated from red blood cells via immediate centrifugation at 1000×g for 5 min. Mice were sacrificed by carbon dioxide asphyxiation. Tumors were harvested at various time points and homogenized in microtubes containing 500 μL distilled water. Samples were then treated with 1% SDS, and heated at 95° C. for 5 min, followed by centrifugation at 12,000×g for 5 min. The fluorescence of supernatant was determined by spectrofluorometry to determine sample ODN concentration, as described above. WinNonlin Version 3.2 (Pharsight Co., CA) was used to determine pharmacokinetic parameters, including area under the curve (AUC), total body clearance (CL) and plasma half-life.

Cytokine production and cell proliferation. To determine serum INF-γ and IL-6 levels, blood was collected from the tail vein of mice at various time points after i.v. injection of G3139-LNPs, free G3139, empty LNPs, or non-CpG containing G4126-LNPs. Three mice were used in each treatment group. The blood samples were kept at room temperature for 30 min and then centrifuged at 12000×g to harvest serum. The levels of cytokines were determined by ELISAs using commercial kits (BD Pharmingen, San Diego, Calif.).

In vivo immune cell proliferation was evaluated by BrdU incorporation assay. BrdU (10 mg/mL) was injected i.p. into mice at 1 or 6 days after treatment. Three mice were used in each group. Splenocytes were harvested from the mice 24 hr after the BrdU administration, and were surface-stained using fluorescence-labeled mAbs to CD4, CD8, CD3 and/or CD49b (DX5), followed by intracellular staining with mAb to BrdU as instructed by the manufacturer (BD Biosciences). Then the cells were washed twice in perm/wash solution and were resuspended in 300 μL of FACS buffer for flow cytometry analysis. Data were acquired on a Becton Dickinson FACSCalibur (Becton Dickinson) and analyzed using the FlowJo software (Tree Star, Ashland, Oreg.). In a typical assay, 100,000 events were acquired for analysis.

Histopathological and immunohistochemical (IHC) analyses. For pathological analysis, tumor samples were fixed in 10% phosphate buffered formalin solution. The tissue sections were stained with hematoxylin and eosin (H&E). For IHC analysis, tumor samples were frozen and prepared as described previously. Briefly, samples were fixed and washed with ice-cold PBS (pH 7.4) and stained with rat mAbs against CD4, CD8, or CD122, (2 μg/mL in PBS for 1 hr at 4° C.) followed by staining with horseradish peroxidase-conjugated rabbit anti-rat IgG.

Evaluation of anti-tumor activity. L1210 cells (5×10⁶) were subcutaneously inoculated into the flank of syngeneic DBA/2 mice. Palpable tumors developed within 4-5 days after inoculation. At 7 days post inoculation, the tumor-bearing mice were injected i.v. with PBS (pH 7.4), free ODN (G3139), empty LNPs, G3139-LNPs or non-CpG containing G4126-LNPs (1.5 mg/kg or 5 mg/kg dose of ODN) on every 4th days (Q4D). Five mice were used in each treatment group. Anti-tumor activity was determined by measuring the tumor size (width and length) using a Vernier caliper at a series of time points. Tumor volume was calculated by the formula: tumor volume=(n16)×length (mm)×[width (mm)]². Mice were sacrificed once the tumor size reached greater than 1500 mm³.

Statistical analysis. Statistical analysis was performed with Analysis of Variance (ANOVA) or Student's t test and by JMPT™ software, where appropriate. Differences in survival of mice among treatment groups were analyzed using the log-rank test. A p value of <0.05 was considered significant.

### Results for Example B

LNPs showed prolonged plasma half-life and increased G3139 accumulation in tumors. G3139-LNPs and G4243-LNPs were prepared by the EtOH dilution/diafiltration method. At a high EtOH concentration, the lipids form a metastable bilayer structure, which enables efficient ODN loading in the nanoparticle. In the subsequent dilution and diafiltration steps, EtOH concentration is gradually decreased, thus resulting in a “sealing off” of the lipid bilayers. The particle sizes changed with EtOH concentration in each step (FIG. 12B).

After removal of excess EtOH, the protocol yielded small ODN-LNPs with a mean diameter of 89±45.6 nm, encapsulation efficiency of >95%, and zeta potential of 4.08±0.4 mV. G3139-LNPs and G4243-LNPs had essentially identical characteristics.

The circulation time of LNP-encapsulated ODNs was evaluated by measuring plasma clearance of G4243-LNPs (G4243 is fluorescein-labeled G3139) in L1210 tumor bearing DBA/2 mice. At 24 hr after intravenous administration, ˜25% of the injected G4243-LNPs remained in the plasma, yielding a plasma half-life of about 8 hr (FIG. 12C). In contrast, only 1% of the free G4243 was detected in the plasma 24 hr after the i.v. injection, yielding a plasma half-life of about 45 min. Thus, the circulation time of G4243 was extended by >10 times when incorporated into LNPs. Plasma concentration versus time data were analyzed by WinNonLin using non-compartmental model to determine pharmacokinetic parameters. As shown in Table 4, i.v. administration of G4243-LNPs resulted in a terminal elimination half-life (T_{1/2}) of 0.47 h, area under the plasma concentration time curve (AUC) of 85.0 h˜μg/ml, volume at steady state (Vₛₛ) of 363.6 ml/kg and clerance (CL) of 58.9 ml/kg/h. In comparison, free G4243 had a much shorter T_{1/2} and 10-time increased CL.

TABLE 4
Pharmacokinetic parameters of G4243-LNPs and free
G4243 after i.v. bolus administration at 5 mg/kg^{a}

| | T_{1/2} | Vₛₛ | AUC | CL |
|---|---|---|---|---|
| | (h) | (ml/kg) | (h · μg/ml) | (ml/kg/h) |
| G4243- | 0.47 (7.4%) | 363.6 (4.6%) | 85.0 (5.0%) | 58.9 (10.3%) |
| LNPS | | | | |
| Free | 0.08 (2.3%) | 105.0 (6.6%) | 8.7 (10.0%) | 577.4 (9.8%) |
| G4243 | | | | |

^{a}Data generated by WinNonlin. Standard errors were shown in parenthesis as (CV %)

These data show that the G4243-LNPs had a greatly prolonged blood circulation time and decreased elimination rate. The accumulation of G4243-LNPs in tumor tissue was also significantly enhanced. The G4243-LNPs level in tumor was at 6.9 μg ODN/g tumor tissue at 24 hr after i.v. bolus administration (FIG. 12D), whereas the free G4243 in the tumor tissue was 0.75 μg ODN/g tumor tissue. These results indicated that the LNP encapsulation could extend circulation time of ODNs as well as enhance accumulation of G4243 in the tumor tissue, possibly due to enhanced permeability and retention (EPR) effect.

G3139-LNPs did not induce Bcl-2 down-regulation in murine L1210 Cells. G3139 is an antisense ODN designed for targeting the human Bcl-2. Against murine Bcl-2, G3139 has a two nucleotides mismatch. The effects of G3139 on Bcl-2 expression were evaluated in human KB and in murine L1210 cells. The cells were incubated with either G3139 or G3139-LNPs for 72 hrs and were harvested for Western-blot analysis of Bcl-2 protein expression. As shown in the FIGS. 13A-13B, while both free G3139 and G3139-LNPs significantly inhibited Bcl-2 expression in human KB cells (FIG. 13A), they had no significant effect on Bcl-2 expression in murine L1210 cells (FIG. 13B). These results suggested that Bcl-2 down-regulatory activity of G3139 is specific for human.

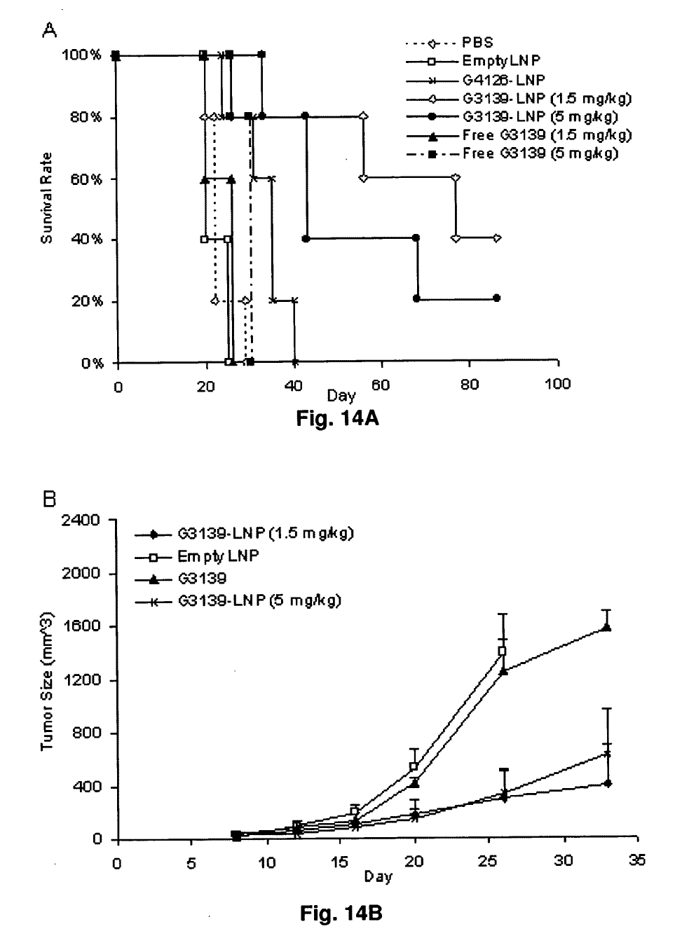

G3139-LNPs inhibited tumor growth. The G3139-LNPs were studied for therapeutic efficacy in mice with established solid tumors. A tumor model was established with DBA/2 mice, which were injected subcutaneously with syngeneic L1210 tumor cells. The mice developed solid tumors of ˜30 mm³ within 7 days, which reached sizes >1500 mm³ within 1 month in the absence of treatment. For the therapeutic study, the mice were injected i.v. with 100 μL of G3139-LNPs every 4 days started from day 7 post inoculation. The mice of control groups were injected i.v. with the same volume of PBS (pH 7.4), empty LNPs, free G3139, or non-CpG containing G4126-LNPs. As shown in FIGS. 14A-14B and Table 5, tumor growth in the mice treated with G3139-LNPs was inhibited by >50% (p<0.005), resulting in prolonged survival of 80% of the mice (⅘) with a median survival time (MST) of 76 days and increase-in-lifespan (ILS) value of 245% (p=0.002) and complete rejection of tumors in 40% (⅖) of the mice after 3 injections with 1.5 mg/kg (low dose) of G3139-LNPs.

TABLE 5
Survival of mice after treatments by
LNP-G3139s and other formulations

| | Median | | Increase in | Log-rank p |
|---|---|---|---|---|
| | survival | T/C | lifespan | compared to |
| Formulation | time (days) | (%) | (%) | PBS group |
| PBS | 22 | 100 | 0 | |
| Empty LNP | 20 | 91 | −9 | 0.3 |
| Free G3139 (1.5 mg/kg) | 25 | 114 | 14 | 0.3 |
| Free G3139 (5 mg/kg) | 30 | 136 | 36 | 0.1 |
| G4126 LNP | 35 | 159 | 59 | 0.03 |
| LNP-G3139 (1.5 mg/kg) | 76 | 345 | 245 | 0.002 |
| LNP-G3139 (5 mg/kg) | 43 | 195 | 95 | 0.01 |

(n = 5 for each treatment group)

In contrast, the mice treated with free G3139 (1.5 mg/kg) did not respond. For this group, the tumor size were comparable to the mice treated with PBS, empty LNPs, or G4126-LNPs (FIG. 14A) and the ILS value was not significantly different from the PBS control group (p=0.1). In fact, neither G3139 nor empty LNPs had a significant effect on tumor growth (FIGS. 14A-14B). Moreover, the antitumor effect of G3139 was likely mediated by CpG motif, because G4126-LNPs, which lacked CpG motifs, did not inhibit tumor growth (FIG. 14B).

To determine whether the antitumor effect of G3139-LNPs was dose-dependent, we treated tumor-bearing mice with either 1.5 mg/kg (low dose) or 5 mg/kg (high dose) of G3139-LNPs or free G3139. Neither dosing levels of free G3139 produced antitumor activities (FIG. 3A). As shown in FIG. 14 and Table 5, high dose of G3139-LNPs (5 mg/kg) did not result in a better therapeutic effect compared to low dose (1.5 mg/kg) G3139-LNPs. The median survival was actually decreased from 76 days to 43 days, and only one mouse had complete tumor eradication. Thus, the experiments described below used only low dose of G3139-LNPs.

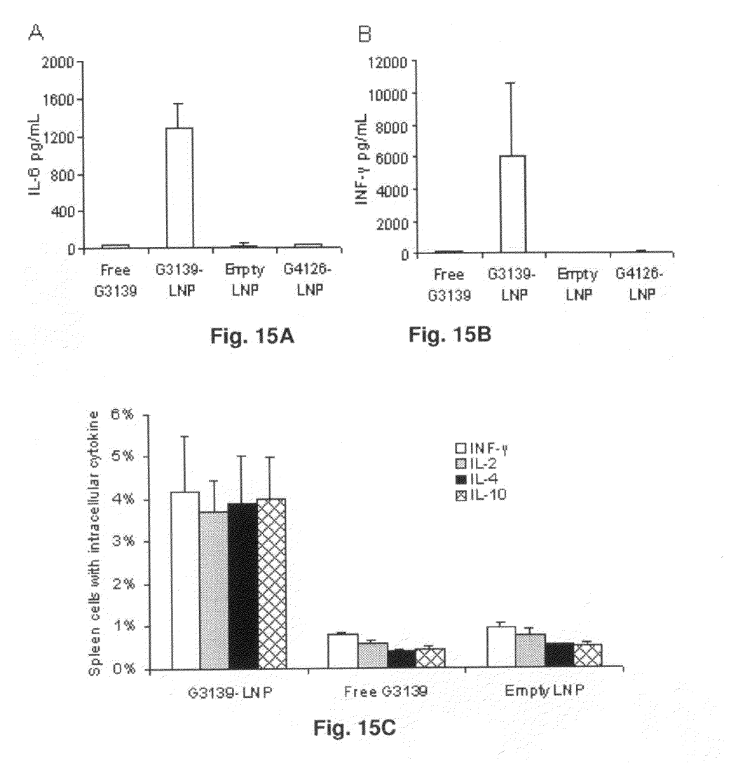

G3139-LNPs potently activated innate immune system Cells. Since CpG-ODNs stimulate innate immune responses, we examined cytokine production and innate immune cell proliferation in mice treated with G3139-LNPs. The levels of IL-6 and IFN-γ were evaluated in the peripheral blood because these are important for the induction of Th17 and ml responses, respectively. DBA/2 mice were injected i.v. with 1.5 mg/kg of G3139, G3139-LNPs, non-CpG containing G4126-LNPs or empty LNPs. The serum levels of IL-6 and IFN-γ were determined by ELISA after 4 and 8 hour of injection, respectively (FIGS. 15A-15B). The highest level of IL-6 was observed at 4 hr following intravenous injection of G3139-LNP, whereas the highest level of INF-γ was detected at 8 hr after injection. Only low levels of IL-6 or INF-γ were detected in the sera of mice treated with free G3139, non-CpG containing G4126-LNPs, or empty LNPs.

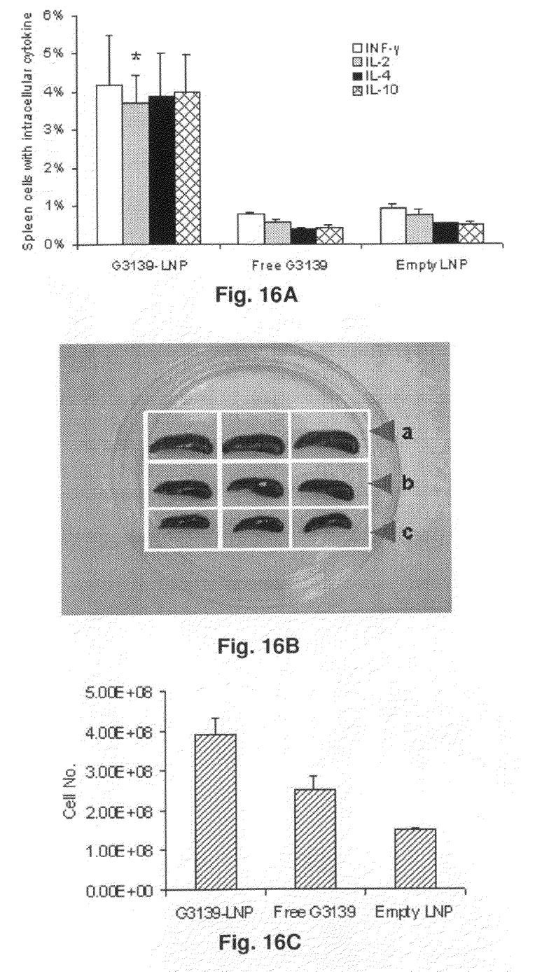

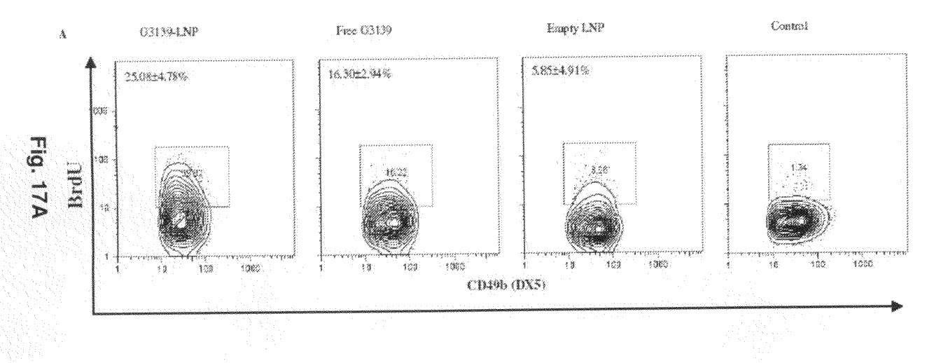

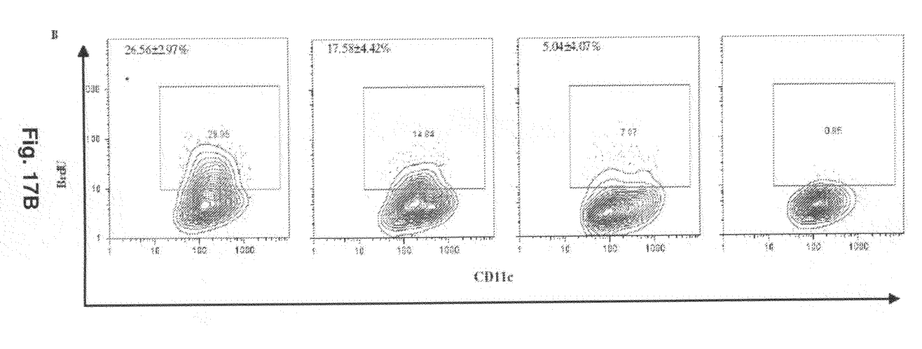

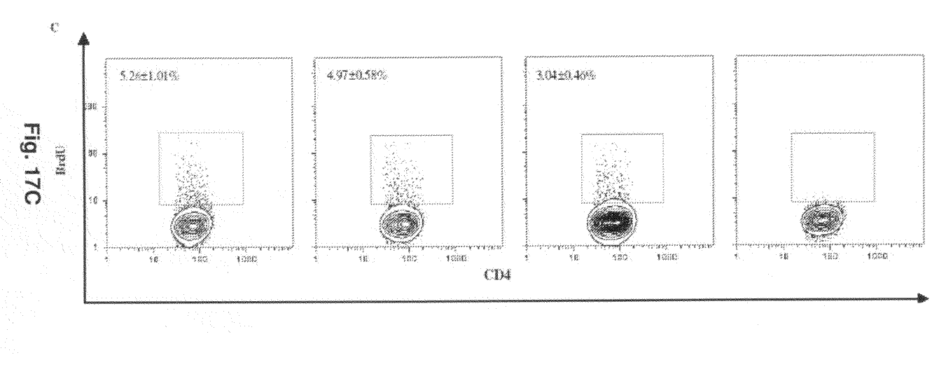

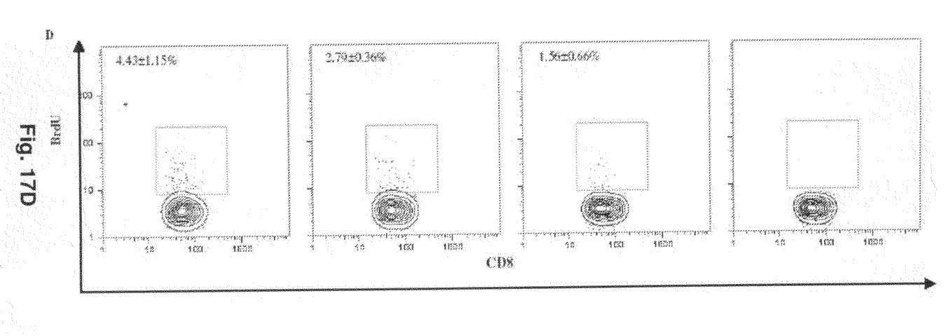

The splenocytes from the mice treated with G3139-LNPs produced more cytokines, including IFN-γ, IL-2, IL-4 and IL-10, than those treated with free G3139 or empty LNPs, as shown by immunohistochemical staining of spleen (FIG. 16A). These results show that the antitumor activity of G3139-LNPs may be associated with their high potency in cytokine induction.

In addition to cytokine production, G3139-LNPs also promoted immune cell proliferation. LNP-treated mice showed significantly enlarged spleens and increased spleen cells at 7 days after treatment. The effect was much more pronounced compared to in mice treated with G3139 (p=0.0017) or empty LNPs (p<0.0001) (FIG. 16B, FIG. 16C).

To verify that the expansion of the spleen cells was associated with proliferation of innate immune cells, such as NK and dendritic cells (DCs), we examined BrdU incorporation by these cells. BrdU, an analog of thymidine, can replace thymidine during cell division, and has been widely used for quantification of cell proliferation, especially in vivo. The mice bearing L1210 tumors were treated with G3139-LNPs, G3139 or LNPs for 2 days, and BrdU was administered i.p. The mice were then sacrificed 24 hrs later and analyzed.

As shown in FIGS. 17A-17D, LNPs alone had little effect on NK and DC expansion, at 5.85% and 5.05%, respectively. Also, free G3139 ODN had a significant effect on NK and DC proliferation, at 16.30% and 17.58%, respectively. The LNPs loaded with G3139 induced a much greater effect than free G3139 and empty LNPs on the expansion of NK and DCs (25.08% and 26.56%, respectively, p<0.05) (FIGS. 17A-17D). The studies were repeated twice, and produced similar results. These results indicated that G3139-LNPs not only elicited innate immune cells to produce cytokines, but also promoted their proliferation.

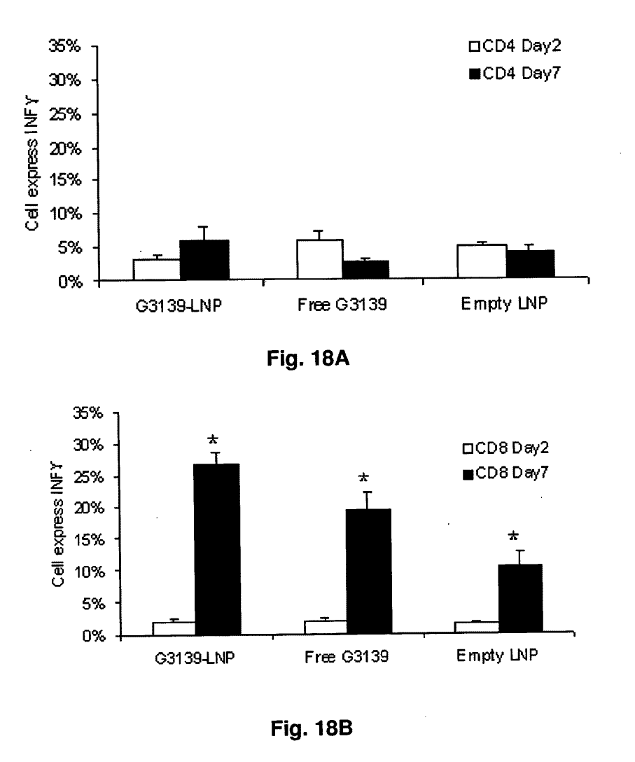

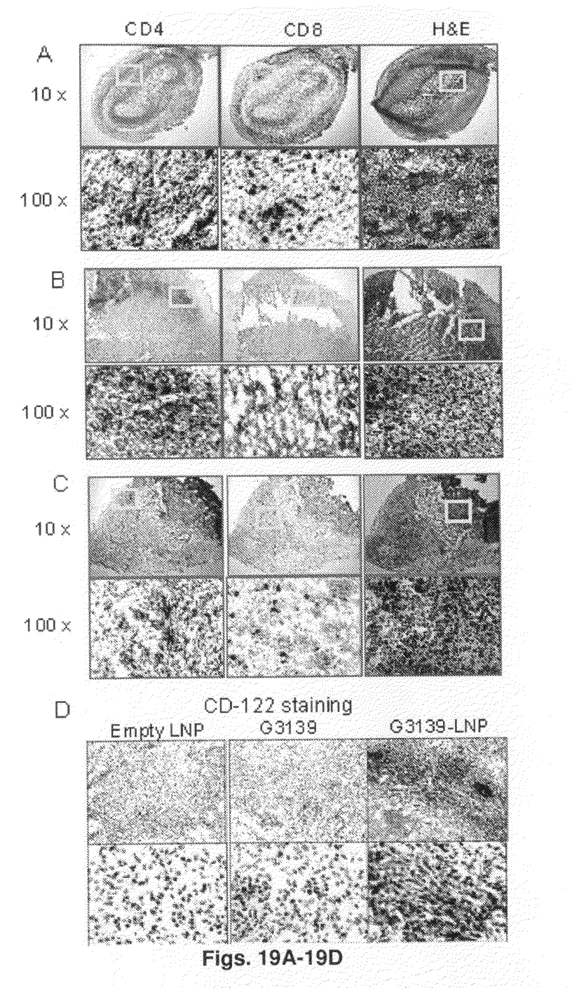

The effect of G3139-LNPs on adaptive anti-tumor immunity. Since activation of innate immune cells can induce adaptive immunity, we further characterized the adaptive immunity in the tumor-bearing mice treated with G3139-LNPs. Since the IFN-γ-mediated adaptive immune response is important for anti-tumor immunity, we examined IFN-γ-production by CD4⁺ and CD8⁺T cells in the spleen of the mice at day 2 and 7 after treatment. At day 2 post-treatment, IFN-γ-producing cells among CD4⁺ and CD8⁺T cells were scarce in the tumor-bearing mice regardless of the agents used for treatment (up to about 5%). On the day 7 of treatment, IFN-γ-producing cells were significantly increased among CD8⁺, but not CD4⁺T cells. Importantly, G3139-LNPs were much more potent in inducing IFN-γ production by CD8⁺T cells (26.84%), compared to G3139 (19.42%) and empty LNPs (10.38%) (FIGS. 18A-18B).

There was no significant change of the INF-γ expression in CD4⁺ cells on either day 2 (3.16%) or day 7 (5.73%) after treatment with G3139-LNPs. These findings show that G3139-LNPs can induce an adaptive immune response that shifts to type 1 with an increase in INF-γ-producing CD8⁺ cytotoxic T cells (CTLs). This was further supported by identification of a large number of CD4⁺ and CD8⁺T cells in the tumors. Since tumor regression was observed in the mice treated with G3139-LNPs started from day 4 to 7 post treatment, the frozen tumor sections from the mice treated with G3139-LNPs, G3139, or LNPs for 7 days were analyzed by immunohistochemistry (IHC) for the infiltrated CD4⁺, CD8⁺ and CD122⁺ cells. As shown in FIGS. 19A-19D, CD4⁺ and CD8⁺ cells were found ubiquitously infiltrating the tumor tissue except for the necrotic areas in tumors from the mice treated with G3139-LNPs, but not those that were treated with G3139 or LNPs (FIGS. 19A-19D).

In addition, more CD122⁺ cells were detected in the tissue sections of tumors from the mice treated with G3139-LNPs than those from the mice treated with G3139 or LNPs, although the number of infiltrating CD122⁺ cells was much lower than those of CD4. and CD8⁺ cells in the same group (FIG. 19D). These results show that adaptive immunity may have played a critical role in rejection of established tumors and that G3139-LNPs can induce a strong adaptive anti-tumor immunity.

### Discussion of Example B

The LNPs encapsulating ODN were produced by an EtOH dilution/diafiltration method. The ODN were efficiently loaded into LNPs by EtOH dilution/diafiltration method, and G3139 was encapsulated into LNPs which dramatically changed its plasma clearance profile and enhanced its immunostimulatory effects.

DC-Chol as the cationic lipid and incorporation of PEG-DSPE into the LNPs aided in providing long circulation time and serum stability. DC-Chol has a titratable tertiary amine group with apparent pKa of 7.8. When the external pH is close to neutral pH, DC-Chol is partially deprotonated resulting in reduced surface charge, as confirmed by zeta potential analysis. PEG on the LNP surface can decrease uptake of particle by the RES, resulting in longer in vivo circulation. In addition, DC-Chol served as a steric barrier that minimizes LNP aggregation and fusion during the formulation synthesis and storage. This LNP formulation has enabled high encapsulation efficiency for the ODN and good colloidal stability.

Western blot results showed that the G3139 had Bcl-2 down-regulatory activity in human KB cells, but not in murine L1210 cells (FIG. 13). Moreover, upon removal of CpG motifs from G3139, the resulting non-CpG containing G4126 formulated in LNPs did not show immune stimulatory effect or antitumor activity (FIG. 15).

G3139-LPNs induced a much stronger cytokine response and a much greater therapeutic activity than free-G3139. The increased activity of the nanoparticles is believed to be due to more efficient uptake of the LNPs by tumor resident macrophages and dendritic cells, resulting in greater local immunoactivation, as shown by immunohistochemical staining of the tumor sections (FIG. 19). Keeping LNP particle size below 200 nm provides important for efficient extravasation of the particles at the site of the tumor and maintaining long systemic circulation time.

Increased uptake of G3139-LNPs by phagocytic cells provides greater accessibility for CpG motifs to TLR-9 than free G3139. G3139-LNPs dramatically promoted proliferation of both DCs and NK cells based on BrdU incorporation (FIGS. 17A-17D). Since murine NK cells express little TLR-9 and thus may not be directly activated by CpG motif, it is possible that G3139-LNPs-stimulated DCs and/or macrophages produce factors that indirectly stimulated NK cell proliferation.

Example B shows that the G3139-LNPs were highly effective therapeutic agents. In fact, 1.5 mg/kg dose was very effective in activating immune responses and inhibit tumor growth in mice. In contrast, both low (1.5 mg/kg) and high (5 mg/kg) dose of free G3139 did not inhibit tumor growth (FIG. 14).

Elevated expression of INF-γ as well as high proliferation of innate effector cells, including NK cells and DCs, play pivotal roles in acquired immunity. The CD8+ cells were apparently up-regulated to express elevated levels of INF-γ at 7 days after treatment. In addition, IHC staining of tumor sections clearly demonstrated much higher levels of CD4+and CD8+ cells infiltrating the tumor and greater tumor cell killing in G3139-LNP group than free G3139 or empty LNP treatment groups had (FIG. 19). These data show that G3139-LNPs induced protective immunity by activating type 1 innate as well as acquired immunity. It should be noted that G3139 has no antitumor effect on its own (FIG. 14, FIG. 15) in the L1210 model, while it did have an effect on spleen expansion (FIG. 16), NK and DCs expansion (FIG. 17) and induction of INF-γ (FIG. 18). This appears to be a contradiction. One explanation is that tumor infiltration of CD8+T cells was more critical for antitumor activity than peripheral cytokine production (FIG. 19). G3139-LPN was shown to be significantly more potent than G3139 in inducing CD8+T cell infiltration in tumors (FIG. 19). This was likely a result of the high tumor accumulation level of LNPs (FIG. 12D).

### Example C

Rituximab (anti-CD20 antibody) represents a major therapeutic advance for B-cell malignancies including chronic lymphocytic leukemia (CLL). Rituximab was conjugated on cationic liposomes carrying bcl-2 targeted antis-sense oligonucleotides (G3139) or Mcl-1 siRNA for CLL delivery. The rituximab directed immunoliposomes (anti-CD20 ILP) have a sub-100 nm particle size and are slightly positive charged. The nanosize structure was confirmed by Atomic force microscopy. In comparison to non-formulated ODN (free ODN), the formulated ODN anti-CD20 ILP shows selectively and preferential targeting of B-CLL Cell. Effective binding and selective uptake of anti-sense ODN is correlated with the CD20 expression levels on the cells.

Anti-CD20 ILP mediated ODN delivery enhances the intracellular Bcl-2 down-regulation both in Raji B malignant cell line and CLL patient cells, which increase cell apoptosis determined by Annexin V/PI staining. The uptake of ODN loaded anti-CD20 ILP was examined by confocal microscopy analysis. FAM labeled ODNs (FAM-ODNs) are partially intracellular distribution in Raji and B-CLL cells. The application of anti-CD20 ILP was extend to siRNA delivery for CLL. The undesirable immunostimulation by G3139 containing CpG dinucleotides can be significantly inhibited when it was encapsulated into anti-CD20 ILP. Expression of co-stimulatory molecules including CD40, CD80, CD86 and HLA-DR can be remarkably reduced, compared to free G3139 treated B-CLL cells.

### Introduction for Example C

CD20 antigen expressed on B-cell malignancies is a well-established B-cell target. The advantages of using such a target exist in that it is a very selective target on CLL cells and the expression level of CD20 is relatively high compared to some other targets. More importantly, high-specific targeting CD20 monoclonal antibodies (mAbs) are commercially available. Rituximab (Rituxan), a chimeric monoclonal antibody against the CD20 cell surface antigen, have been in clinical trials for the treatment of chronic lymphocytic leukemia (CLL). Rituximab affects antitumor activity through complement-mediated cytotoxicity (CDC), and antibody-dependent cell-mediated cytotoxicity (ADCC). The anti-tumor activity of rituximab in CLL can be further increased via the ODNs mediated down-regulation bcl-2 family membrane proteins such as Bcl-2 and Mcl-1. Accordingly, rituximab conjugated lipids-based delivery system hold great promise for efficient delivery of ODNs to CLL. However, since rituximab alone undergoes limited internalization in CLL cells, the main challenge for developing rituximab conjugated nanocarriers is to achieve efficiently intracellular delivery.

Example C presents the use of rituximab conjugated cationic immunoliposomes (Anti-CD20 ILPs) as a safe vehicle for delivering ODNs, achieving high in vitro transfection efficiencies and good targeting specificity to human B-Cell malignancies. The G3139 ODNs were stabilized with a natural cationic polymer-protamine and surrounded by liposomes with a rituximab targeting moiety on the surface. Example C shows whether anti-CD20 ILPs can selectively deliver ODNs to B-cell malignancies and enhance bcl-2 and Mcl-1 down-regulation. This strategy is useful to enhance existing therapeutics for the treatment of CLL disease and other B malignant cell diseases.

### Materials and Methods for Example C

Materials. Egg phosphatidylcholine (egg PC) and methoxy-polyethylene glycol (MW=2000 Da)-distearoyl phosphatidylethanolamine (DSPE-PEG) and were obtained from Lipoid (Newark, N.J.). 3β-[N-(N′,N′-Dimethylaminoethane)-carbamoyl]Cholesterol (DC-Chol) and DSPE-PEG-maleimide (DSPE-PEG-Mal) were purchased from Avanti Polar Lipids, Inc (Alabaster, Ala.). 2-Iminothiolane (Traut's reagent) and other chemicals were purchased from Sigma Chemical Co. (St. Louis, Mo.). G3139 (5′-TCT CCC AGC GTG CGC CAT-3′), G3622 (5′-TAC CGC GTG CGA CCC TCT-3′) [SEQ ID NO:6] and a FAM-terminus modified ODN (5′-(6) FAM-TAC CGC GTG CGA CCC TCT-3′), [SEQ ID NO: 7], were phosphorothioate modified and customer synthesized by Alpha DNA Inc. (Montreal, Calif.).

Rituximab (chimeric anti-CD20 Rituxan, IDEC Pharmaceuticals, San Diego, Calif., and Genentech, Inc., South San Francisco, Calif.) was obtained from RX USA (Jamaica, N.Y.). Trastuzumab (Herceptin) and Campath (anti-CD52) were used. Anti-CD37 was purchase from BD Biosciences (San Diego, Calif.).

Cell lines, B-CLL cells and PBMC cells. Raji and Jurkat leukemia cell lines obtained from American Type Culture Collection (Manassas, Va.), were cultured in RPMI 1640 media supplemented with 10% heat-inactivated fetal bovine serum (FBS, Hyclone Laboratories, Logan, Utah), 2 mM L-glutamine (Invitrogen, Carlsbad, Calif.), and penicillin (100 U/mL)/streptomycin (100 ug/ml; Sigma-Aldrich, St. Louis) at 37° C. in an atmosphere of 5% CO₂. Blood was obtained from CLL patients with informed consent under a protocol approved by the hospital internal review board. Peripheral blood mononuclear cells (PBMCs) were separated from heparinized venous blood of the B-CLL patients and from leukocyte fractions of the healthy donors by density gradient centrifugation using Ficoll-Paque (Pharmacia LKB Biotechnology, Piscataway, N.J.). B-CLL cells were further isolated by using B cell Isolation Kit II (Miltenyi Biotec, Auburn, Calif.). PBMCs and B-CLL cells were incubated in RPMI 1640 media supplemented with 10% fetal bovine serum.

Preparation of Alexa fluor-488 labeled antibodies. Rituximab was fluorescently conjugated by an amine-reactive compound, Alexa fluor 488 5-SDP ester (Invitrogen, Carlsbad, Calif.). Rituximab solution (1.0 mg/ml) was exchanged to sodium bicarbonate buffer by dialysis with Slide-A-Lyzer Dialysis Unite (Rockford, Ill.) against 0.1 M sodium bicarbonate solution at for 1-2 hr. Then 1.2 μl of Alexa fluor 488 5-SDP ester in DMSO solution of 10 mg/ml was added into rituximab in (NaHCO₃, pH=8.3) buffer for 1 hr at room temperature. The resultant solution was put into Slide-A-Lyzer dialysis tube and dialyzed against PBS (pH=7.4) overnight. The resultant Rituximab-Alexa 488 was collected and diluted to certain concentration, sterilized via 200 nM polymer membrane filter and was stored in 4° C. Herceptin-Alexa 488 was synthesized as the same procedures.

Preparation of Rituximab directed cationic immunoliposomes. An ethanol dilution method was modified to prepare the ODN encapsulated liposomal nanoparticles. Briefly, protamine sulfate in citrate acid (20 mM, pH4) was mixed with lipids (DC-Chol:Egg-PC:PEG-DSPE (molar ratio)=28.0:70.0:2.0) at mass ratio of lipids:protamine=12.5:0.3, followed by addition of oligonucleotide in citrate acid (20 mM, pH4) at oligonucleotide:lipids:protamine (weight ratio)=1:12.5:0.3. The complexes were then dialyzed against citrate acid (20 mM, pH4) for 1 hours and then further dialyzed against HBS buffer (145 mM NaCl, 20 mM HEPES pH7.4) overnight, using a DispoDialyzer (Spectrum Labs, Rancho Dominguez, Calif.) with a Molecular Weight Cut-Off of 10,000 dalton. A post-insertion method was adopted to incorporate antibody ligands into preformed liposomes carrying ODNs. Rituximab (anti-CD20) was reacted with 10× Traut's reagent (2 hr, Room temperature) to yield sulfhydryl modified antibodies. The anti-CD20-SH was then reacted to micelles of Mal-PEG-DSPE at a molar ratio of 1:10, and then incubated with ODN loaded liposomes for 1 h at 37° C. Targeted liposomes with anti-CD20 to total lipid ratios of 1:2000 (0.05 mol %) were prepared. Herceptin-targeted control liposomes or anti-CD37 ILPs were prepared by coupling trastuzumab (Herceptin) or anti-CD37 instead of anti CD20 to the liposomes using the same method. For binding study, the post-inserted immunoliposomes carrying FAM-ODN were further separated to remove free PEG conjugated antibodies by CL-4B column.

Characterization of liposomal nanoparticles. The particle sizes of LPs were analyzed on a NICOMP Particle Sizer Model 370 (Particle Sizing Systems, Santa Barbara, Calif.). The volume-weighted Gaussian distribution analysis was used to determine the mean vesicle diameter and the standard deviation. The zeta potential (4) was determined on a ZetaPALS (Brookhaven Instruments Corp., Worcestershire, N.Y.). All measurements were carried out in triplicates. The ODN content in targeted and non-targeted liposomes were determined by electrophoresis in 15% polyacrylamide gel with EtBr staining. The structures of the LPs and anti-CD20 ILPs were investigated by atomic-force microscopy (AFM). A Digital Instruments (Santa Barbara, Calif.) Nanoscope III atomic force microscopy (AFM) was used to image Morphology of performed ODN loaded cationic liposomes (LP) or anti-CD20 ILP. Images were recorded in both height and amplitude modes. Colloidal stability of the ILPs in plasma were determined by incubating the ILPs with 50% human plasma for varying amount of time at 37° C., followed by measuring particle size at various time-points.

Cell surface immunostaining. Cells (0.5×10⁵/ml) were incubated at with PE-labeled anti-CD20, mouse IgGᵢ isotype control antibodies (BD Biosciences, San Diego, Calif.) or Rituximab-Alexa 488, Herceptin-Alexa 488 at 4° C. for 30 minutes. The cells were then spun down at 300 g for 10 minutes and rinsed twice with cold phosphate-buffered saline (PBS, pH=7.4) and analyzed by FACS) for 30 minutes at 4° C. CD20 surface expression levels were analyzed by FACS on a Beckman Coulter EPICS XL (Beckman Coulter). Ten thousand events were collected under list mode.

Immunofluorescence assays of co-stimulatory molecules. At the time points indicated, cells were washed in ice-cold phosphate-buffered saline (PBS) and were stained for surface antigens. Monoclonal antibodies (mAb) against CD40 (5C3), CD80 (L307.4), CD86 (IT2.2), and HLA-DR and appropriate isotype controls were purchased from BD Biosciences (San Diego, Calif.).

Binding study. For the binding study, cells were pre-incubated with 1 uM free FAM-ODN or 1 uM FAM-ODN encapsulated LP, anti-CD20 ILPs and Herceptin ILPs for 60 minutes at 37° C. The incubation and wash procedure was identical to the surface staining protocol. For cell lines, cells were split the night before and fresh cells were used for immunostaining as described for B-CLL cells.

Specificity study. Mixed Raji and Jurkat cells (1:1) were co-cultured for 4 hr ahead. The mixed cells or PBMC cells were pre-incubated with 0.5 uM free FAM-ODN or 0.5 uM FAM-ODN encapsulated anti-CD20 ILPs for 60 minutes at 37° C. The cells were then spun down at 300 g for 10 minutes and rinsed twice with cold PBS (pH=7.4) for FACS analysis.

Laser-scanning confocal microscopy. Binding and uptake of the liposomes in Raji and CLL cells were examined by laser scanning confocal microscopy. Cells were incubated with LP, Her ILP and anti-CD20 ILP liposomes for 4 hrs at 37° C. and washed twice with phosphate-buffered saline (PBS) followed by fixation with 2% paraformaldehyde (PFA) for 30 minutes. Nucleus was stained with 1 ug/ml of DRAQ5™ (Biostatus Limited, Leicestershire, United Kingdom) for 5 minutes at RT. These cells were mounted on a poly-D-lysine coated cover glass slide (Sigma-Aldrich, St. Louis, Mo.). Green fluorescence of FAM-DON and blue fluorescence of DRAQ5 were analyzed, and merged images were produced by using Multi-photon Imaging Systems and LSM Image software.

Evaluation of apoptosis and cell viability by flow cytometry. The apoptosis of cells was measured using Annexin V-FITC/propidium iodide (PI) staining followed by FACS analysis according to manufacture's protocol (BD Pharmingen). Unstained cell sample, and cells stained with Annexin V-FITC or PI only were also processed for compensation. Results were presented as % cytotoxicity, which was defined as (% Annexin V+ and/or PI+ cells of treatment group)−(% Annexin V+ and/or PI+ cells of cells of media control). FACS analysis was performed using a Beckman-Coulter EPICS XL cytometer (Beckman-Coulter, Miami, Fla.). Ten thousand events were collected for each sample and data was acquired in list mode. System II software package (Beckman-Coulter) was used to analyze the data.

Assessment of Bcl-2 down-regulation by Western-blot. The Western blot was carried out to evaluate the Bcl-2 protein level. After the delivery of G3139 and scrambled ODN loaded liposomes, the cells were incubated with a lysis buffer containing a protease inhibitor cocktail (CalBiochem, San Diego, Calif.) on ice for 20 min. The pellets were removed after centrifugation the lysate at 13,000 rpm at 4° C. for 10 min at. The supernatant was collected and the protein concentrations were determined by BCA assay (Pierce, Rockford, Ill.). After the separation of proteins in a 12% SDS-polyacrylamide gel, the proteins transferred to a PVDF membrane and unspecific binding of Bcl-2 to it antibodies was blocked with 5% milk in PBS-buffered saline containing 0.1% Tween-20 (PBST) for 80 mins. The membranes were then incubated with primary anti-human Bcl-2 at 4° C. overnight, followed by incubation with horseradish peroxidase-conjugated goat antimouse IgG. Membrane was then developed with Pierce SuperSignal West Pico or Dura Extended Duration Substrate (Pierce) and imaged with Kodak X-OMAT film (Kodak, Rochester, N.Y.). To normalize the protein loading amount in SDS-PAGE, the membrane was washed by PBST and blotted by polyclonal goat anti-human beta-actin antibody (Santa Cruz, Santa Cruz, Calif.) and secondary antibody rabbit anti-goat IgG (Pierce).

Statistical analysis. Analysis was performed by statisticians in the Center for Biostatistics, the Ohio State University, using SAS software (SAS Institute Inc. Cary, N.C., USA). Comparisons were made using a two-sided α=0.05 level of significance. Mixed effects models were used to account for the dependencies in the cell donor experiments, and analysis of variance (ANOVA) was used for the cell line experiments. Synergy hypotheses were tested using interaction contrasts.

### Results for Example C

### Free G3139 does not Significantly Down-Regulate bcl-2 Expression in Raji Cell and Primary B-CLL Cells in the Absence of Cationic Liposomes

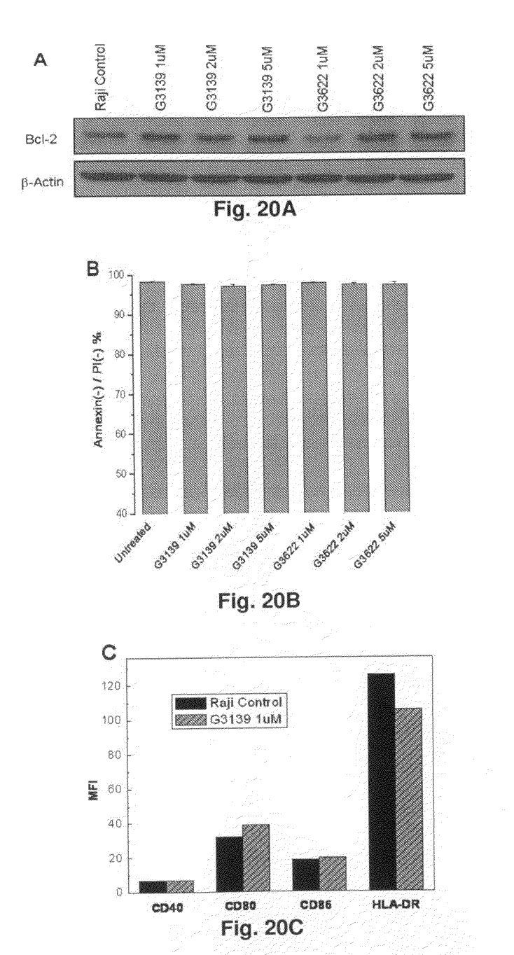

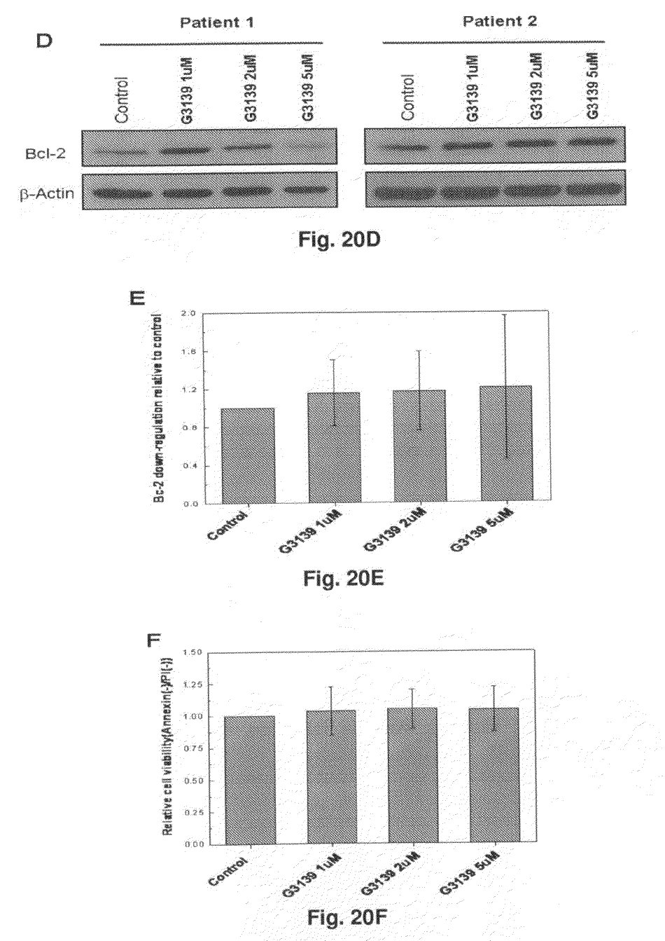

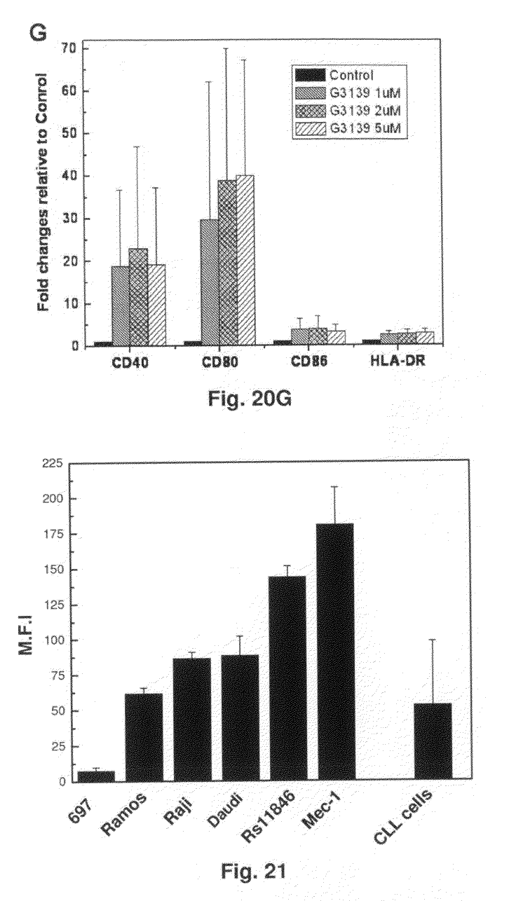

As shown in FIG. 20A, no marked difference of bl-2 protein level was observed between free G3139 and G3622 treated cells in comparison to medium control cell. Cell viability study by Annexin V/PI staining also showed no noticeable apoptosis after treatment by free G3139 (FIG. 20B).

Since G3139 containing unmethylated CpG dinucleotides may active B cells and lead to expression of co-stimulatory molecules, expressions of typical surface markers (CD40, CD80, CD86 and HLA-DR) were assessed for immunostimulation by flow cytometry. After treatment by G3139 for 481r, Raji cell didn't show much difference on levels of surface marker expression (FIG. 20C), which means no activation. FIG. 20D shows the two representative western blot results out of n=10 CLL patient B cells. At high concentration of 5 μM, CLL patient 1 showed the significant bcl-2 down-regulation but no remarkable difference of bcl-2 protein level was found in CLL patient 2, compared to medium control. Statistically, as presented in FIG. 20E, the average protein level of bcl-2 protein level was up-regulated under the treatment of G3139 at various concentrations (1 μM, 2 μM and 5 μM). Cell viability study by Annexin V/PI staining (FIG. 20F) and measurement of co-stimulatory molecules expression (FIG. 20G) confirmed the proliferation and activation of CLL B cells, respectively. Particularly, the expressions of CD40 and CD80 were significantly up-regulated after treatment by free G3139. Overall, without cationic liposomes, no antisense-mediated inhibition of blc-2 synthesis was achieved with G3139. Instead, the CpG motifs of G3139 remarkably induced expression of co-stimulatory molecules as well as Bcl-2 levels of primary B-CLL cells.

Rituximab is a Good Antibody for Targeting to B Cell Lines and Primary B-CLL Cells.

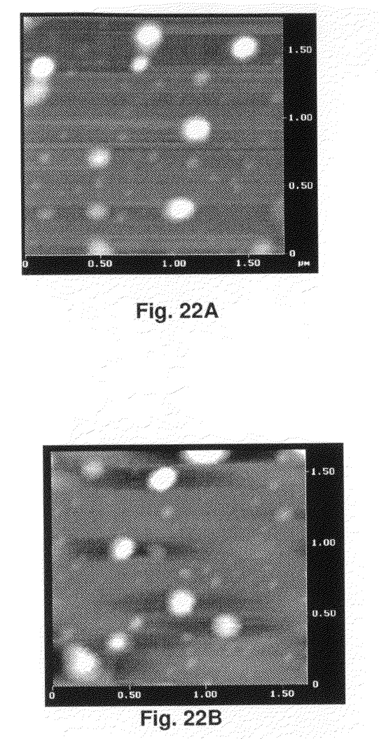

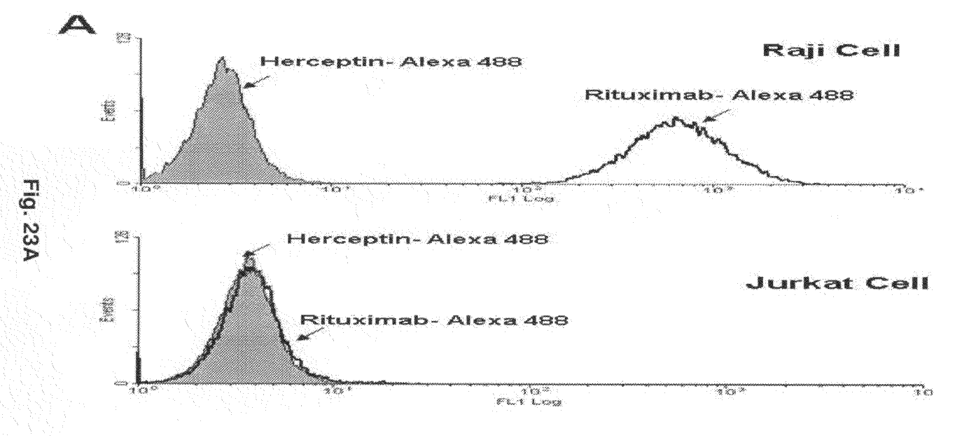

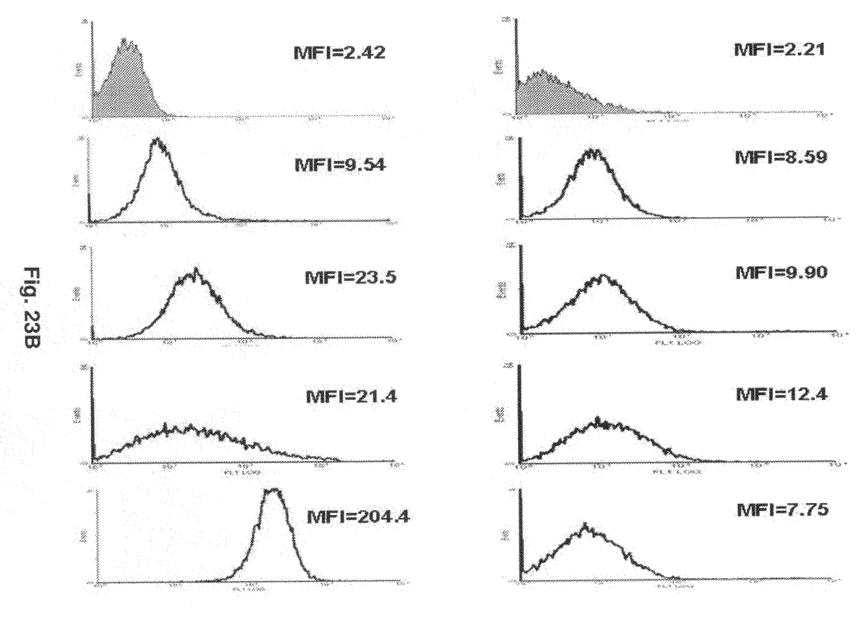

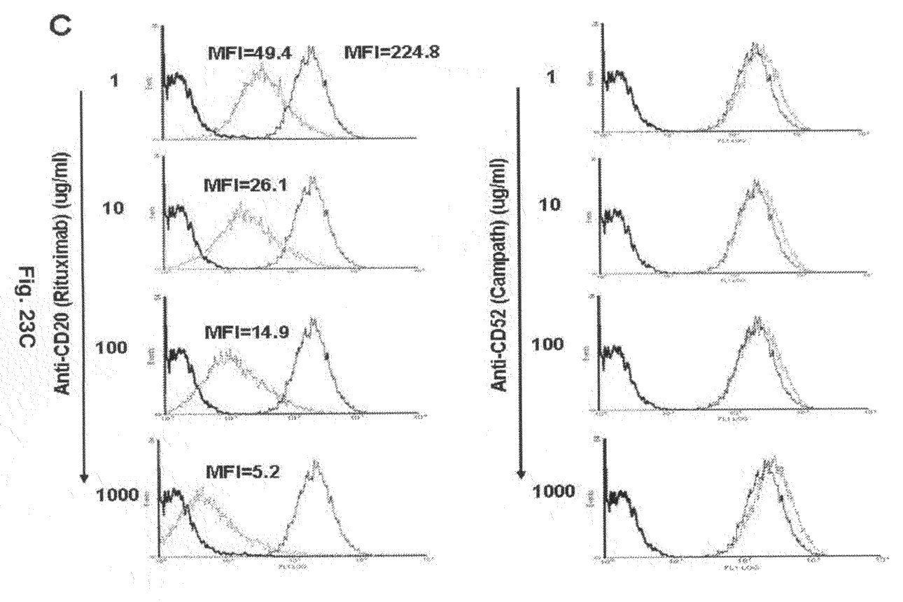

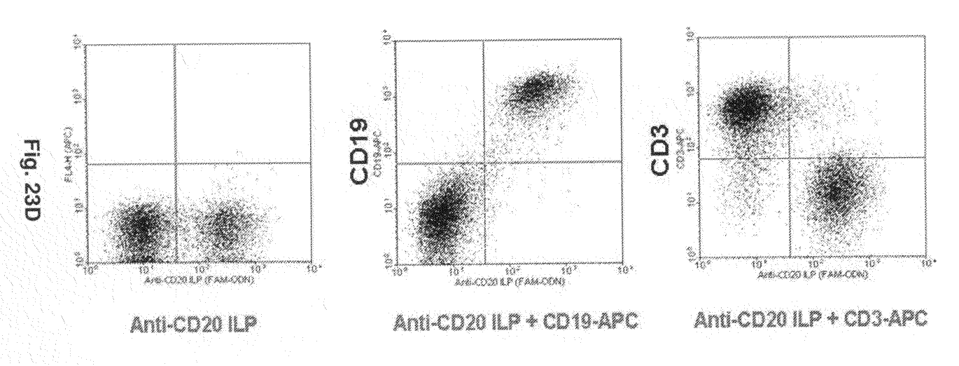

Rituximab is a chimeric monoclonal antibody directed at CD20, which is an established B-cell target. To examine the exact expression of CD20 directed by rituximab, rituximab antibody was first fluorescently conjugated with Alexa Fluor-488. Assessment of CD20 receptor expression was determined by cytometric analysis after immunostaining six major B cell lines and B-CLL cells using rituximab-Alexa 488 (FIG. 21). It was observed that CD20 receptors are highly expressed on the tested B cell lines except 697 cell line. In particular, the expressions of CD20 directed by rituximab are extremely high on RS11846 and Mec-1 cells. As seen in FIG. 21, all B-CLL cells samples express CD20 but the intensities are variable. On average, high expression of rituximab against CD20 was observed on B-CLL cells, which is comparable with the expression on Raji and Ramos cells. We then selected the Raji cell line for further experiments. This result shows that it is possible to target to B cell lines and B-CLL cell using rituximab as a targeting molecule.

Preparation and Characterization of Rituximab (Anti-CD20 Antibody) Conjugated Cationic Immunoliposomes (Anti-CD20 ILPs).

In Example C, cationic liposomes (LPs) were used to achieve high stability and high encapsulation efficiency. The ethanol dilution method was applied to make LPs. The cationic lipid of DC-Chol was chosen for encapsulating the electrostatic self-assembled protamine/ODN complexes. Rituximab and herceptin control were incorporated onto the formed ODN-LPs by post-insertion of the rituximab or herceptin conjugated with PEG-DSPE. As characterized in Table 6, all of the ODN loaded LPs have approximately the same average diameter of 50-70 nm and are slightly positive charged (+2˜0.6 mV).

TABLE 6
Characterization of various LP formulations^{a}

| Formulation | Particle size (nm)^{b} | Zeta potential (mV) |
|---|---|---|
| Naked LP | 49.3 ± 5.2 | 4.22 ± 0.82 |
| Herceptin conjugated LP | 55.6 ± 7.4 | 4.25 ± 0.65 |
| (Her ILP) | | |
| Rituximab conjugated NLP | 56.3 ± 7.5 | 2.09 ± 0.31 |
| (Anti-CD20 ILP) | | |

^{a}LP:DC-Chol/EggPC/DSPE-PEG = 28/70/2 (molar ratio); lipids/ODN//Protamine = 12.5/1/0.3 (weight ratio); 0.05 mol % Herceptin or Rituximab was conjugated on LP. The encapsulation efficiency of ODN was above 90%.
^{b}The representative data is from the mean of three separate measurements.

The particle size of antibody coated LPs are slightly bigger than that of naked LPs. Atomic force microscopy (AFM) imaging was used to further determine morphologies of ODN-encapsulated LPs and anti-CD20 LPs. As shown in FIG. 22, both ODN-LPs and ODN-anti-CD20 ILPs demonstrated spherical nano-structures although significant difference has not been found between them. The colloidal stability of ODN-loaded LPs was evaluated by monitoring changes in the mean diameter of the LPs. No significant changes in particle size were observed during several weeks.

Anti-CD20 ILP mediated delivery is CD20 antigen-specific and anti-CD20 ILP selectively binds to B malignant Raji cells in mixed populations with Jurkat cells.

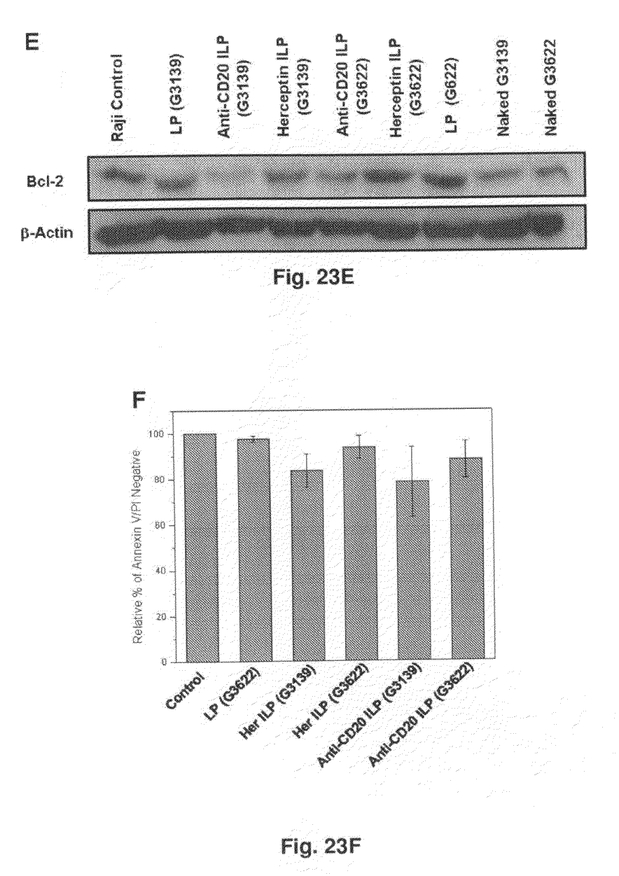

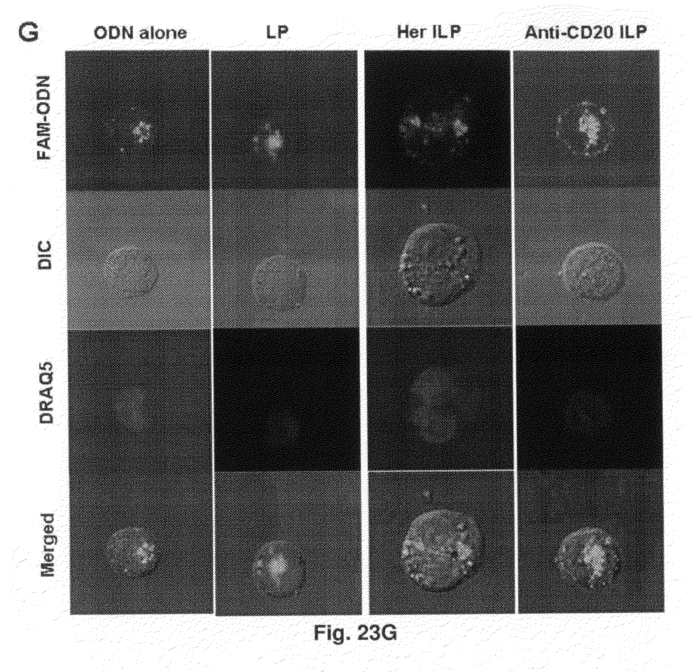

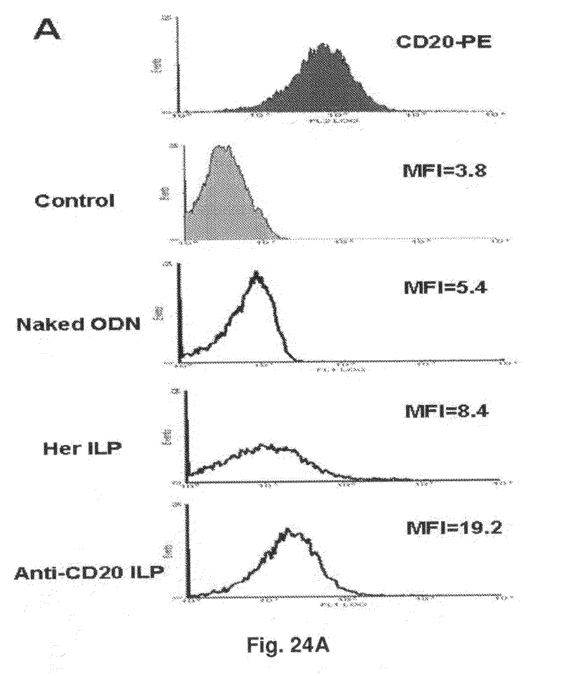

The expression of rituximab against CD20 receptor on Raji (B malignant cell line) and Jurkart (T malignant cell line) cells was assessed by direct immunostaining of cells with rituximab-Alexa 488 (FIG. 23A).

Herceptin-Alexa 488 was used as negative antibody control for immunostaining. According to FIG. 23A, it is reasonable to choose Raji cell and Jurkat cell as B (CD20+) and T (CD20−) model cell line, respectively. Fluorescently labeled ODN with G3139 mismatch sequence (FAM-ODN) were used for the binding study. Raji and Jurkat cells were incubated with free FAM-ODN or various LP formulated FAM-ODN at 37° C. for 1 hr and green fluorescence was determined by flow cytometry. As shown in FIG. 23B, the enhanced binding of anti-CD20 ILP carrying ODN was observed in Raji cells that over-express CD20 antigen. Jurkat cells (CD20−) showed low binding efficiency, which is comparable with the intensities of LP or Her ILP treated cells. In contrast, Her ILP mediated ODN delivery did not show marked difference between Raji and Jurkat cells. This finding shows that anti-CD20 ILP mediated delivery is CD20 antigen specific. Moreover, FIG. 23B showed some non-specific interactions of free FAM-ODN to Raji and Jurkat cells. That might be from the ODN strong bound to serum proteins, which facilitates the uptake of free ODN by cells via endocytosis. However, compared to cationic liposomes (either LP or Her ILP) mediated delivery, the binding intensity of free ODN is much lower than those of cationic liposomes formulated ODNs.

A competitive blocking study, in which Raji cells were pre-incubated with extra Rituximab (anti-CD20) or Campath (anti-CD52) from low to high concentrations, showed that Rituximab was able to almost completely block the anti-CD20 mediated binding whereas CD52 antibody had no any blocking effect (FIG. 23C). This result strongly supports the CD20 binding specificity of rituximab directed cationic liposomes.

To demonstrate the selectivity of anti-CD20 ILP, the mixed Raji (B cell line) and Jurkat (T cell line) populations were treated by FAM-ODN loaded anti-CD20 ILP and analyzed by flow cytometry. As seen in FIG. 23D, green fluorescently labeled ODNs were preferentially delivered to Raji cells that were identified by the second staining of APC labeled CD19. Thus, FAM-ODN incorporated anti-CD20 ILPs can selectively bind to Raji cells but almost no Jurkat cells.

Anti-CD20 ILP Carrying G3139 Enhances bcl-2 Down-Regulation and Induces Apoptosis in Cultured Raji Model Cell Line.

The antisense Bcl-2 effect of G3139 in various formulations was evaluated at protein levels on Raji after 48 hr treatment (FIG. 23E). All transfection experiments were performed in 10% serum containing RPMI1640 medium. Raji cells treated by anti-CD20 ILP formulated G3139 showed the best bcl-2 down-regulation compared to other conditions. In contrast, no obvious bcl-2 down-regulation was observed following treatment with G3622 (reverse sequence), indicating that the observed antisense effect was sequence specific. The LP treatment of Raji cells also demonstrated a higher silencing effect than G3139 on its own. Induction of apoptosis by free ODN and various formulated ODNs was further evaluated by Annexin V/PI staining (FIG. 23F). The significant increase of apoptosis in anti-CD20 ILP was observed. We next used confocal microscopy to investigate the ability of various cationic liposomal formulations to bind and deliver FAM-ODN to Raji cell (FIG. 23G). Free FAM-ODN treated Raji cell was used control. After 24-hour exposure of Raji cell to the fluorescently labeled ODN at various conditions, FAM-ODNs (green) in LP and Her ILP as well as free ODN alone were intracellularly distributed in Raji cell, whereas FAM-ODN in anti-CD20 ILP showed partially intracellular distribution and some nanoparticles still attached on the cell membrane. These results demonstrated that anti-CD20 ILP can be partially internalized by Raji cells, although rituximab is a non-internalizing antibody on its own. The partial internalization might be caused by cationic nature of the resultant anti-CD20 ILP (shown in Table 6).

Specific Delivery of Anti-CD20 ILP is Correlated with CD20 Expression Level on Primary B-CLL Cells and ODN Loaded Anti-CD20 ILP but not Free ODN Shows B Cell Selectivity in PBMC Cells.

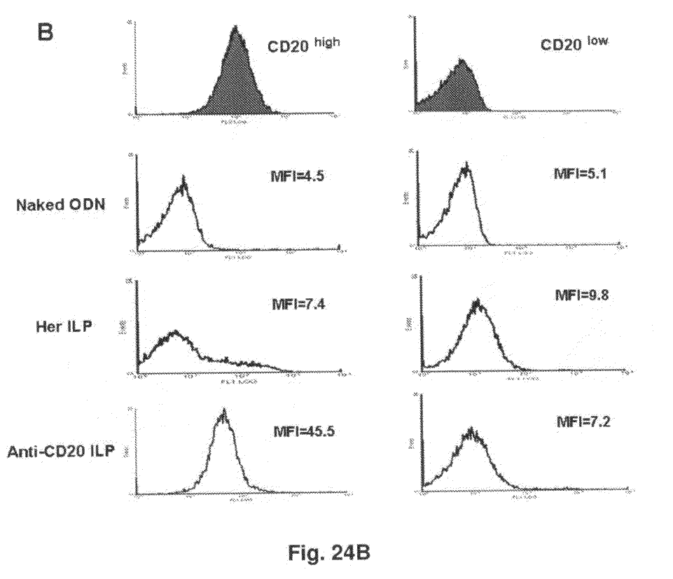

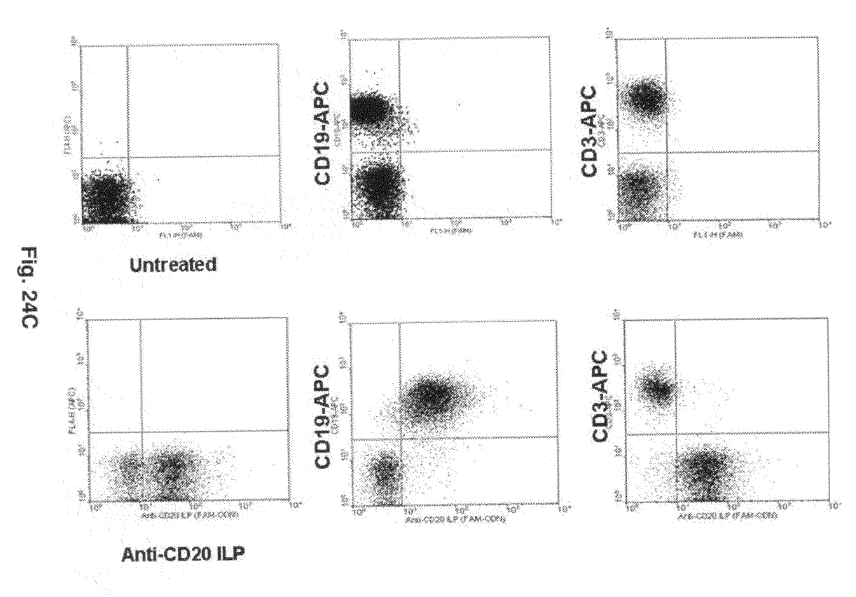

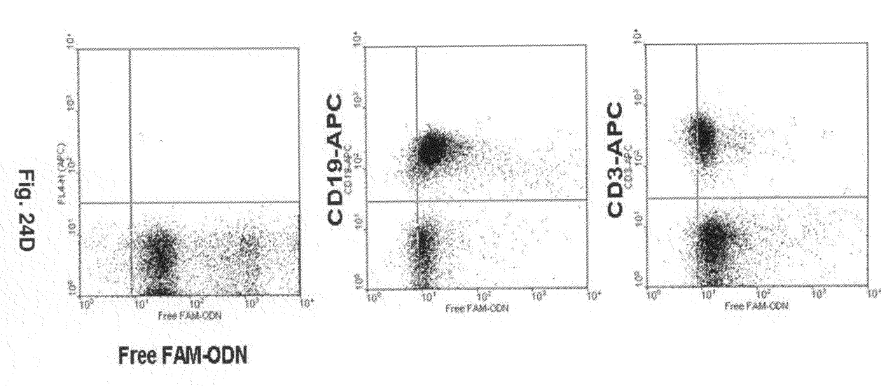

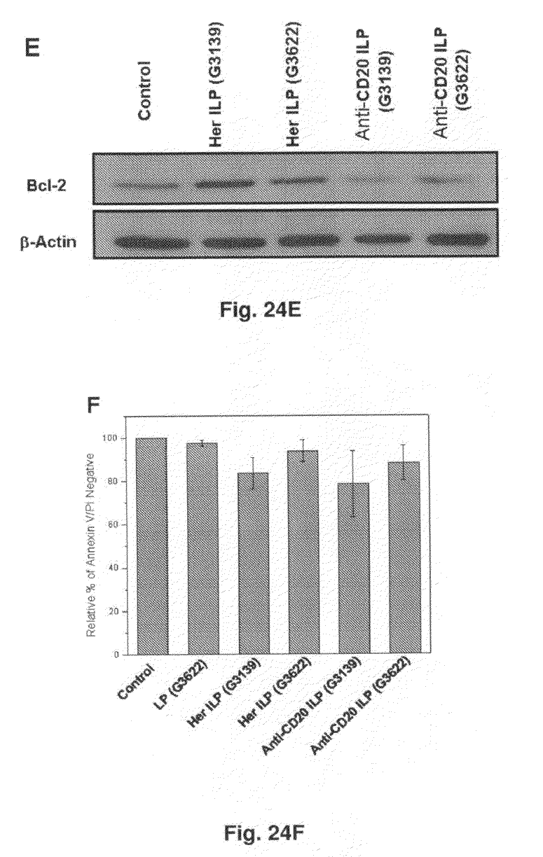

The CD20 antigen specific targeting of rituximab directed cationic liposome was further examined in primary B-CLL cells. FIG. 24A presented as a representative binding study of free FAM-ODN and various LP formulated FAM-ODN on primary B-CLL cells. The CD20 expression level (the top histogram of FIG. 24A) of this CLL patient is on average of all tested CLL cells and its corresponding targeting capacity was evaluated as histogram. Anti-CD20 showed the enhanced binding efficiency when compared to Free ODN and Her ILP treated cells. However, the mean fluorescence intensity was relatively low. Using similar comparison study, we tested a few B CLL cells with a variety of CD20 expression. Two extreme B CLL examples were illustrated in FIG. 24B.

Rituximab directed cationic immunoliposomes showed CD20 antigen specific in B-CLL cells as well. The more CD20 expression, the more strong CD20 specific binding (left panel, FIG. 24B). The binding capacity of anti-CD20 ILP is significantly dependent on the CD20 expression on CLL cell surfaces. For CD20 negative CLL cells, anti-CD20 did not show obvious CD20 binding. Indeed, slight binding was detected, comparable with the non-specific binding intensity of Her ILP (right panel, FIG. 24B). Similar with the mixed population of Raji (B cell line) and Jurkat (T cell line) cells, the selectivity of anti-CD20 mediated delivery was confirmed in peripheral blood mononuclear cells (PBMCs) isolated from patients with CLL (FIG. 24C).

FAM-ODNs were preferentially delivered to B cells in PBMC that were recognized by the second staining of APC labeled CD19. FAM-ODN incorporated anti-CD20 ILPs bind selectively to B cells but not T cells, which were consistent with the specificity study in Raji and Jurkatt mixed cells (FIG. 23D). In contrast, free FAM-ODNs (non-formulated) unselectively bind to both B and T cells (FIG. 24D) in the same PBMC cells used in FIG. 24C. Western blot analysis of bcl-2 protein was performed following exposure to Her ILP or anti-CD20 ILP formulated G3139 and G3622 at 2 uM for 48 hr in B-CLL cells (FIG. 24E). Again, anti-CD20 ILP formulated G3139 showed enhanced bcl-2 down-regulation when compared to other treatments. Relative percentage of B-CLL cell viability normalized to medium control was carried out to examine the induced apoptosis by various treatments. The percentage of viable cells was determined by Annexin V/PI staining and was analyzed by flow cytometry. As seen in FIG. 24F, the increased apoptosis in G3139 loaded anti-CD20 ILP was observed. The rituximab directed G3622 ILPs also showed the induced apoptosis, which was probably from cross-linked killing of anti-CD20 ILPs.

The Innate CpG Immunostimulation of G3139 can be Significantly Inhibited when Encapsulated into Anti-CD20 ILPs.

Due to CpG motifs in G3139 sequence, free G3139 has shown B-cell activation, accompanying with significant up-regulation of surface markers such as CD40, CD80, CD86 and HLA-DR (FIG. 20G). To examine the effect of G3139 in anti-CD20 ILP on immunostimulation of B-CLL cells, anti-CD37 directed cationic immunoliposomes were used as positive control. CD37 has been proved as a good B target and anti-CD37 has faster internalization rate. As shown in FIG. 25A, the innate CpG immunostimulation of G3139 was significantly inhibited in both anti-CD20 and anti-CD37 formulations. The inhibition would avoid the undesirable CpG effect and achieve real anti-sense bcl-2 down-regulation. To further confirm the inhibiting ability of CpG immunostimulation by anti-CD20 ILP, another phosphothiated CpG ODN(ODN 2006) was selected. Similar with G3139, free ODN 2006 showed significantly up-regulate costimulatory molecules (CD40, CD80, CD86 and HLA-DR) but anti-CD20 formulated ODN remarkably inhibited the B-cell activation, characterizing with no significant up-regulation of expression of costimulatory molecules.

### Discussion of Example C

Rituximab and bcl-2 anti-sense ODN by rituximab directed cationic immunoliposomes (anti-CD20 ILP) encapsulating G3139 provide B cell-type specific targeting with enhanced cell entrance. The enhanced B cell-type delivery is demonstrated herein both in malignant cell lines and primary B-CLL cells. Moreover, a similar strategy is also useful for the Mcl-1 siRNA delivery for CLL.

Treatments for CLL with anti-sense or RNA interference (RNAi) represent new therapeutic strategies. G3139 is an 18-mer phosphorothioate ODN targeting for bcl-2 down-regulation. Inhibition of bcl-2 expression by G3139 might render bcl-2 overexpressing malignant B cells more susceptible to chemotherapy in CLL.

In general, cationic vectors such as lipofectin and lipofectamine are required to provide sufficient uptake of anti-sense ODNs into cells in vitro. Free G3139 did not show obvious down-regulate bcl-2 expression in Raji cell in the absence of cationic lipid nanoparticles (FIG. 20A). Although two out of 10 tested CLL patients give responses, the average bcl-2 level expression at three different concentrations did not decrease (FIGS. 20D, 20E). On the contrary, innate CpG motifs in G3139 significantly increases co-stimulatory molecules including CD40, CD80, CD86 and HLA-DR similar to that observed with B-cell activation (FIG. 20G). This undesirable immunostimulation effect might render the slight bcl-2 up-regulation in primary B-CLL cells (FIG. 20F), which is consistent with the reported results by intracellular flow bcl-2 staining.

Due to polyanionic properties and large molecular weight, ODNs lack cell-type specific targeting and low cellular membrane permeability. Although some naked antisense ODNs are able to bind to certain components in serum, following uptake by cells, the intracellular amount of ODN uptake is limited. Furthermore, free anti-sense ODN can lead to nonspecific knockdown and toxic side effects. These concerns were confirmed in our specificity study of free ODN. FAM labeled ODN can non-specifically get into both B and T cells (FIG. 24D), which might cause global repression of anti-apoptotic proteins and result in some unpredictable immunoresponses.

Example C provides a novel strategy for achieving CLL targeted delivery using ligands that selectively bind to B cell surface but not T cell. CD20 represents a unique antigen restricted to cells of B lineage and almost all of the B cell malignancies express CD20 (FIG. 21). Rituximab directed at CD20 antigen has been widely used as an immunotherapeutic agent in CLL clinic treatment. Thus Example C provides an immunolipid nanoparticle design for B-cell type targeted delivery that can be based on rituximab. Although CD20 is, in general, not internalizing, it can become an internalizing antibody in some special cases. In addition, anti-CD20 directed immunolipid nanoparticle still can enhance the drug therapeutic efficiency if fast-releasing drug like vincristine (VCR) was loaded into anti-CD20 immunolipid nanoparticles (anti-CD20 ILP) and it showed the comparable improved therapeutic effects over VCR loaded anti-CD19 ILP. Anti-CD20 ILP increases chances of drug releasing into cells by enhanced binding to B malignant cells although the whole liposomal particles are not uptaken by cells.

In Example C, cationic lipid nanoparticles were chosen to obtain high loading efficiency of anti-sense ODN. Cationic lipid nanoparticle can penetrate the cell membrane, thus facilitating gene/ODN delivery. Thus, rituximab coated cationic immunolipid nanoparticle was designed to enhance binding to B cells, followed by increasing uptake because of its positive-negative electrostatic interaction with cell membranes.

To prepare rituximab and herceptin coated immunolipid nanoparticles, the “post-insertion” method was adopted. The incorporation of rituximab and herceptin on LPs slightly increased the particle size. The particle size of all resultant LPs is sub-100 nm and particle surfaces are positively charged (Table 6). The nanosize structure of LP and anti-CD20 ILP was confirmed by Atomic force microscopy analysis (FIG. 22).

Rituximab conjugated cationic immunolipid nanoparticles show the characteristic of CD20 antigen specific targeting both in Raji model cell line and primary B-CLL cells isolated from patients (FIG. 23B, FIG. 24A). In Raji cells, anti-CD20 ILP significantly increase the fluorescence intensity of FAM-ODN, which is ˜10 folder stronger than FAM-ODN loaded LP and Her ILP and ˜20 folder stronger than that of free FAM-ODN. The enhanced binding efficiency of FAM-anti-CD20 ILP is closely dependent on the CD20 level expressions on B-CLL cells (FIG. 24B). To minimize the non-specific binding of cationic lipid nanoparticle on its own, lower cationic lipid (DC-Chol) was used. However, it still gives some fluorescence intensity in binding study of Raji cell and B-CLL cells. It also accounts for the no 100% blocking achievement even if very high extra rixumab (1000 ug/ml) was used (FIG. 23C). The B-cell type selectivity of anti-CD20 ILP was confirmed in both mixed cell populations of Raji and Jurkat as well as PBMC cells (FIG. 23D, FIG. 24C), which realizes our initial design. The enhancement of bcl-2 down-regulation by G3139-anti-CD20 ILP was found in Raji cell (FIG. 23E).

The increased fold of bcl-2 down-regulation is not as significant as that was obtained in flow data (FIG. 23B). As seen in FIG. 23G, the partial uptake of ODN in anti-CD20 ILP by Raji cells might be a possible reason. The enhanced bcl-2 down-regulation is also reflected on the increased apoptosis in FIG. 23 F. Furthermore, we found that the average binding intensity of FAM-ODN-anti-CD20 ILP in Raji cell is much lower than that in CLL B cells, which is correlated with the relatively low CD20 expression on B-CLL cells in comparison to Raji cell (FIG. 24A). Consequently, the improved down-regulation of bcl-2 in B-CLL cells is not as potent as observed in Raji cell (FIG. 24E). As shown in FIG. 23 F and FIG. 24 F, the LP alone is not toxic. Rituximab directed immunolipid nanoparticles carrying G3622 induced some apoptosis that might be caused from the cross-linking of rituximab by lipid nanoparticles, thus showing that rituximab directed cationic lipid nanoparticles are effective nanocarriers for B-CLL targeted delivery.

Avoiding the undesirable immunoeffects and taking full advantages of desired gene or protein silencing is essential for the clinical application of these therapeutic agents. Unfortunately, most anti-sense ODNs and siRNAs contain immunostimulatory motifs. Due to the CpG dinucleotide in G3139, it causes significant immunostimulation characteristics of up-regulation of co-stimulatory molecules and bcl-2 protein. The immunolipid nanoparticles such as CD20 ILP and CD37 ILP can inhibit the activation of G3139. Some surface markers like CD86 and HLA-DR can achieve completely inhibition. This finding was further confirmed in study of ODN 2006, a classic CpG ODN (FIG. 25). One explanation is that CpG encapsulated into immunolipid nanoparticle may bypass the recognition by TLR 9 in B cells.

The rituximab (CD20 antibody) directed cationic immunolipid nanoparticles illustrated B-cell-type selectivity both in B malignant cell lines and CLL cells in vitro. The anti-CD20 ILP can inhibit the CpG immunostimulation of G3139 and take full advantage of its blc-2 antisense design. The improved bcl-2 and Mcl-1 down-regulation were achieved in anti-CD20 ILP. The Example C also provides a strategy for improving the existing antisense clinic trial and RNA interference therapeutics in CLL.

### Example D

Example D provides a targeted delivery of Ones to malignancy B cells by using antibody directed liposomal immuno-nanoparticles (INP), including delivering G3139, an As-ODN against Bcl-2, via Rituximab (anti-CD20) conjugated INP.

Example D also provides a delivery system for Mcl-1 siRNAs, based on novel anti-CD37 mAb conjugated INP (anti-CD37 INP). Additionally, Example D provides incorporating another antibody such as anti-CD20 or anti-CD19 into anti-CD37 INP to further improve efficiency and specificity of Mcl-1 siRNAs. A combination of anti-CD37 and other antibodies provide highly specific targeting function to individual patient cells. Example D provides, not only development of a novel clinical agent for CLL therapy, but also, technological advances in nanoparticle design and synthesis with broad applications in oligonucleotide therapeutics.

Chronic Lymphocytic Leukemia (CLL).

CLL represents the most common type of adult leukemia and is incurable with standard therapy. In the CLL, chemotherapeutic agents such as fludarabine and chlorambucil have been effective in a subset of patients. However, non-specific effects and even non-response of these drugs obstruct their therapeutic efficacy in the clinic.

In addition to the rituximab, alemtuzumab that targets CD52, an antigen expressed on normal lymphocytes as well as many T- and B-cell neoplasms has been used for first-line treatment for CLL. But the major drawback of alemutuzumab is the damage in T cells of CLL patients.

Bcl-2 or Mcl-1 as a Therapeutic Target in CLL and Other B-Cell Malignancies.

The anti-apoptotic proteins such as Bcl-2 and are important members of the Bcl-2 family that plays critical roles in promoting the survival of lymphocytes and hematopoietic stem cells. Mcl-1 and Bcl-2 preserve the mitochondrial integrity by binding to mitochondrial porin channels, thus inhibiting mitochondrial destabilization and subsequent initiation of apoptosis. Multiple studies have demonstrated that the anti-apoptotic subset (Bcl-2, Bcl-xl, and Mcl-1) is linked to drug resistance and poor treatment outcome in a variety of tumor types.

Down-regulation of Bcl-2 or Mcl-1 by siRNA or antisense molecules is sufficient to initiate apoptosis in some cell lines, while in other cell types, down-regulation of Mcl-1 is insufficient to initiate apoptosis but promotes sensitivity to chemotherapy and radiation. Thus, down-regulation of Mcl-1 or Bcl-2 plays a primary role in the initiation of apoptosis in B-cell leukemia, which provides justification for the development of Bcl-2 or MeI-1-targeted therapies.

Use of Oligonucleotides as Therapeutic Reagents.

Oligonucleotides, including antisense oligonucleotides (As-ODNs) and small interfering RNA (siRNA) are emerging as promising therapeutic agents against a variety of diseases such as cancer and leukemia. AS-ODNs are ˜20 nt in lengths and act by targeting specific mRNAs through heteroduplex formation inside the cell, thereby inducing RNase H activation, translational arrest, or by altering splicing. In vitro activity of AS-ODNs requires delivery via invasive methods, such as electroporation and complexation to a transfection agent. However, clinical trials on AS-ODNs invariably have used free ODNs. Vitravene (formiversen), a phosphorothioate AS-ODN for treatment of CMV retinitis in AIDS patients, was the first ODN to gain approval by the U.S. FDA. Formiversen is somewhat unique in that it is given by direct injection into the vitreous body of the eye. For systemic administration, in order to counter rapid clearance due to renal excretion, the ODNs in clinical trials have been given via prolonged continuous intravenous infusion. Despite these measures, the clinical efficacy of AS-ODNs has been limited in most cases and the expected target down regulation is often not observed. For example, in a clinical trial on an AS-ODN G3139 targeting Bcl-2, a significant fraction of the patients showed up-regulation of Bcl-2, rather than the intended target down regulation.

siRNA is much more efficient for gene silencing both in vitro and in vivo, comparing to AS-ODNs. RNAi takes full advantage of the physiological gene silencing machinery, which can efficiently mediate the cleavage of targeted mRNA molecules. siRNAs consist of duplexes of oligoribonucleotides that are 19- to 23-nt each in length, containing a sense-strand and an antisense strand. siRNAs interact with Argonaute-2 (Ago-2) to form RNA-induced silencing complexes (RISCs), which degrades the sense-strand of the siRNA and then cleaves target mRNAs that are perfectly complementary to the antisense strand. siRNAs also exhibit significant miRNA effect against targets that are not perfectly complementary. This results in off-target effects of siRNA. siRNAs are much more potent in inducing target gene silencing on a per molar basis compared to AS-ODNs. siRNA mediated down-regulation of Mcl-1 can be used to mediate caspase independent apoptosis in acute lymphocytic leukemia cell lines, primary CLL B cells and lymphoma cell lines. In combination with standard chemotherapy, siRNA therapy can also reduce chemo-resistance, suggesting the potential use of siRNA therapy for treating many malignant diseases. However, ODNs therapeutic remains particularly challenging, due to difficulties in transduction of lymphocytes and other primary blood cells. In addition, as siRNAs are often disseminated throughout the body, targeted systemic delivery approaches are warranted. Low transfection efficiency, poor tissue penetration, and nonspecific action on bystander cells and immune activation by siRNAs have posed limitations on the therapeutic application in vivo.

Challenges for ON Delivery.

As polyanionic macromolecules, ODNs face multiple obstacles in reaching their intracellular site of action, thus present a significant problem for drug delivery. In fact, there is no natural mechanism for these highly hydrophilic macromolecules to traverse the cellular membrane and bioavailability of these agents on their own is minuscule. Nevertheless, the delivery of ODNs is somewhat less challenging than delivery of therapeutic genes, which has thus far been the limiting factor for the successful clinical application of gene therapy. This is because ODNs, which are typically less than 30 nt or bp, are significantly smaller in size than therapeutic genes (>7 kb). In addition, ODNs are produced by chemical synthesis, which allows for purity of the materials and introduction of chemical modifications that provides greater metabolic stability or that enables synthesis of derivatives with greater bioavailability.

In particular, for delivery to solid tumor, there are four major barriers for ODNs to gain access to malignant cells and take effect on the intracellular targets. First, the ODNs must avoid rapid degradation by serum nucleases, rapid excretion by renal filtration and/or clearance by the reticuloendothelial system (RES). Second, the ODNs must gain access to the target cells by crossing the capillary endothelium and travel in the extracellular matrix. Third, the ODNs must be taken up by the target cells, typically through an endocytotic process. Finally, the ODNs must be released from the endosomes and reach intracellular targets, such as loading onto dicer/Ago-2 in the case of siRNA. An effective delivery strategy must take into account the need to overcome all of these barriers, as well as avoid introducing tissue toxicity and undesirable immunoactivation.

Choice of Antibody for Targeted Delivery of siRNA or As-ODNs.

To address the delivery issues of ODNs including poor intracellular uptake, limited blood stability, and non-specific immune stimulation, targeted delivery based on cell type-specific ligands such as monoclonal antibodies has been increasingly recognized as a promising strategy for in vivo application of ODNs. Antibody-based therapeutics has been attractive in cancer and leukemia treatment, because of their high specificity and affinity to target antigens. Therapeutic antibodies such as trastuzumab (Herceptin®), rituximab (Rituxan®) and alemtuzumab (Campath®) have been routinely used in the clinical treatment of breast cancer and leukemia.

Compared to intact antibodies, small antibody fragments, such as scFv and Fab, are less bulky and lack a Fc domain, which may interfere with in vivo delivery. Therefore, antibodies or antibody fragments represent an interesting class of molecules for enhancing the delivery of therapeutic reagents to target tumor cells. However, problems including the potential for immunogenicity and the high cost should be taken into account in application of antibody-mediated delivery.

ILNs containing anti-CD20 antibody are useful to efficiently deliver the FAM-ODN into primary CLL B cells and B cell lines selectively. This delivery is further enhanced using pharmacological agents such as lenalidomide (which causes internalization of the CD20 antigen). Since single antigen expression on cell surfaces varies from patient to patient, it is a good strategy to combine these antibodies together to achieve the maximal binding and delivery efficiency for individual patient.

### Results for Example D

Targeted delivery of Mcl-1 siRNAs using CD37-ILN mediates down-regulation of Mcl-1 protein levels and promotes increased spontaneous apoptosis in CLL B cells.

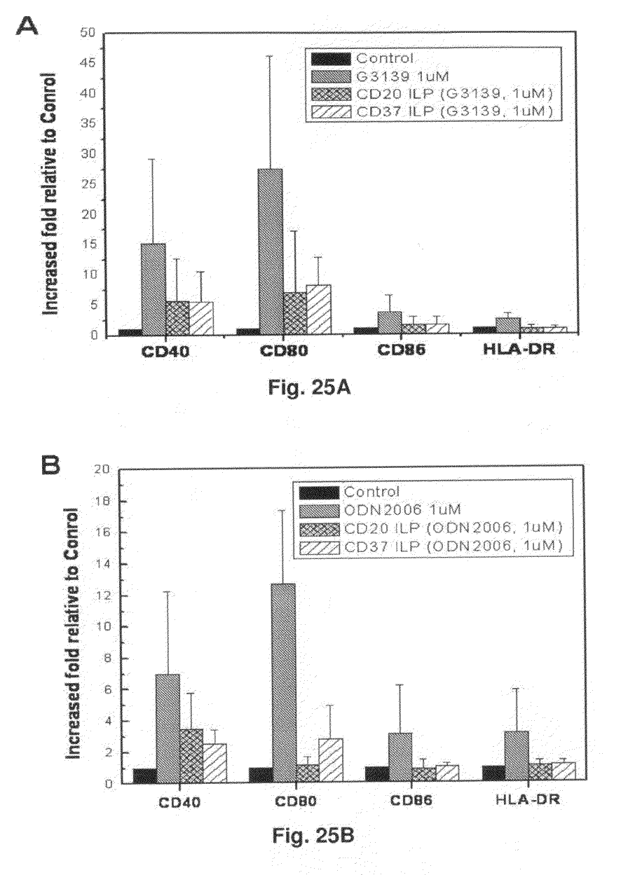

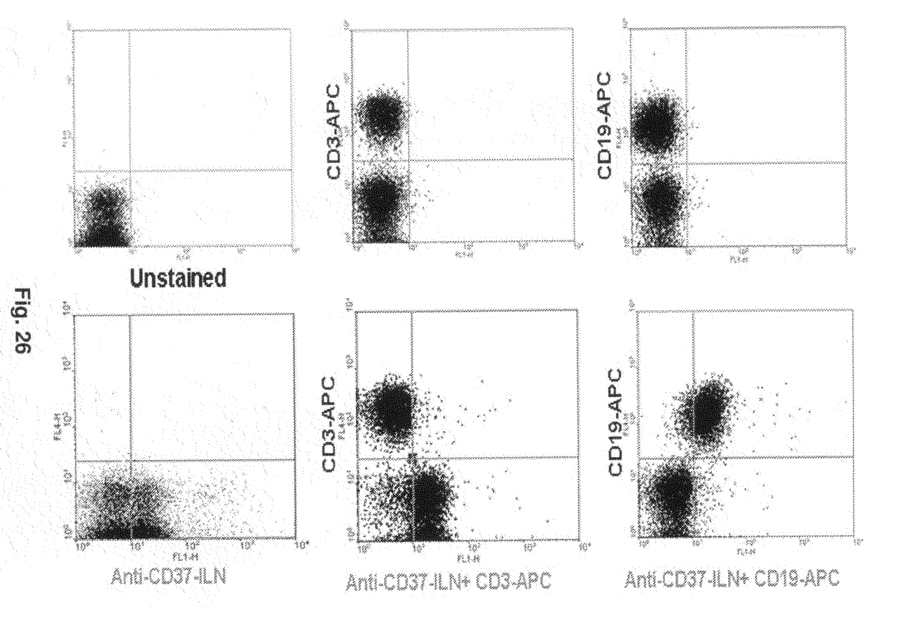

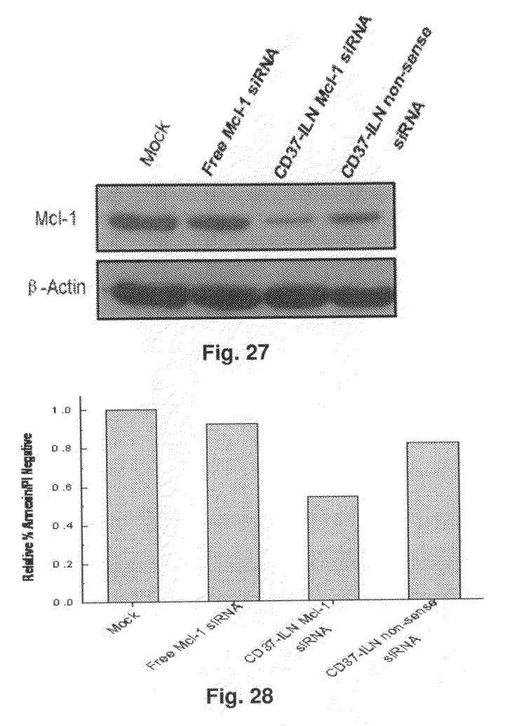

Anti-CD37 ILN containing FAM-ODN was used for determining the cell type specific binding. Binding to CD19+B cells but not to CD3+T cells in the peripheral blood mononuclear cells from CLL patients is shown in FIG. 26. In order to determine if Mcl-1 down-regulation will alter the spontaneous apoptosis in CLL B cell, CD19⁺ CLL B cells were treated with media (mock), CD37-ILN with Mcl-1-specific siRNA or nonsense siRNAs control. Cells transfected with the WM-specific siRNA containingCD37-ILN exhibited significant decrease in Mcl-1 protein (FIG. 27) and decreased viability as detected by Annexin V/PI staining compared to the nonsense siRNAs controls by 24 hrs.

Dual Antibody Mediated Delivery Via Immuno-Liposomal Nanoparticles (ILNs).

Single antibodies and combined antibodies were incorporated onto ILNs by the post-insertion method. The antibodies were chemically modified with PEG-DSPE, followed by mixing with FAM-ODN loaded lipid nanoparticles. The binding efficiency of immunolipid nanoparticles onto Raji cells were analyzed by conventional flow cytometry. As seen in FIG. 28, the lipid nanoparticles coated with combined antibodies (CD20/CD37) show much higher green fluorescence intensity, compared to anti-CD20 INP or anti-CD37 INP. The combinational design of using dual antibodies can be further for siRNA delivery to B cell leukemia.

### Discussion of Example D

Oligonucleotides targeted towards anti-apoptotic protein Bcl-2 or Mcl-1 provide a novel approach for overcoming resistance to biological and chemotherapeutic agents. These results demonstrate that down-regulation of Bcl-2 or Mcl-1 enhanced the apoptosis in Raji model cell line and B-CLL cells. It has been also shown that, when given as free ODN, only very low level of cytoplasmic ODN concentration was achievable, while no cytoplasm-to-nucleus drug trafficking and target down-regulation were observed⁷². Commercial transfection agents, such as NeoPhectin™ and Lipofectamine™ rely on electrostatic mechanism for cellular uptake. Unfortunately, these agents cannot be used in vivo because they lack selectivity for leukemia cells, are cytotoxic and do not function properly in the plasma environment. Therefore, in order to improve the efficacy and tumor specificity of Mcl-1 siRNA therapy and provide a paradigm for in vivo delivery of siRNAs to down-regulate anti-apoptotic proteins in B cell malignancies in general and CLL in particular, new delivery strategies are needed.

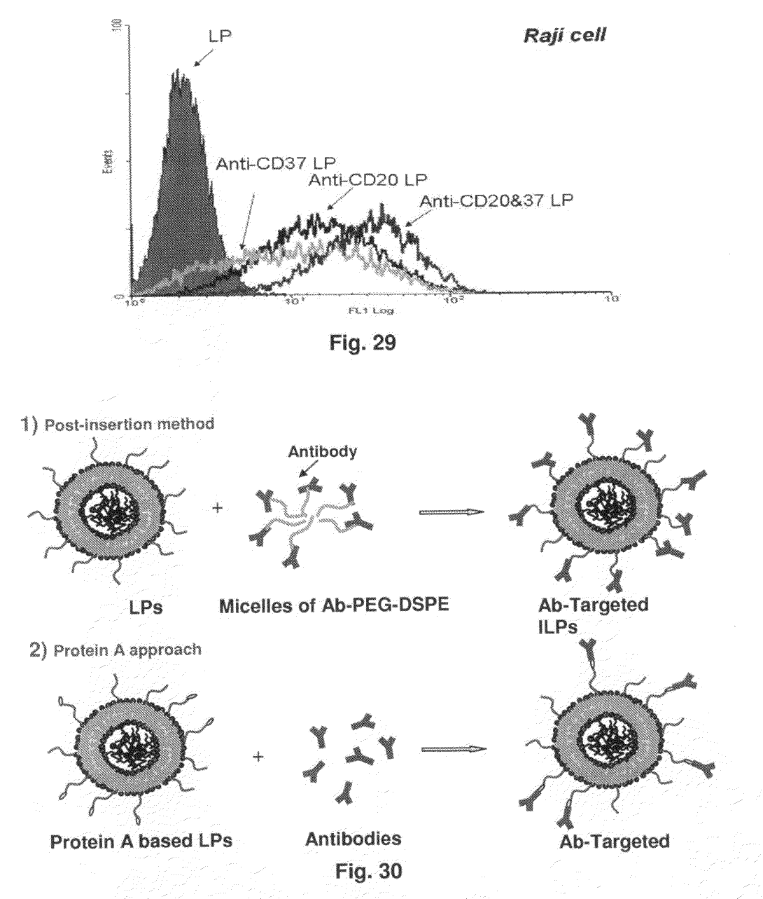

Due to relatively high expressions of CD20 and CD37 antigens on B-CLL cells, rituximab and CD37 antibody were used as targeting molecules for delivering ODNs. Using anti-CD37 INP of siRNA as an example, the basic rationale and principle for using INP-mediated As-ODN and siRNA delivery is shown in FIG. 29. Anti-CD37 based INPs are designed to target CD37, which represents an internalizing CLL cellular antigen that is known to mediate endocytosis of anti-CD37 mAb. In addition to specific targeting of CD37+ CLL cells, the INP formulation is designed to provide stability to siRNA against plasma nucleases, prolonged systemic circulation time, and efficient endosomal release of the siRNA and down-regulation of the Mcl-1 target. The INPs are taken up by leukemia cells via binding to CD37, followed by endocytosis and endosomal release of the siRNA drug.

The strategy described herein is useful to form compounds that modulate the critical Mcl-1 protein which has been shown to render resistance to apoptosis. This strategy is also useful for making therapeutic approaches for B cell leukemia. In addition, the novel strategy described herein is useful to advance the technologies of nanoparticle synthesis and oligonucleotide therapeutic delivery.

Non-limiting examples of uses of such strategies include:

i) CD20-ILN formulations for targeted delivery of G3139 to B-CLL cells having increased sensitivity of B-CLL cells to fludarabine after Bc-2 down-regulation;

ii) CD37-ILN formulations for targeted delivery of Mcl-1 siRNA to B-CLL cells having increased sensitivity of B-CLL cells to fludarabine and/or Rituximab after Mcl-1 down-regulation;

iii) CD37-ILN formulations in combination with one or more antibodies for dual- or multi-Ab targeted delivery of Mcl-1 siRNA to B-CLL cells;

iv) RIT-INP formulation where the formulation of anti-CD37 INP is altered ofr modulated sensitivies;

v) dual targeting strategies based on Anti-CD37; and

vi) INP formulations having enhanced binding and/or down-regulation efficacy.

For example, a schematic illustration of a Protein A based immunolipid nanoparticles for formulating dual or multi Ab targeted delivery is shown in FIG. 30.

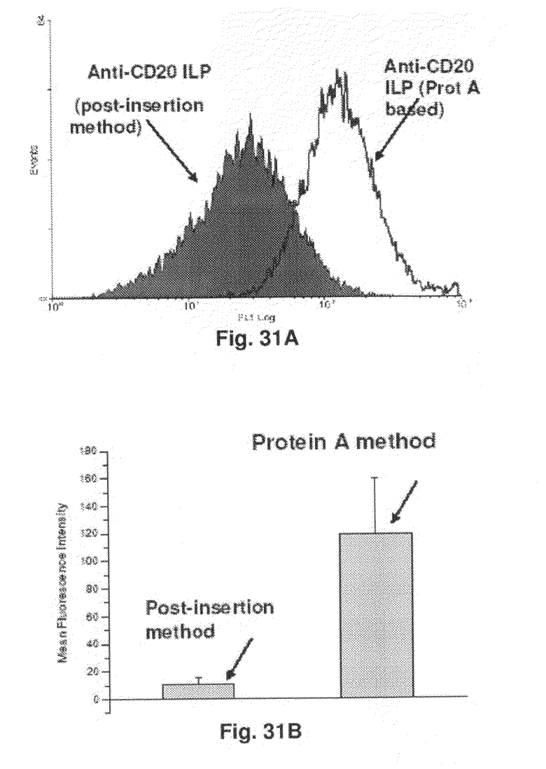

FIGS. 31A-31B show a comparison of binding efficiency of Anti-CD ILPs prepared by two approaches: Post-insertion approach, and Protein A approach.

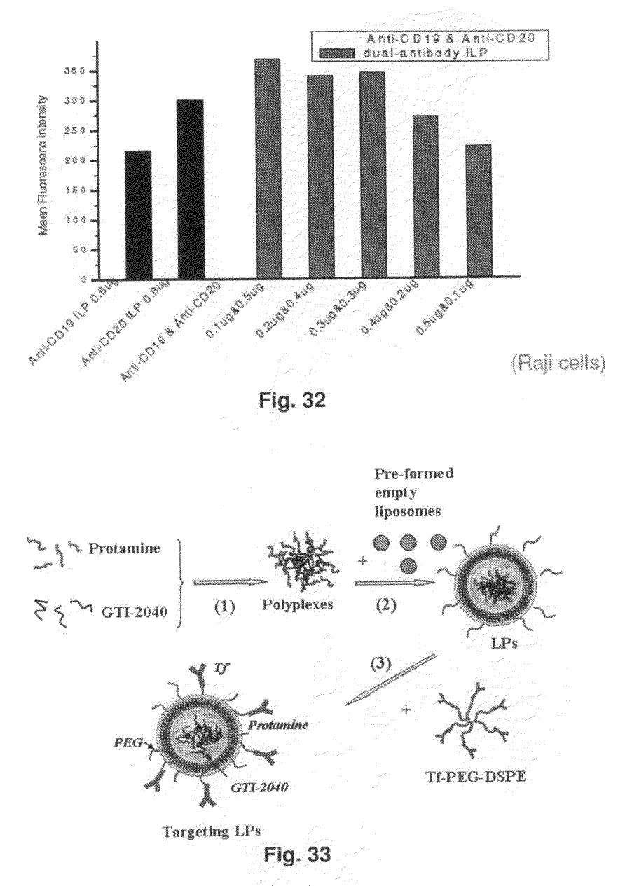

FIG. 32: Graph showing enhanced binding efficiency by dual-AB ILPs of Raji cells. FIG. 32 shows the enhanced binding efficiency by dual-Ab ILPs. comparing Anti-CD19 ILP at 0.6 μg, and Anti-CD 20 ILP at 0.6 μg, to the Dual-Ab ILPs Anti-CD19+Anti-CD 20 at differing concentrations of: 0.1 μg+0.5 μg; 0.2 μg+0.4 μg; 0.3 μg+0.3 μg; 0.4 μg+0.2 μg; and 0.5 μg+0.1 μg.

It is to be noted that similar results were achieved with Dual-Ab ILPs of Anti-CD19+Anti-CD 37 ILPs; and Anti-CD20+Anti-CD 37 in B-CLL cells (data not shown).

### Example E

GTI-2040, an antisense oligodeoxyribonucleotide (ODN) against the R2 subunit of ribonucleotide reductase, is a promising agent for overcoming chemoresistance in acute myeloid leukemia (AML).

Example E shows that the strategy described herein also enhances the clinical efficacy of GTI-2040, where formulations capable of promoting targeted delivery of ODNs into AML cells are used.

In Example E, transferrin (Tf) conjugated pH-sensitive lipopolyplex nanoparticles (LPs) were developed. These nanoparticles can release ODNs at acidic endosomal pH and facilitate the cytoplasmic delivery of ODNs after endocytosis. In addition, Tf-mediated targeted delivery of GTI-2040 was achieved. R2 downregulation at both mRNA and protein levels was improved by 8-fold in Kasumi-1 cells and 2-20 fold in AML patient cells treated with GTI-2040-Tf-LPs, compared to free GTI-2040 treatment. Moreover, Tf-LPs were more effective than non-targeted LPs, with 10-100% improvement at various concentrations in Kasumi-1 cells and an average of 45% improvement at 3 μM concentration in AML patient primary cells. Treatment with 1 μM GTI-2040-Tf-LPs sensitized AML cells to the chemotherapy agent cytarabine, by decreasing its IC₅₀ value from 47.69 nM to 9.05 nM. LPs had an average particle size around 110 nm and a moderately positive zeta potential at ˜10 mV. The ODN encapsulation efficiency of LPs was >90%. The LP structure was studied by Cryo-TEM, indicating several coexisting structures. This study suggests that the combination of pH sensitive LP formulation and Tf mediated targeting is a promising strategy for antisense ODN delivery in leukemia therapy.

### Introduction for Example E

In Example E, we synthesized transferrin (Tf)-conjugated PEGylated lipopolyplex nanoparticles (Tf-LPs) that incorporate protamine as a DNA condensing agent, pH-sensitive fusogenic lipids to improve cytoplasmic delivery, and Tf as the targeting ligand. We show that R2 downregulation at both mRNA and protein levels was significantly improved in AML cells treated with GTI-2040-Tf-LPs, compared to free GTI-2040 treatment.

### Materials and Methods for Example E

Materials. Dioleoyl phosphatidylethanolamine (DOPE) and distearoyl phosphatidylethanolamine-N-[maleimide-polyethylene glycol, M.W. 2000] (Mal-PEG₂₀₀₀-DSPE) were purchased from Avanti Polar Lipids (Alabaster, Ala.). Methoxy-PEG₂₀₀₀-DSPE was purchased from Genzyme Corporation (Cambridge, Mass.). Human holo-Tf, 2-iminothiolane (Traut's reagent), protamine sulfate, cholesteryl hemisuccinate (CHEMS), and other chemicals and reagents were purchased from Sigma Chemical Co. (St. Louis, Mo.). All tissue culture media and supplies were purchased from Invitrogen (Carlsbad, Calif.). All ODNs used in this study were fully phosphorothioated. GTI-2040 (sequence 5′-GGCTAAATCGCTCCACCAAG-3′) [SEQ ID NO: 8] was generously supplied by Lorus Therapeutics Inc. (Toronto, Ontario, Canada). ODN with scrambled sequence (5′-ACGCACTCAGCTAGTGACAC-3′) [SEQ ID NO: 9] and carboxyfluorescein (FAM)-labeled GTI-2040 were purchased from Alpha DNA (Montreal, Quebec, Canada).

Cell lines, patient samples and cell culture. Kasumi-1 and K562 cells were obtained from ATCC (Manassas, Va.). Cells were grown in RPMI medium supplemented with 10% (K₅₆₂) or 15% (Kasumi-1) fetal bovine serum at 37° C. Pre-treatment unselected bone marrow blasts from AML patients were obtained from The Ohio State University (OSU) Leukemia Tissue Bank. Each of the patients signed an informed consent to storing and using his/her leukemia tissue for discovery studies according to institutional guidelines from OSU. Fresh AML primary bone marrow samples were fractionated by Ficoll-Hypaque (Nygaard) gradient centrifugation and grown in RPMI 1640 media supplemented with 15% of human serum and GM-CSF plus Cytokine Cocktail (R&D Systems, MN) at 37° C.

Preparation of Tf-LPs. As shown in FIG. 33, an ethanol dilution method was used to prepare lipopolyplex nanoparticles (LPs) containing GTI-2040, scrambled ODNs or FAM-GTI-2040. Briefly, GTI-2040 ODNs was mixed with protamine in water at a 1:5 molar ratio for 30 minute to form polyplexes. Meanwhile, a lipid mixture of DOPE/CHEMS/PEG-DSPE at a 58:40:2 molar ratio was dissolved in ethanol and then injected into 10 mM HEPES buffer, pH 8.0, to form empty liposomes in 10% ethanol. Then, pre-formed empty liposomes were mixed with the ODN/protamine suspension at a 12.5:1 lipids:ODN weight ratio, followed by vortexing and sonicating to spontaneously form LPs in buffer solution. The final ethanol concentration in the cell culture was less than 1%. A post-insertion method was adopted to incorporate Tf ligand into ODN-loaded LPs (12-15).

Cryogenic transmission electron microscopy (Cryo-TEM). Cryo-TEM imaging was performed as previously described (16). Briefly, samples were examined in a Philips CM120 microscope (Eindhoven, The Netherlands) at 120 kV, using an Oxford CT-3500 cooling holder and transfer station (Abingdon, England). Specimens were equilibrated in the microscope below −178° C., then examined in the low-dose imaging mode to minimize electron beam radiation damage, and recorded at a nominal underfocus of 1-2 μm to enhance phase contrast. Images were acquired digitally by a Gatan MultiScan 791 cooled charge-coupled device camera (Pleasanton, Calif.) using the Digital Micrograph 3.1 software package. Cryo-TEM study was performed at Technion-Israel Institute of Technology, Haifa, Israel.

Characterization of LPs and evaluation of ODN encapsulation efficiency. The particle size of LPs was analyzed on a NICOMP Particle Sizer Model 370 (Particle Sizing Systems, Santa Barbara, Calif.). The volume-weighted Gaussian distribution analysis was used to determine the mean vesicle diameter. The zeta potential was determined on a ZetaPALS (Brookhaven Instruments Corp., Worcestershire, N.Y.). All measurements were carried out in triplicates. The concentration of encapsulated ODN was determined by lysing LPs using 0.5% SDS and 1% Triton X-100, followed by agarose gel electrophoresis to separate SDS, Triton, and ODNs. The density of each ODN band after ethidium bromide staining was measured, and the amount of ODN was estimated by comparing to a series of ODN standards. Encapsulation efficiency was calculated based on the ratio of ODNs in LPs versus the initial amount of ODNs applied.

Study of Tf receptors (TfR) expression. The expression levels of TfR (also known as CD71) on the surface of AML cells were evaluated by surface staining with PE-labeled anti-TfR (anti-CD71) monoclonal antibody (BD Biosciences, San Jose, Calif.) followed by flow cytometry analysis as previously described (13).

Transfection studies. Kasumi-1 and K562 cells were seeded at 5×10⁵/mL density 24 hr before transfection, while patient primary cells were seeded at 3×10⁶/mL density right after they were separated from patient bone marrow. During the transfection, cells were exposed to LPs, Tf-LPs or free ODNs at a final concentration of 1 μM or 3 μM at 37° C. in a CO₂ incubator. In Mock, cells were treated with 10 mM HEPES buffer. After 48 hr, cells were collected and analyzed for R2 mRNA level by real-time qRT-PCR and for R2 protein level by western blot.

Laser-scanning confocal microscopy. Binding and internalization of FAM-GTI-2040-Tf-LPs in AML cells were examined by laser scanning confocal microscopy. Cells were incubated with FAM-GTI-2040-Tf-LPs for 0 hr. and 4 hr respectively at 37° C. and washed twice with PBS followed by fixation with 2% para-formaldehyde for 30 minutes. Nuclei were stained with 20 μM of DRAQ5™ (Biostatus Limited, Leicestershire, United Kingdom) for 5 minutes at room temperature. The cells were mounted on a poly-D-lysine coated cover glass slide (Sigma-Aldrich, St. Louis, Mo.). Green fluorescence of FAM-GTI-2040 and blue fluorescence of DRAQ5 were analyzed, and merged images were produced by using Zeiss 510 META Laser Scanning Confocal Imaging Systems and LSM Image software (Carl Zeiss Microlmaging, Inc., NY, USA).

Quantitative RT-PCR (qRT-PCR). The R2 mRNA level in leukemia cells was evaluated using qRT-PCR as previously described (17). Primer sequences for R2 and ABL, and qRT-PCR conditions are reported in Supplementary section.

Western blot analysis. The R2 protein expression was measured by western blot as previously described (18). Anti-R2 and anti-GAPDH antibodies were purchased from Santa Cruz Biotechnology (Santa Cruz, Calif.) (9). Equivalent gel loading was confirmed by probing with antibodies against GAPDH.

Cell survival studies by MTS assay. Kasumi-1 cells were treated with HEPES buffer (as Mock), GTI-2040-Tf-LP, free GTI-2040 or Scrambled-Tf-LP at 1 μM concentration for 4 hr and then incubated with various concentration of Ara-C (0.0001-10 μM) for 48 hr. Cell survival was then determined by the MTS (3-(4,5-dimethylthiazol-2-yl)-5-(3-carboxymethoxyphenyl)-2-(4-sulfopheyl)-2H-tetrazolium), which is reduced by cells into a formazan product that is soluble in tissue culture medium. Briefly, 20 μL of MTS/PMS (phenazine methosulfate) (ratio 20:1) mixture was added into each well and then incubated for 1-4 hr at 37° C. Absorbance was read at 490 nm on a microplate reader Germini XS (Molecular devices, CA). Three replicates were used at each drug concentration. Data were plotted and IC50 values were calculated using WinNonLin software (version 4.0, Pharsight, Mountain View, Calif.).

Statistical analysis. Data were represented as mean±standard deviations and analyzed by 2-tailed Student's t-test using MiniTAB Program (Minitab Inc., State College, Pa.). p<0.05 was considered statistically significant.

### Results for Example E

Preparation and characterization of LP and Tf-LP nanoparticles. FIG. 33 shows the schematic illustration of the method used for the synthesis of Tf-LPs. Three steps were involved in the process: (1) Negatively charged GTI-2040 ODN was assembled in a complex with positively charged protamine at 1:5 molar ratio in H₂O. (2) Then this polyplexes nanocore was mixed with negatively charged anionic liposomes to form LP nanoparticles. (3) At the final step, Tf-PEG-DSPE were applied to LPs to form Tf-LPs targeting nanoparticles through a post-insertion process.

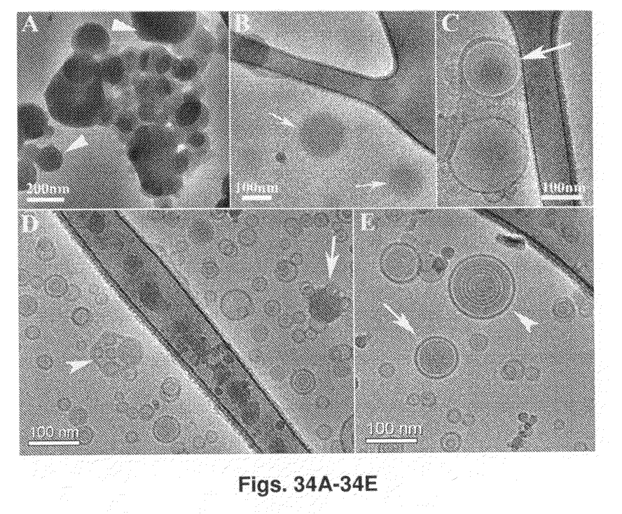

Detailed nanostructures of polyplexes and LPs were studied by direct nanoscale imaging via Cryo-TEM (FIG. 34). Distinct coexisting structures were demonstrated, including an onion-like LP in which the ODNs are condensed between two adjacent lipid bilayers (FIG. 34C).

In FIG. 34D, we demonstrate the diversity in LP morphology. The white arrow shows amorphous complex of protamine/ODN, with small liposomes attached to it. The liposomes fusion to the protamine/ODN complex is probably due to electrostatic attraction between the positively charged protamine/ODN complex and the anionic liposomes. The white arrowhead points to “membrane sac” that contains empty liposomes and onion-like LPs.

In FIG. 34E, the white arrow indicates a structure that is attributed to the CHEMS system without the addition of protamine or ODNs. This structure is composed of an amorphous core and a membrane layer that surrounds it. This inner membrane layer is clearly distinct from the amorphous core by difference in contrast. Also, this core is resolved from an external vesicle that encapsulates it. This structure was also observed in the lipids solution, showing that this structure contains neither protamine nor ODNs.

Another structure is indicated by a white arrowhead in FIG. 34E. This particle consists of lipids bilayers and an outer thick layer of protamine/ODN complex sandwich between two adjacent bilayers. This LP is the result of electrostatic attraction between the protamine/ODN complex and the anionic lipids bilayers. The amorphous complex of protamine and ODNs attaches to the outer surface of the lipid bilayers, at least partially coats the outer surface, and attracts another lipid bilayer to sandwich it.

LPs had an average particle size as 108.5±5.4 nm and a zeta potential as 12.12±0.82 mV. The GTI-2040 encapsulation efficiency was determined by agarose gel electrophoresis and found to be over 90%.

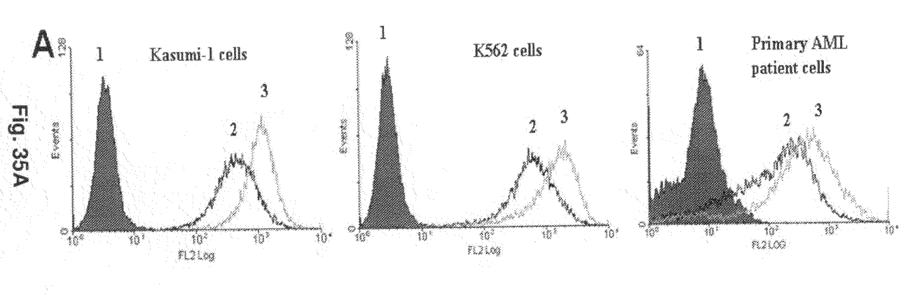

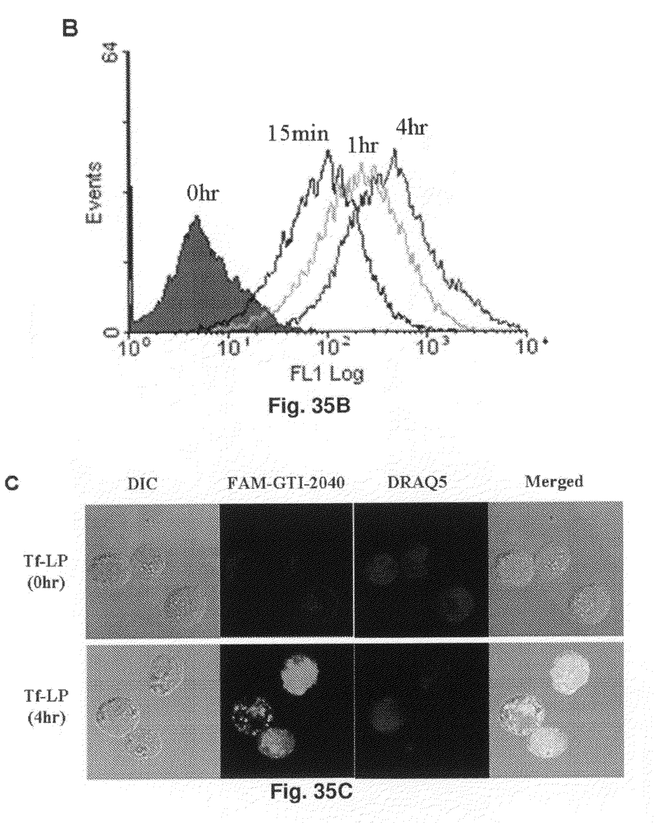

TfR expression on AML cells and patient primary blasts. Tf is the targeting molecule on LPs, which can be efficiently uptaken by cells expressing TfR via TfR-mediated endocytosis (19, 20). TfR is a dimeric transmembrane glycoprotein (180 kea) commonly overexpressed on proliferating cells including most tumor cells, such as leukemia (21, 22). TfR expression on the surface of AML cells was studied using PE-labeled anti-TfR monoclonal antibodies. Kasumi-1 cells, K562 cells and AML patient cells used in this study demonstrated a relatively high level expression of TfR (FIG. 35A). In addition, TfR expression levels on Kasumi-1, K562 and patient primary cells were increased by deferoxamine (DFO) (FIG. 35A), an iron chelator known to increase TfR expression (23).

Cellular uptake of GTI-2040-Tf-LPs in AML cells. In order to study the uptake of GTI-2040-Tf-LPs, AML cells were treated with Tf-LPs containing FAM-labeled GTI-2040. The treated AML cells were collected at various time points and washed twice with PBS before analysis. Flow cytometry analysis of these AML cells showed a time-dependent increase in fluorescence signals (FIG. 35B), indicating the time-dependent cellular uptake of FAM-GTI-2040-Tf-LPs in AML cells. Confocal microscopy confirmed the delivery of FAM-GTI-2040 into AML cells by Tf-LPs (FIG. 35C).

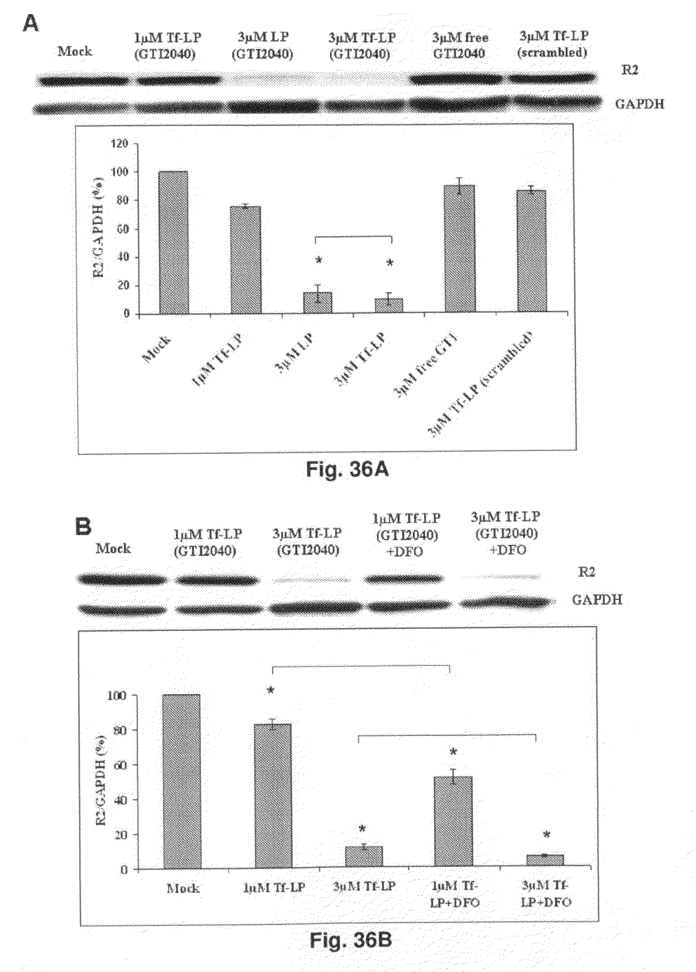

R2 downregulation by GTI-2040-Tf-LPs in AML cells. The efficiency of targeted delivery of GTI-2040 by Tf-LPs was further evaluated based on changes in R2 expression at the mRNA and protein levels in various AML cell lines, such as Kasumi-1 and K562. In Kasumi-1 cells, 25±1% of R2 protein reduction was achieved in cells treated with 1 μM of GTI-2040-Tf-LPs compared to buffer-treated controls. In contrast R2 protein reduction was only 11±6% in cells treated with the non-targeted GTI-2040-LPs. Treatments with 1 μM free GTI-2040, LPs (scrambled ODNs) or Tf-LPs (scrambled ODNs) did not result in any R2 downregulation (data not shown). When the ODN concentration was increased to 3 μM, R2 was further downregulated in cells treated with GTI-2040-Tf-LPs (90±2%) (FIG. 36A). Treatment with 3 μM GTI-2040-LPs induced 84±2% R2 downregulation, and 3 μM Tf-LP (scrambled ODNs) only caused 14±3% R2 downregulation. A similar trend of R2 mRNA downregulation was observed. This shows that the enhanced downregulation of R2 by the GTI-2040-Tf-LPs reflected the enhanced delivery of GTI-2040 into the cells by Tf-LPs, compared to free GTI and scrambled controls.

Delivery of GTI-2040 by Tf-LPs was further enhanced by pre-treating the cells with 30 μM DFO for 18 hr (FIG. 36B) which upregulates TfR expression in AML cells (FIG. 35A). As shown in FIG. 36B, at 1 μM GTI-2040-Tf-LP concentration, DFO pre-treatment improved R2 downregulation (49±4%) in Kasumi-1 cells compared to the untreated samples (17±3%). At 3 μM GTI-2040-Tf-LP concentration, DFO pre-treatment also improved the R2 downregulation from 88±1% to 94±1%.

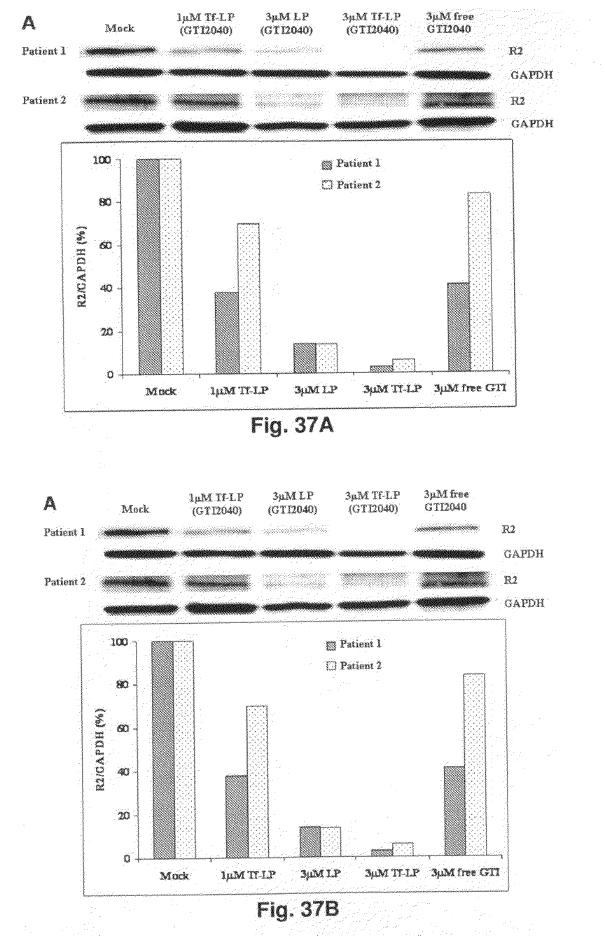

R2 downregulation by GTI-2040-Tf-LPs in AML patient primary cells. Dose-dependent enhancement in R2 downregulation was observed in all the AML patient primary cells tested (FIG. 37). The effect of DFO pre-treatment is shown in FIG. 37B. DFO pre-treatment improved the R2 downregulation effect of GTI-2040-Tf-LPs at both 1 μM and 3 μM concentrations, while DFO pretreatment itself did not show any influence on R2 (FIG. 37B). Scrambled-Tf-LPs did not cause any significant R2 downregulation, suggesting that the improved R2 downregulation in GTI-2040-Tf-LPs treated samples is due to the improved delivery of GTI-2040 into the cells.

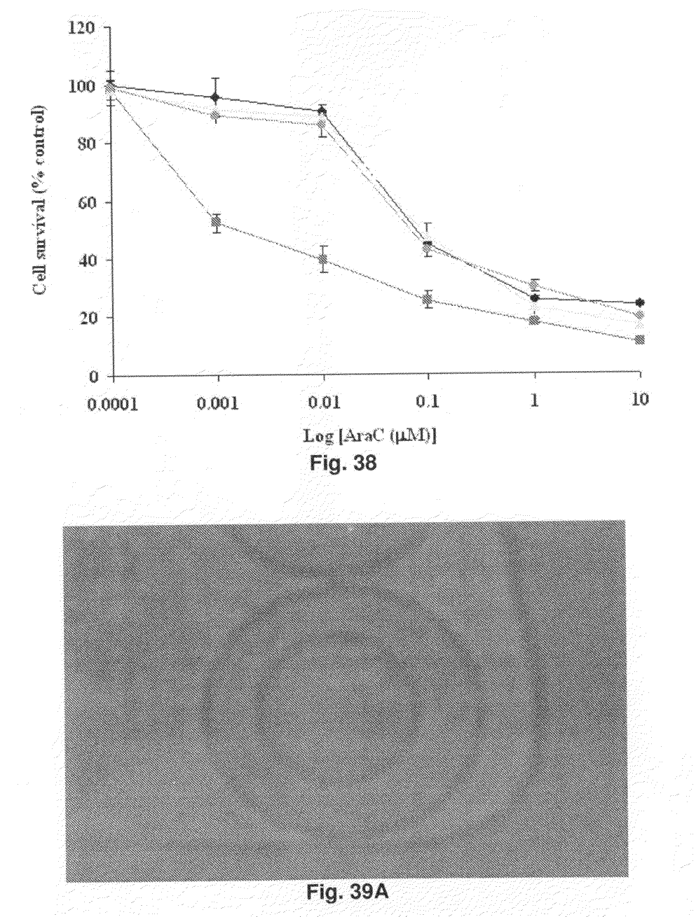

GTI-2040-Tf-LPs improved the chemosensitivity of AML cells to Ara-C. AML cells were treated with GTI-2040-Tf-LPs, free GTI-2040 or Scrambled-Tf-LPs, and then challenged the cells with Ara-C at various concentrations. Cell survival was evaluated by MTS assay. As shown in FIG. 38, at the concentration as low as 1 μM, only GTI-2040-Tf-LPs could sensitize Kasumi-1 cells to Ara-C, with the IC₅₀ of Ara-C decreased by 5 fold from 47.69 nM to 9.05 nM. Free GTI-2040 and Tf-LPs containing scrambled ODN had no chemosensitization effect, consistent with the trend observed for R2 downregulation (FIG. 36A).

### Discussion of Example E

Example E provides show non-limiting examples of formulations capable of promoting targeted delivery of ODNs, thereby enhancing their clinical efficacy and reduce their side effects. Example E shows that Tf-LPs efficiently delivered GTI-2040 into AML cells, downregulated R2, and chemosensitized the cells to chemotherapy agent Ara-C. These effects were highly sequence specific and formulation dependent, as Tf-LPs containing scrambled ODN and free GTI-2040 barely showed any effect. No significant cytotoxicity due to the LP formulation was observed at the concentrations used in Example E.

Overcoming the delivery obstacle is the greatest challenge for ODNs in clinical application (24, 25). A variety of vehicles have been developed to facilitate delivery of ODNs (26). Polymers and lipids are two major classes of materials commonly used for condensing DNA/ODN into nanoparticles by forming polymer-DNA complexes (polyplexes) (27-31), lipid-DNA/ODN complexes (lipoplexes) (32-35), and lipid-polymer-DNA/ODN ternary complexes (LPs) (36-38), respectively.

In Example E, we developed LP nanoparticles for GTI-2040 ODN delivery. The advantage of LPs is that DNA/ODN is optimally stabilized via complex with the cationic polymer which has high charge density. Furthermore, LPs are stabilized with a lipid coating that enables flexible surface modifications such as PEGylation to promote colloidal stability, long plasma half-life, and enhanced permeability and retention (EPR) effect-mediated delivery. Also, targeting ligands such as antibodies (e.g., anti-CD52) (12, 13, 39), Tf (15), and folate (40) have been conjugated to LPs to achieve specific delivery in tumor tissue expressing the corresponding antigens or receptors. The LP formulation platform provides a useful strategy for engineering of targeted multifunctional nanoparticles for ODN delivery, such as GTI-2040, and overcome the delivery problems hitherto faced by these compounds.

Protamine sulfate, a polycationic peptide, was used as a good candidate of biodegradable cationic polymers. It can bind ODNs to form a compact structure via electrostatic interactions, and has been shown to facilitate DNA delivery (41). Lipid bilayers composed of CHEMS, a pH-sensitive lipids, and DOPE (a fusogenic lipid) undergoes a transition from lamellar to hexagonal II phase at low pH, which can destabilize endosomes through proximity following endocytosis (25). Therefore, LPs with these lipids are capable of releasing their contents in response to acidic pH within the endosomal system while remaining stable in plasma, thus improving the cytoplasmic delivery of ODNs after endocytosis. Tf, an 80 kDa iron-transporting glycoprotein, can be efficiently taken up by cells via TfR-mediated endocytosis (19, 20). TfR is considered a good target for cancer-specific delivery, as it is commonly overexpressed in cancer cells including AML (21, 22) compared to normal cells. This was confirmed (FIG. 35A). In addition, Tf is less immunogenic than monoclonal antibodies, cost-effective, and easy to handle and store (42).

The detailed structure of LP nanoparticles was studied with Cryo-TEM, indicating several coexisting structures.

Because of early onset of mechanisms of resistance, AML patients are commonly treated with multidrug chemotherapy regimen. GTI-2040 was combined with Ara-C, which represent the backbone for both upfront and salvage regimen in AML. The rationale for this combination is that the metabolite of Ara-C, Ara-CTP, incorporates into DNA and terminates DNA chain elongation by competing with the endogenous dCTP derived from RNR-mediated nucleotide reduction (43-46). It is believed that downregulation of the R2 subunit of RNR by GTI-2040 decreases the endogenous levels of dCTP and further increases the Ara-CTP/dNTP ratio thereby augmenting DNA incorporation of Ara-CTP (8). This combination has been studied in the phase I clinical trial at OSU, leading to promising results (7). However, the in vivo downregulation of R2 in patients treated on this trial was only approximately 20-30%. Therefore, to attain a more efficient R2 downregulation and further enhance the therapeutic efficacy of GTI-Ara-C combination, we improved the intracellular delivery of GTI-2040 by Tf-LPs. At the concentration of GTI-2040-Tf-LP as low as 1 μM, it could sensitize AML cells to Ara-C, with the IC₅₀ of Ara-C decreased by 5 fold, thereby further showing that this combination is effective.

### Example F

Targeted Delivery of GTI-2501 to KB Cells Using Cationic Lipid nanoparticle. GTI-2501 is a 20-mer oligonucleotide that is complementary to a coding region in the mRNA of R1, the large subunit of ribonucleotide reductase (RNR). RNR is a protein that is essential for DNA synthesis and cell growth in normal cells, where expression of RNR is tightly controlled. Cancer cells, however, highly overexpress RNR, which then contributes to tumor growth and malignancy. Overexpression of RNR also promotes resistance to certain chemotherapy drugs, and RNR cooperates with a variety of cancer-causing oncogenes to further promote cancer progression and metastasis. Current results provide evidence that GTI-2501 acts in a sequence-specific, dose-dependent manner to downregulate R1 with a concomitant decrease in proliferation, tumor growth and metastasis. Despite the exciting opportunities, the clinical application of ODNs has been slow due to several major challenges: rapid clearance in blood circulation, poor cellular uptake, and lack of specific targeting.

In Example F, the in vitro experiment supports that GTI-2501 can efficiently decrease R1 gene expression by this kind of lipid nanoparticle. This provides a new approach to improve the clinical efficacy of both ODNs and cationic lipid nanoparticle-mediated therapy.

Characterization of Cationic Lipid nanoparticle. Cationic lipid nanoparticle size distribution was analyzed by particle sizing systems (Santa Barbara, Calif., USA). Particles without transferrin were 111.8 nm in mean diameter. Particles with transferrin were 277.8 nm in mean diameter. Cationic lipid nanoparticle nanoparticles stayed stable for several weeks in cell culture media containing 50% serum.

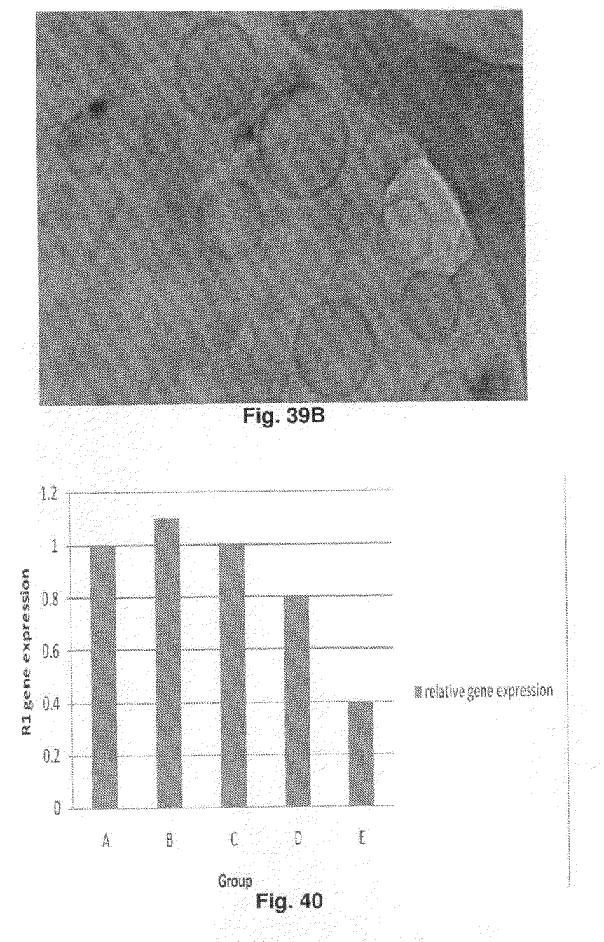

Cryo-TEM examination of thin films of vitrified samples showed that lipid suspensions, at all cholesterol ratios, contained solely lipid nanoparticles. The lipid nanoparticles were unilamellar or oligolamellar, and heterogenous in shape and size. FIG. 39A shows a representative vitrified oligolamellar lipid nanoparticle, with well-defined concentric bilayers. FIG. 39B shows a unilamellar lipid nanoparticle.

Primers Design and Cell Culture. Reverse transcription was performed by using Superscript III first strand synthesis system for RT-PCR (Invitrogen, Carlsbad, Calif.). The housekeeping gene β-actin was used as positive control. The primers used correspond to the following cDNA sequences (the data presented in Table 7 indicate Genebank accession number). The primers were designed by Primer3 tool (v. 0.4.0).

TABLE 7
Sequences of primers used to amplify human
R1 mRNA by reverse transcriptase-polymerase
chain reaction (RT-PCR)

| Gene | Primer | 5′-3′ |
|---|---|---|
| β-actin | Forward | TCC CTG GAG AAG AGC TAC GA |
| β-actin | Reverse | AGC ACT GTG TTG GCG TAC AG |
| R1 | Forward | AAC AAG GTC GTG TCC GCA AA |
| R1 | Reverse | CAT CTT TGC TGG TGT ACT CC |

KB cells are cultured in 6 mm wells and divided into 5 groups according to different culture conditions (Table 8).

TABLE 8
shows the culture condition s of 5 KB cell groups.

| | RPMI- | Lipid | | |
|---|---|---|---|---|
| Group | 160 + serum | nanoparticle | ODN | Tf |
| A | ✓ | x | x | x |
| B | ✓ | ✓ | x | x |
| C | ✓ | x | ✓ | x |
| D | ✓ | ✓ | ✓ | x |
| E | ✓ | ✓ | ✓ | ✓ |

Evaluation of R1 Gene Expression by Cationic Lipid nanoparticle-Mediated GTI-2501 Delivery. Realtime PCR results displayed that treatment with GTI-2501 caused a significant decrease in R1 mRNA, especially when lipid nanoparticle combined with holo-transferrin (FIG. 40).

Example F shows that the strategy described herein is useful to improve the ability of cationic lipid nanoparticle carrier to target cancer cells. Example F also shows that GTI-2501 can inhibit R1 gene expression using the nanocarrier described herein in in vitro experiments. Further, this lipid nanoparticle is determined to be less toxic by realtime PCR. The nanocarriers are also useful to significantly improve the clinic efficacy of anti-cancer therapy, leading to decreased drug dosage and related side-effects.

### Example G

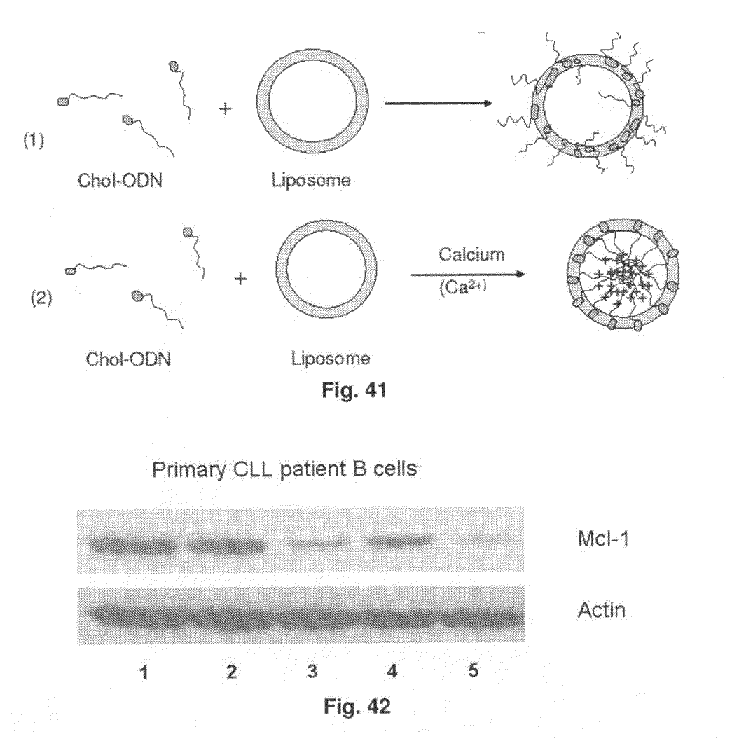

A study of the biological function of LPN-siRNA was conducted in primary chronic lymphocytic leukemia (CLL) B cells. FIG. 41 is a schematic illustration showing strategies for efficiently loading cholesterol modified ODN/siRNAs into liposomal nanoparticles. In particular, the use of calcium provides the advantages of high loading efficiencies, and flexible formulation compositions that can be neutral, anionic or cationic.

FIG. 42 shows enhanced Mcl-1 down-regulation by LPN-Mcl-1 siRNA formulation with Calcium (#5), compared to the formulation without Calcium (#4) and the negative siRNA control (#4). Additionally, LPN formulated Mcl siRNAs work more efficiently than free Mcl-1 siRNA (#2). In FIG. 42, 1. Mock; 2. Free Mcl-1 siRNA; 3. LP (no Ca2+, Mcl-1); 4. LP (no Ca2+, Negative); 5. LP (Ca2+, Mcl-1).

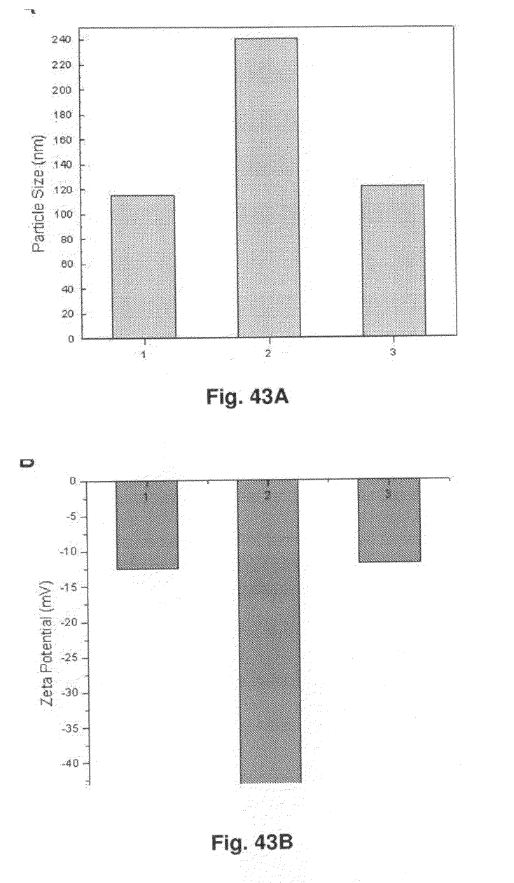

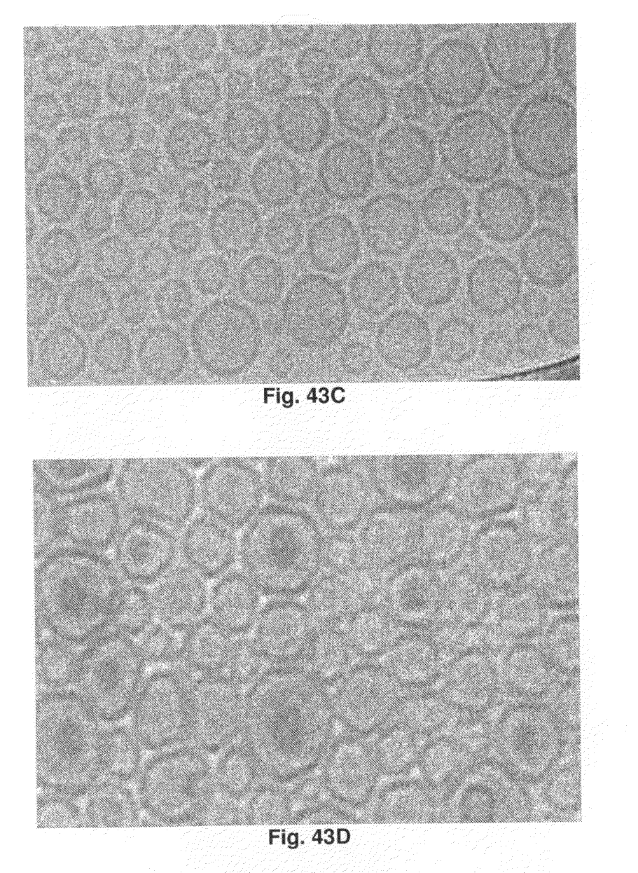

A study of liposomal nanoparticle containing cholesterol-modified oligonucleotides by using neutral lipids was conducted. FIGS. 43A-43B show the changes of particles size after introducing calcium (FIG. 43A) and surface charge (zeta potential) (FIG. 43B) where the formulation is EggPC/Chol/PEG-DSPE —70/28/2, lipids/OND 10/1; where #1 is Lipid nanoparticle alone; #2 is LP containing Chol-ODN; (no Ca2+); and #3 is LP containing Chol-ODN and Ca2+ (10 mM). FIG. 43C shows a CryoTEM of Chol-ODN Encapsulated Lipid nanoparticles without Ca2+ where the formulation is EggPC/Chol/PEG-DSPE—70/28/2, lipids/OND 10/1. FIG. 43D shows a CryoTEM of Chol-ODN Encapsulated Lipid nanoparticles with Ca2+ where the formulation is EggPC/Chol/PEG-DSPE —70/28/2, lipids/OND 10/1.

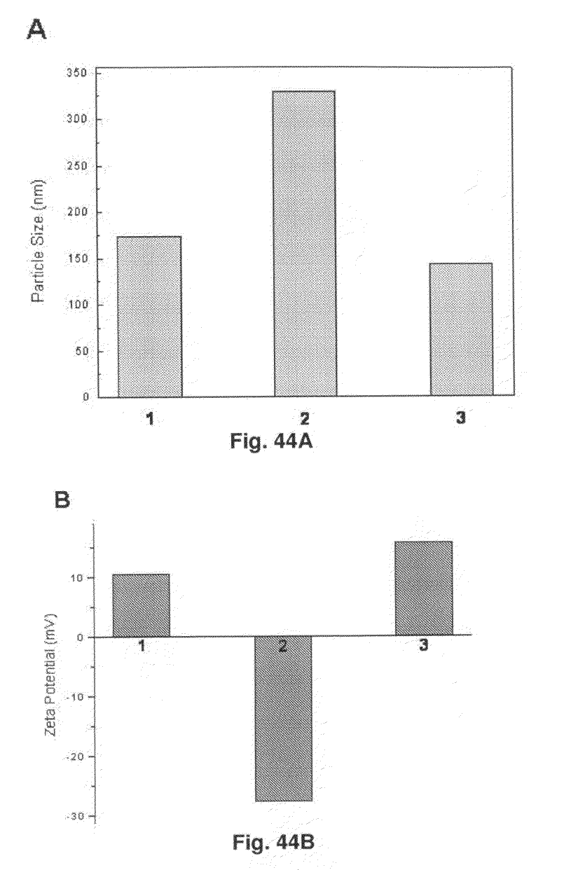

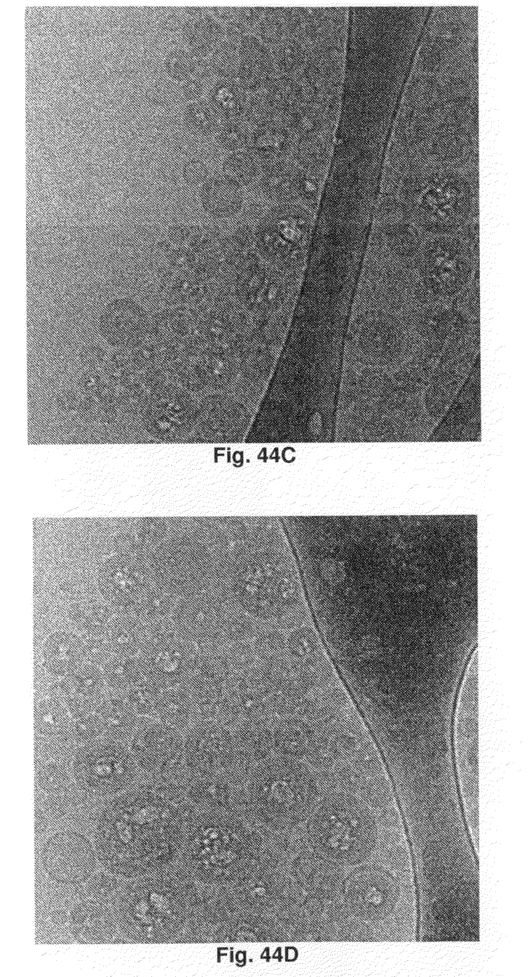

A study of liposomal nanoparticle containing cholesterol-modified oligonucleotides by using neutral lipids was conducted. FIGS. 44A-44B show the changes of particles size after introducing calcium (FIG. 44A) and surface charge (zeta potential) (FIG. 44B) where the formulation is DC-chol/EggPC/PEG-DSPE —33.5/65/1/5, lipids/OND 10/1; where #1 is Lipid nanoparticle, ODN; #2 is LP containing Chol-ODN; (no Ca2+); and #3 is LP containing Chol-ODN and Ca2+ (5 mM). FIG. 44C shows a CryoTEM of Chol-ODN Encapsulated Lipid nanoparticles without Ca2+ where the formulation is DC-chol/EggPC/PEG-DSPE—33.5/65/1/5, lipids/OND 10/1. FIG. 44D shows a CryoTEM of Chol-ODN Encapsulated Lipid nanoparticles with Ca2+ where the formulation is DC-chol/EggPC/PEG-DSPE—33.5/65/1/5, lipids/OND 10/1.

### Example H

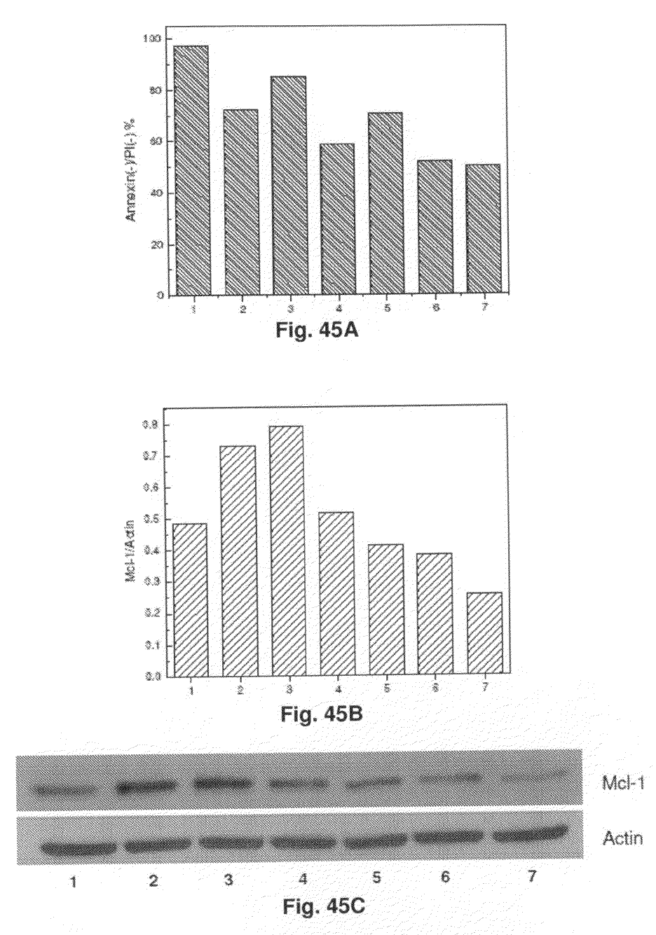

FIGS. 45A-45C show Mcl-1 down regulation in Raji cells by siRNA delivered via anti-CD20 conjugated nanoparticles (CD20 ILP) in CLL patient cells. #1. Mock; #2. LP (Mcl-1, 100 nM); #3. LP (negative, 100 nM); #4. CD37 ILP (Mcl-1, 100 nM); #5. CD37 ILP (negative, 100 nM); #6. CD20 ILP (Mcl-1, 100 nM); #7. CD20 ILP (negative, 100 nM).

FIG. 45A shows the percentage of live Raji cells was determined by Annexin V/PI staining and was analyzed by flow cytometry. FIG. 45B is a graph showing Mcl-1/Actin for #1-#7. FIG. 45C shows the Western blot analysis of Mcl-1 protein and β-actin

### Example I

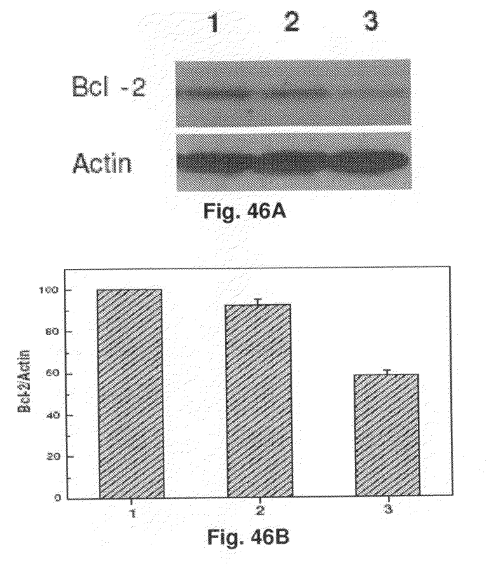

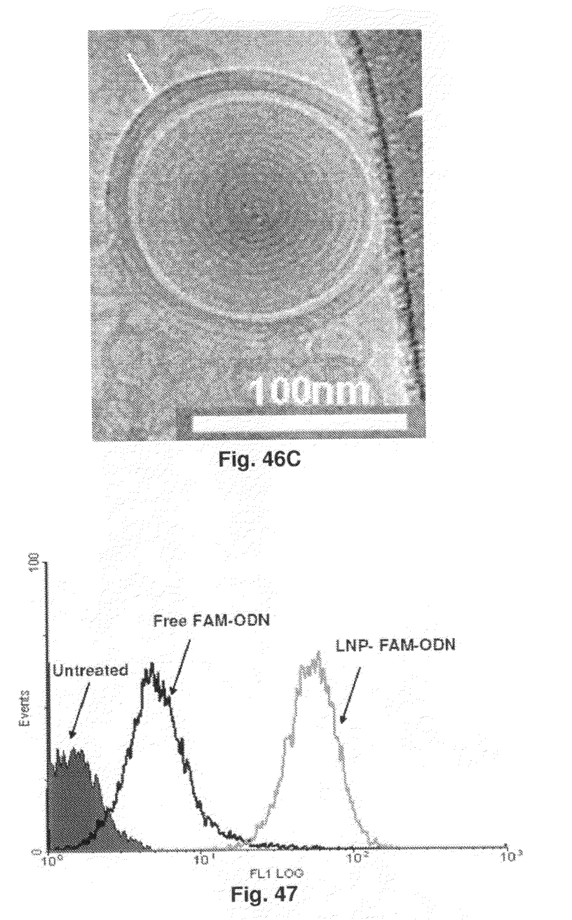

Analysis of bcl-2 protein down-regulation by free G3139 (Bcl-2 anti-sense ODN) and LNP-G3139 on K562 human leukemia cells. K562 cells were treated with free 1 uM G3139 or LNP formulated G3139 for 48 hrs. FIG. 46A represents the western blot expressions of Bcl-2 protein and β-actin loading control. FIG. 46B represents the RT-PCR analysis of Bcl-2 mRNA level. In FIG. 46B, results present as means of n=3 independent experiments. LNP Formulation: DC-Chol/EggPC/PEG-DSPE=30/68/2 (molar ratio) and lipids/ODN/protamine=12.5/1/0.3 (weight ratio). The data showed that the LNP-formulated antisense ODN has much greater biological activity. FIG. 46C shows the cryoTEM image the structure of oligonucleotide-lipid nanoparticles. The coexistence of a two-layer lipid membrane (arrow) and a condensed multilamellar polyplexes is shown. The formulation of ODN-lipid nanoparticles is DC-Chol/EggPC/mPEG-DSPE=30/68/2 (molar ratio) and lipids/ODN/protamine=12.5/1/0.3 (weight ratio).

### Example J

FIG. 47 shows the increased uptake of nanoparticle (LNP) formulated FAM-ODN (fluorescein-labeled ODN) by Raji Burkett's Lymphoma cells. Raji cells were incubated with free ODN, LNP-FAM-ODN at 1 uM at 37° C. for 1.0 hr and washed twice with cold PBS. The cells were analyzed by flow cytometry to measure cell-associated FAM-ODN fluorescence. Untreated cells were used as a negative control. LNP formulation: DC-Chol/EggPC/mPEG-DSPE=33.5/65/1.5 (molar ratio) and total lipids/ODN/protamine=12.5/1/0.3 (weight ratio). The data showed that the LNP formulated ODN was taken up more efficiently than the free ODN.

### Example K

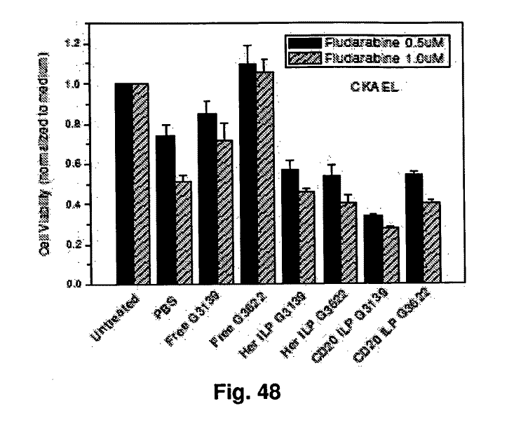

The therapeutic efficacy of antibody-targeted nanoparticles (ILPs) is shown in FIG. 48. Leukemia cells from patients with chronic lymphocytic leukemia (CLL) were treated with either controls or anti-CD20 antibody conjugated lipid nanoparticles (CD20 ILP) loaded with antisense ODN G3139, combined with chemotherapy drug fludarabine. The data showed that the antibody-targeted nanoparticles were very effective in making the leukemia cells more sensitive to the chemotherapy drug fludarabine, which is an indication that antibody mediated specific targeting enhanced the delivery of the oligonucleotide.

### Example L

In another non-limiting Example, the LP are synthesized by a microfluidic focusing method which is useful to improve the uniformity of the nanoparticle size and structure, as well as increase ODN loading with less lipids and condensing agents for better transfection efficiency and less cytotoxicity.

A microfluidic hydrodynamic focusing (MF) system to prepare lipopolyplex (LP) containing antisense deoxyoligonucleotide (G3139, oblimerson sodium, or Genasense™), for targeting Bcl-2, an antiapoptotic protein commonly overexpressed in numerous cancers was developed. The lipopolyplex consist of ODN:protamine:lipids (1:0.3:12.5 wt/wt ratio) and the lipids included DC-Chol:egg PC:PEG-DSPE (40:58:2 mol/mol %). Using k562 human erythroleukemia cells, which contain an abundance of Bcl-2 and overexpression of transferrin receptors (TfR), and G3139 as a model cell line and drug, respectively, the Bcl-2 downregulation at the mRNA and protein levels were compared between conventional bulk mixing (BM) method and microfluidic hydrodynamic focusing (MF) method, in addition to cellular uptake and apoptosis. The lipopolyplex size and surface charge was characterized by dynamic light scattering (DLS) and zeta potential (ξ) measurement while the ODN encapsulation efficiency was determined by gel electrophoresis. Cryogenic transmission electron microscopy (Cryo-TEM) was used to determine the morphology of the LPs. These results demonstrated that MF produced LP nanoparticles had smaller size and size distribution but with similar morphology. Furthermore, MF LP nanoparticles more efficiently downregulated Bcl-2 protein level than BM LP nanoparticles with or without conjugating LPs with transferrin.

### Introduction for Example L

The in vivo application of therapeutic molecules (free/naked plasmids or ODNs) are limited by rapid clearance from blood circulation, lack of selectivity for target cells, low permeability through the cell membrane, and degradation by serum nucleases. To overcome these limitations, plasmids or ODNs have been complexed with polymers or lipid nanoparticles. Lipid nanoparticles are self-assembling vesicles that can encapsulate hydrophilic drugs in their interior aqueous core, whereas lipophilic and amphiphilic drugs can be embedded in the lipid bilayers.

In Example L, we demonstrate strategy for nanoparticle manufacturing based on microfluidic technology. By precisely controlling the flow conditions and mixing process of the reagents at the micrometer scale, nanoparticles with uniform and well-defined size, structure, and pharmacological functions are synthesized. These nanoparticles are especially useful for efficient delivery of DNA oligonucleotide compounds to cancer cells.

In one embodiment, one or more of the following are incorporated into the nanoparticles: protamine, which stabilizes ODN in serum and increases delivery efficiency; transferrin which shields LPs from the serum proteins and for targeting transferrin receptors (TfR); and PEG-DSPE which further stabilizes the LPs against plasma protein adsorption and clearance by the RES. The method provides a stable lipopolyplex (LP) formulation that yields nanoparticles of sizes less than about 150 nm, high ODN entrapment efficiency, colloidal stability, long circulation time, and specific targeting to cancerous cells.

The lipopolyplex (LP) nanoparticles, i.e. lipid nanoparticles containing DNA, are assembled in the microdevice specifically for delivery into cancer cells.

Materials and Methods.

Egg phosphatidylcholine (egg PC), 3β-[N-(N′,N-dimethylaminoethane)-carbamoyl] cholesterol (DC-Chol) and distearoyl phosphatidylethanolamine-N-[maleimide-polyethylene glycol, M.W. 2000] (Mal-PEG-DSPE) were purchased from Avanti Polar Lipids (Alabaster, Ala.). Methoxy-PEG2000-DSPE (PEG-DSPE) was purchased from Genzyme Corporation (Cambridge, Mass.). Human holo-transferrin (Tf), 2-iminothiolane (Traut's reagent), protamine sulfate, and other chemicals and reagents were purchased from Sigma (St. Louis, Mo.). All tissue culture media and supplies and M-murine leukemia virus reverse transcriptase were purchased from Invitrogen (Carlsbad, Calif.). RNeasy mini kit, RNAse inhibitor, and Float-A-Lyzer were purchased from Qiagen (Valencia, Calif.), Promega (Madison, Wis.), and Spectrum Labs (Rancho Dominguez, Calif.), respectively.

Antisense oligonucleotides. All ODNs used in this study were fully phosphorothioated. Antisense ODN G3139 (5′-TCT CCC AGC GTG CGC CAT-3′) [SEQ ID NO:1] and its fluorescence-labeled derivative, FITC-03139 (G4243).

Microfluidic devices design and fabrication. Plastic microfluidic devices were fabricated. The microfluidic hydrodynamic focusing (MF) devices were designed in AutoCAD (Autodesk, San Rafael, Calif.) and a g-code program was generated and then transferred into a high precision computer numerically controlled (CNC) machine (Aerotech, Inc.) which was used to machine the patterns on a poly(methyl methacrylate) (PMMA) plate. The channel widths were varied by using the appropriate end mill sizes. A 45 μm thick PMMA film was thermally laminated to form the closed channels by passing the PMMA/film sandwich through a thermal laminator (GBC, Inc.). Prior to thermal bonding, the microchannels were gently brushed to remove any debris and then the PMMA plates were sonicated in IPA/DI H2O (1:10) for 5-10 min to remove grease and then blown dry. After lamination, fluidic connectors were bonded onto the PMMA plate by applying a UV curing adhesive around the perimeter of the connectors. The connectors were aligned over the inlet/outlet openings and the adhesive was cured by exposure to UV irradiation (Novacure 2100, EFXO Corp., Quebec, Canada) for 10 sec. The assembled devices were sterilized overnight under UV light in a cell culture hood prior to experimentation.

The MF device consists of three inlet ports and one outlet port. The inlet ports are each connected to sterile syringes containing protamine or lipids or protamine/lipids or ODN solution. At inlet port 1 or 2, a fluid stream was introduced into each port that split into 2 side microchannel streams (microchannels a and c or e and f) while at inlet port 3, a fluid stream was introduced in the center microchannel (microchannel b). The products stream was collected at the outlet port (microchannel g). Two flow configurations were used to produce LPs as shown in Table 9. The protamine (microchannels a and c) and lipids (microchannels e and f) or protamine/lipids streams (microchannels a and c or e and f) would be injected first and then the ODN stream. After the ODN stream has entered and the hydrodynamic focusing established, the products were flowed for a further 3-5 min to allow for steady state before being collected in sterile tubes at the outlet port (microchannel g). The magnitude of the hydrodynamic focusing was controlled by altering the flow rate ratio (FR) of the side streams to the middle stream. FR is the ratio of total flow rate to the middle stream flow rate. Two programmable syringe pumps (Pump 33, Harvard Apparatus, Holliston, Mass.) were used to control the fluid flow rates independently. For flow visualization, the MF device was mounted on an inverted microscope stage (Nikon Eclipse 2000U) with a 10× Nikon Plan Fluoro objective.

Cell culture. All cells, purchased from American Type Culture Collection (ATCC) (Manassas, Va.), were cultured in RPMI 1640 media supplemented with 10% heat-inactivated fetal bovine serum (FBS), 100 U/mL penicillin, 100 μg/mL streptomycin, and L-glutamine at 37° C. in a humidified atmosphere containing 5% CO₂.

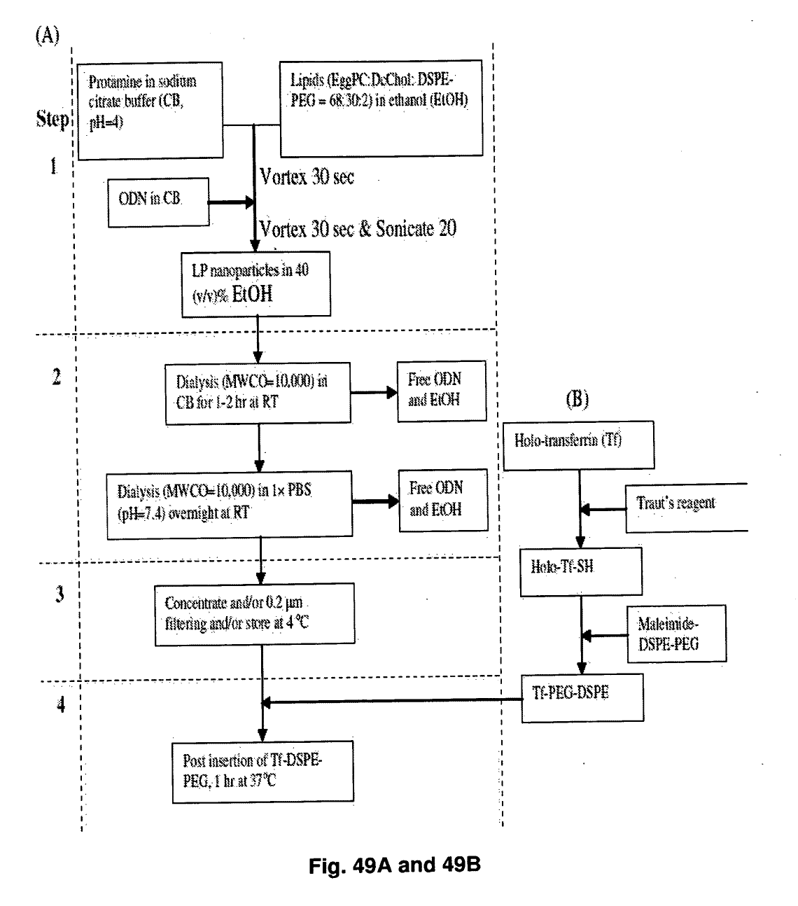

Preparation of transferrin conjugated PEG-DSPE (Tf-PEG-DSPE) and Tf-receptor targeted G3139-containing LPs (Tf-LP). Transferrin was conjugated to PEG-DSPE. Briefly, holo (diferric)-transferrin (holo-Tf) in 1× phosphate-buffered saline (PBS, pH=8) was reacted with 5× Traut's reagent to yield thiolated Tf (holo-Tf-SH). Free Traut's reagent was removed through column separation with 1× phosphate-buffered saline (PBS, pH=6.5) using protein assay (Bio-Rad) to detect Tf in the elution. Holo-Tf-SH was then reacted with micelles of Mal-PEG-DSPE at a molar ratio of protein-to-lipid of 1:10 for 2 h at room temperature in 1×PBS (pH=6.5) and dialyzed using a SpectraPor Float-A-Lyzer MWCO 5,000 Dalton (Spectrum Labs, Rancho Dominguez, Calif.) against 1×PBS (pH=7.4) to form Tf-PEG-DSPE as shown in FIG. 49.

A post-insertion method was adopted to incorporate T_{f} ligand into ODN-loaded LPs. ODN-loaded LPs were incubated with Tf-PEG-DSPE for 1 hour at 37° C. at Tf-PEG-DSPE-to-LP lipid ratio of 1:100 (1 mol % based on DSPE-PEG) to form Tf-LPs.

Preparation of G3139-containing LPs by bulk mixing (BM) and microfluidic hydrodynamic focusing (MF) methods. An ethanol dilution method was used to prepare the LPs containing G3139. For the BM method as shown in FIG. 49, a lipid mixture (egg PC:DC-Chol:PEG-DSPE at molar ratio 68:30:2) in absolute ethanol (EtOH) was mixed with protamine sulfate in sodium citrate buffer (20 mM, pH=4) at a mass ratio and a volume ratio of lipid-to-protamine sulfate of 12.5:0.3 and 2:1, respectively, to obtain an EtOH concentration of 66.6% (v/v). ODN, dissolved in sodium citrate buffer (20 mM, pH=4) was then added into the lipid/protamine solution followed by vortexing for 30 sec to spontaneously form pre-LPs at EtOH concentration of 40% (v/v) where the weight ratio of ODN:protamine:lipids was 1:0.3:12.5.

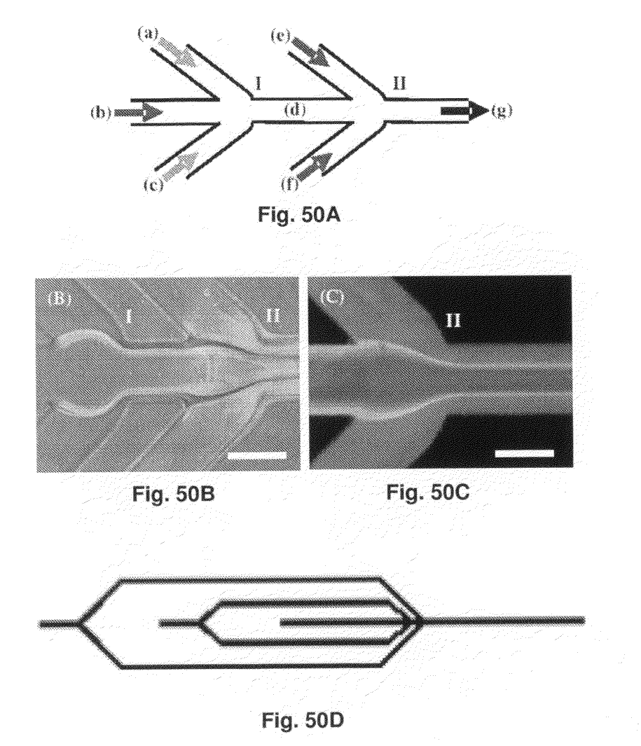

For the MF method, as shown in FIG. 50, a 5-inlet MF system was developed and used to produce the LPs. The MF device consists of 3 inlet ports and 1 outlet port. At inlet port 1 or 2, a fluid stream was introduced into each port that split into 2 side streams while at inlet port 3, a fluid stream was introduced in the center stream. Two flow configurations were tested as shown in Table 9.

TABLE 9
Flow configuration.

| | | 1^{st} | | | | | |
|---|---|---|---|---|---|---|---|
| | 2^{nd} | inlet (b) | 3^{rd} | | 4^{th} | 5^{th} | Outlet |
| Microchannel | inlet (a) | (center) | inlet (c) | (d) | inlet (e | inlet (f) | (g) |
| First | Protamine | ODN | Protamine | | Lipids | Lipids | |
| configuration | | | | | | | |
| Second | ODN | Lipids/ | ODN | | Lipids/ | Lipids/ | |
| configuration | | protamine | | | protamine | protamine | |

For the first configuration, at junction I, an ODN solution stream was introduced in the center microchannel, b, while two protamine sulfate solution streams were introduced in the side microchannels, a and c, to hydrodynamically focus the ODN into a narrow stream to form ODN/protamine nanoparticles or “proticles” via electrostatic interaction between negatively charged ODN and positively charged protamine sulfate. Immediately downstream (˜200 μm) at junction II, another two lipids streams were introduced in the side microchannels, e and f, to further sandwich and squeeze the ODN/protamine streams to form ODN/protamine/lipids nanoparticles or lipopolyplexes. The final weight ratio of ODN:protamine:lipids was 1:0.3:12.5 and the ethanol concentration was 40%. The flow rates for ODN, protamine, and lipids streams were 20, 20, and 450 μL/min, respectively, and were controlled independently by two syringe pumps (Pump33, Harvard Apparatus, Holliston, Mass.). Both ODN and protamine were prepared in sodium citrate buffer (20 mM, pH 4) whereas the lipids mixture was in 100% ethanol.

For the second flow configuration, at junction I, a protamine/lipids mixture stream was introduced in the center microchannel, b, and sandwiched by two ODN side streams, a and c; and immediately downstream (˜200 μm) at junction II, another two protamine/lipids streams, e and f, were introduced to further sandwich and squeeze the ODN/protamine/lipids streams. Again, the final weight ratio of ODN:protamine:lipids was 1:0.3:12.5 and the ethanol concentration was 40%. The flow rates for protamine/lipids, ODN, and protamine/lipids streams were 200, 20, and 200 μL/min, respectively, and were controlled independently by two syringe pumps (Pump33, Harvard Apparatus, Holliston, Mass.).

The pre-LPs produced by both methods vortexed for 30 sec and then sonicated for 20 min followed by dialyzing against sodium citrate buffer (20 mM, pH=4) for 1-2 hour and then in 1×PBS (pH=7.4) overnight at room temperature, using a SpectraPor Float-A-Lyzer MWCO 10,000 Dalton to raise the pH to neutral in order to remove unbound ODN, reduce ethanol, and to partially neutralize the cationic DC-Chol.

For LPs and Tf-LPs containing FITC-labeled ODN (G4243) was used in the preparation of LPs. After dialysis, the LPs were sterilized by filtering through 0.2 μm PVDF filter and stored at 4° C. until further use.

Particle sizes and zeta potentials (ξ). The particle sizes and zeta potentials (ξ) of non-targeted and targeted LPs were analyzed on BI-200SM and ZetaPALS (Brookhaven Instruments Corp., Holtsville, N.Y.), respectively. Volume-weighted Gaussian distribution analysis was used to determine the mean LP diameter and the standard deviation. Each data represents mean±standard deviation of four separate experiments.

ODN encapsulation efficiency. To determine ODN encapsulation, ODN-LP after dialysis was diluted in 1×TE or lysed in 1% sodium dodecyl sulfate (SDS), heated at 95° C. for 5 min in a thermal cycler, then mixed with gel-loading solution at a ratio of 1:5 (Sigma), and loaded on 3% ReadyAgarose gel plus ethidium bromide (Bio-Rad Laboratories, Hercules, Calif.). Electrophoresis was carried out at 100 V for 45-60 min in a 1×TAE running buffer (Invitrogen). A digital image of the gel was captured under UV light using ChemiDoc XRS system (Bio-Rad). The encapsulation efficiency of ODN in the LP was calculated based on the ratio of the amount of ODN before and after SDS treatment and against a standard curve of ODN concentrations.

Cryogenic transmission electron microscopy (cryo-TEM) of LPs. Cryogenic transmission electron microscopy (cryo-TEM) imaging was performed. Briefly, samples were examined in a Philips CM120 microscope (Eindhoven, The Netherlands) operated at 120 kV, using an Oxford CT-3500 cooling holder and transfer station (Abingdon, England). Specimens were equilibrated in the microscope below −178° C., then examined in the low-dose imaging mode to minimize electron beam radiation damage, and recorded at a nominal underfocus of 2-4 μm to enhance phase contrast. Images were acquired digitally by a Gatan MultiScan 791 cooled charge-coupled device camera (Pleasanton, Calif.) using the Digital Micrograph 3.1 software package. Cryo-TEM analysis was performed at Technion-Israel Institute of Technology, Haifa, Israel.

Transfection studies. Leukemia cells were plated in 6-well tissue culture plates at 10⁶/well in 1.2 mL RPMI1640 medium containing 10% FBS. An appropriate amount of Tf-LPs or one of the other formulations was added into each well to yield a final ODN concentration of 1 μM. The cells were then incubated at 37° C. in a CO₂ incubator for 6 hours. The cells were washed, transferred to fresh medium, incubated for another 24 to 48 hours, and then analyzed for bcl-2 mRNA level and Bcl-2 protein level by real-time RT-PCR and Western blot, respectively. All transfection experiments were performed in RPMI1640 medium containing 10% FBS.

Quantification of bcl-2 mRNA level by real-time RT-PCR. The bcl-2 mRNA level in leukemia cells was evaluated using real-time RT-PCR as follows. Total RNA was extracted using RNeasy Mini kit (Qiagen) in accordance to the manufacturer's protocol and concentrations were measured at O.D. 260 nm using a spectrophotometer (Thermo Fisher Scientific, Waltham, Mass.). For cDNA synthesis, 2 μg of total mRNA from each sample was mixed with 1.5 μL, of 20 μM random hexamer and nuclease free water to a total volume of 17 μL. and heated to 70° C. for 5 minutes followed by cooling on ice for at least 5 minutes. 12.9 μL, of master mixture containing 5× reaction buffer, 100 mM dithiothreitol, 10 mM of each dNTP, M-murine leukemia virus reverse transcriptase, and RNAse inhibitor was added into each sample and the samples were then incubated in a thermal cycler (Bio-Rad Laboratories, Hercules, Calif.) at 42° C. for 60 minutes followed by 94° C. for 5 minutes. The resulting cDNA was amplified by real-time PCR iQ5 (Bio-Rad Laboratories, Hercules, Calif.). The following oligonucleotides primers designed by the Primer Express program (Applied Biosystems) were used: Bcl-2, forward and reverse primers were CCCTGTGGATGACTGAGTACCTG [SEQ ID NO:2] and CCAGCCTCCGTTATCCTGG [SEQ ID NO:3], respectively.

Each cDNA sample was used as a template in two separate PCR amplification reactions prepared in a SYBR Green (BioRad) mastermix: (a) a set of primers for Bcl-2 transcripts, and (b) primers for a housekeeping gene ABL. The housekeeping gene ABL mRNA was used as an internal control. bcl-2 mRNA was normalized to ABL mRNA levels.

Quantification of Bcl-2 protein by Western blot. Western blot was carried out to evaluate the Bcl-2 protein level. Untreated and ODN-treated cells were incubated with a lysis buffer containing a protease inhibitor cocktail III (CalBiochem, San Diego, Calif.) on ice for 20 min followed by sonication and centrifugation of the cell lysate at 13,200 rpm and 4° C. for 10 min. Then the supernatant was collected and the protein concentrations were determined by BCA assay (Pierce, Rockford, Ill.) on a spectrophotometer. An aliquot of 100 protein from each sample was loaded onto a 15% Ready Gel Tris-HCl polyacrylamide gel (Bio-Rad, Hercules, Calif.) for 2 hr at 100 V, followed by transfer of the proteins to a PVDF membrane overnight. After blocking with 5% non-fat dry milk in 1× Tris-buffered saline/Tween-20 (TBST) for 1 h, the membranes were incubated with monoclonal mouse anti-human Bcl-2 (Dako, Carpinteria, Calif.) or polyclonal goat anti-human actin antibody (Santa Cruz Biotechnology, Santa Cruz, Calif.) also in 5% non-fat dry milk in TBST. After 2 h of incubation at room temperature (or at 4° C. overnight), membranes were washed 4 times (15 min each) with TBST, followed by incubation with horseradish peroxidase-conjugated sheep antimouse IgG (Amersham Biosciences, Piscataway, N.J.) or rabbit antigoat IgG (Pierce, Rockford, Ill.) in 2.5% non-fat dry milk in TBST for 1 h at room temperature. Membrane was then developed with ECL (GE Healthcare, United Kingdom) or Pierce SuperSignal West Dura Extended Duration Substrate (Pierce, Rockford, Ill.) and imaged with Kodak X-OMAT film (Kodak, Rochester, N.Y.). Bcl-2 protein expression levels were quantified by ImageJ software (NIH Image, Bethesda, Md.) and normalized to the β-actin level from the same sample.

Cellular uptake of FITC-labeled ODN containing LPs analyze by flow cytometry (FCM). Cellular uptake of FITC-labeled ODN (G4243) LPs and Tf-LP was evaluated by incubating 3×10⁵ cells with 0.5 μM FITC-ODN LPs or Tf-LPs in RPMI1640 medium containing 10% FBS for 6, 24, and 48 h at 37° C. and 5% CO₂ in an incubator. The cells were collected by centrifugation, washed twice with cold 1×PBS (pH=7.4), and fixed in 4% paraformaldehyde. As negative control, cells were treated with 1×PBS (pH=7.4). The uptake of FITC-ODNs was observed by fluorescence microscope and quantified by flow cytometry. All measurements were carried out in triplicates to determine the mean fluorescence intensity and the standard deviation (MFI±SD).

Annexin V-FITC staining analyze by flow cytometry (FCM). K562 cells (1×10⁶) were treated with different formulations at a concentration of 1 μM in serum containing medium at 37° C. for 72 h. Cells were washed once with PBS and resuspended in PBS. Cells were then stained with Annexin V-FITC using a kit (BD Biosciences Pharmingen, San Jose, Calif.). Early apoptotic cells bound to Annexin V-FITC but excluded propidium iodide (PI). Cells in late apoptotic stages were labeled with both Annexin V-FITC and propidium iodide. Cells stained with Annexin V-FITC and PI were detected and quantified by flow cytometry (Becton-Dickinson, Heidelberg, Germany) (Ex=488 nm, Em=530 nm) using FITC signal detector (FL1) and PE emission signal detector (FL2), respectively. Results were processed using the Cellquest software (Becton-Dickinson) based on a percentage of total gated cells (10⁴ cells).

Statistical analysis. Data were represented as mean±standard deviations (S.D.) and analyzed by two-tailed Student's t-test using JMP software (Cary, N.C.). p<0.05 was considered statistically significant.

### Results for Example L

Microfluidic device, LP production setup, and flow pattern. A 5-inlet polymeric MF system to produce LP nanoparticles was designed and fabricated as shown in FIG. 50, having 5 inlet microchannels (a, b, c, e, and f) and 1 outlet microchannel (g). During experiments, the MF device was mounted on an inverted microscope to ensure that there were no air bubbles that might disrupt the flow pattern and the flow was at steady state before samples were collected. FIG. 50B shows an optical micrograph of the experimental flow pattern at junctions I and II of the MF system. To visualize the flow pattern, fluorescein and rhodamine were introduced into the microdevice at various flow rates. FIG. 50C shows a typical fluorescence micrograph of flow pattern at junction II where the volumetric flow rates used for rhodamine, fluorescein, and rhodamine were 200, 20, and 200 μL/min, respectively. The green and red colors are fluorescein and rhodamine, respectively.

LP nanoparticles size, zeta potential, and morphology. The average particle size was measured by dynamic light scattering (DLS). For BM method, mixing ODN and protamine in sodium citrate buffer resulted in large aggregates (data not shown).

For the first flow configuration, the flow rates of ODN, protamine sulfate, and lipids were 20, 20, and 450 μL/min (FRR=24.5), respectively. The LP nanoparticle size was 236.9±2.5 nm. Increasing the lipids stream flow rate to 600 μL/min (FRR=32) resulted in only a slight decrease in the particle size to 205.0±5.6 nm.

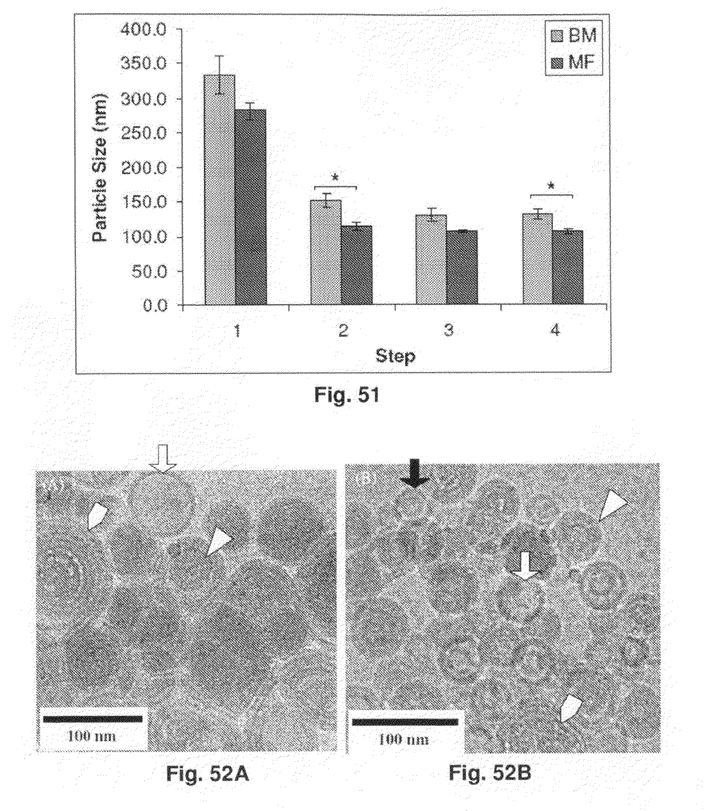

For the second flow configuration, the average particle size was also measured by dynamic light scattering (DLS) at each step in the LP synthesis process by BM and MF methods as shown in FIG. 51. The MF method produced LP nanoparticles that were smaller in size in all the steps; before dialysis (step 1), after dialysis but before filtering (step 2), after dialysis and filtering (step 3), and after post insertion of Tf-PEG-DSPE (step 4).

Table 10 shows the particle size and zeta potential of the LP. The average particle size for BM and MF lipopolyplex before and after post insertion of Tf-PEG-DSPE were 131.0±21.0 nm and 126.7±18.5 and 106.8±5.5 nm and 107.1±8.0 nm, respectively. The zeta potential of the BM and MF LP nanoparticles before and after post insertion were +11.6±3.6 mV and +7.9±1.3 mV and +3.6±2.9 mV and +2.5±4.2 mV, respectively. The decrease in zeta potential indicated that the Tf-DSPE-PEG was successfully incorporated into the LP nanoparticles. Each data represents mean±standard deviation of four separate experiments and p<0.05 is indicated by * symbol.

TABLE 10
Nanoparticle characterization - DLS & zeta potential

| | BM | | MHF | |
|---|---|---|---|---|
| | Mean | Zeta | Mean | Zeta |
| | Particle | Potential | Particle | Potential |
| Method | Size (nm) | (mV) | Size (nm) | (mV) |
| Before dialysis | 334.2 ± 63.6 | — | 282.0 ± 24.0 | — |
| After dialysis | 152.7 ± 22.1 | — | 114.8 ± 12.7 | — |
| After filtering | 131.0 ± 21.0 | 11.6 ± 3.6 | 106.8 ± 5.5 | 7.9 ± 1.3 |
| After post | 131.5 ± 16.1 | 3.6 ± 2.9 | 107.1 ± 8.0 | 2.5 ± 4.5 |
| insertion | | | | |

Mean ± SD (n = 4)

The morphology of LP cannot be easily visualized by optical microscopy and atomic force microscopy (AFM). Therefore, the LP morphology was characterized using cryogenic transmission electron microscopy (Cryo-TEM) where the frozen hydrated samples can be imaged directly with high spatial resolution in their native morphology since the LPs are embedded in a thin film of vitreous ice. The samples were vitrified within 96 hrs after preparation and imaged within 14 days.

As shown in FIG. 52, both BM and MF samples consist of diverse morphologies such as classic lipoplexes, unilamellar, bilamellar, multilamellar and fused vesicles. For the BM sample (FIG. 52A), the white arrowhead shows small multilamellar lipid nanoparticles (i.e. onion ring like structure), white pentagon shows larger multilamellar lipid nanoparticles, and white arrow shows large unilamellar vesicles. For the MF sample (FIG. 52B), white arrowhead shows small multilamellar lipid nanoparticles (i.e. onion ring like structure), white pentagon shows larger multilamellar lipid nanoparticles, white arrow shows large unilamellar vesicles, and black arrow shows bilamellar vesicles. The MF LPs size distribution was on average smaller than BM LPs and was comprised of more bilamellar (black arrow) and small multilamellar lipid nanoparticles (white arrowhead). In general, BM and MF prepared LP nanoparticles have similar structures, although the aggregates size distribution might be a little smaller.

After production, the solution was dialyzed twice, filtered using 0.2 μm PVDF filter, and stored at 4° C. We tested both nylon and PVDF filters for sample sterilization and found that more than 90% of ODN was lost after filtering with the nylon filter as compared to approximately 20% of ODN lost when using the PVDF filter (data not shown).

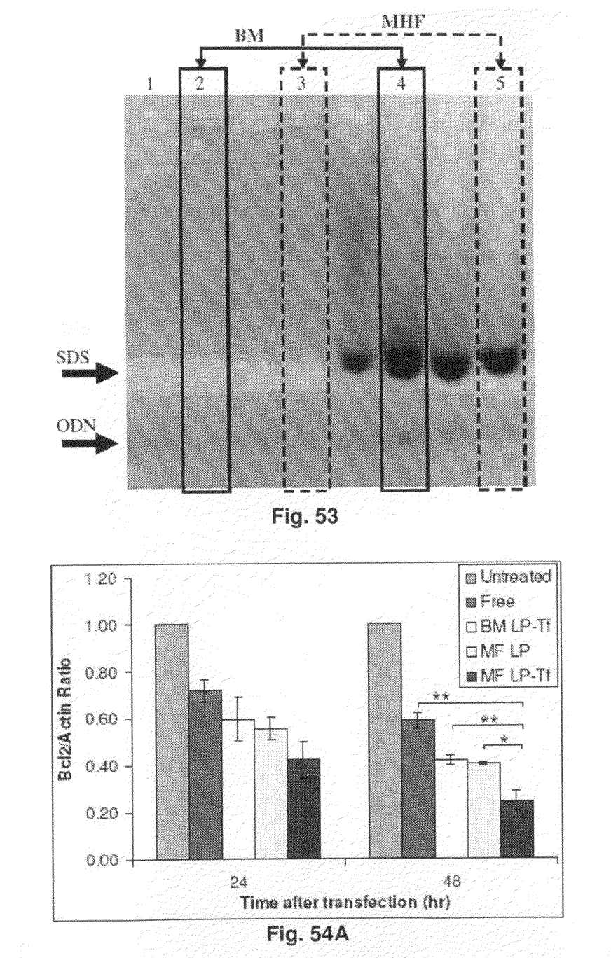

Analysis of ODN encapsulated in LPs. After LP nanoparticles production by BM and MF methods, the solutions were dialyzed twice, filtered using 0.2 μm PVDF filter, and stored at 4° C. In certain embodiments, the type of membrane material used for filtering and sterilizing the samples was important to retain ODN in the samples. We tested both nylon and PVDF filters for sample sterilization and found that more than 90% of ODN was lost after filtering with the nylon filter as compared to approximately 20% of ODN lost when using the PVDF filter (data not shown). After PVDF filtering, the ODN encapsulation efficiency of BM and MF produced LPs were analyzed by electrophoresis in 3% agarose gel at 100V for 45-60 min. As shown in FIG. 53, high encapsulation efficiencies at 94% and 92% for BM and MF, respectively, were obtained.

In vitro Bcl-2 downregulation. The effect of G3139 in the BM and MF LPs on downregulation of Bcl-2 at both protein and mRNA levels in K562 cells was evaluated by western blot and real-time RT-PCR, respectively. K562 cells were treated with free G3139, Tf conjugated G3139-containing lipid nanoparticles produced by BM (BM Tf-LP), non-targeted G3139-containing lipid nanoparticles produced by MF (MF LP), and Tf conjugated G3139-containing lipid nanoparticles produced by MF (MF Tf-LP). G3139 concentration in the free group was 1 μM in all experiments. From FIG. 54A, the densitometry analysis revealed that Bcl-2 protein levels 24 hr after transfection were decreased by 58%±8% by G3139 in MF Tf-LPs as compared to 28%±5% by free G3139, 44%±5% by G3139 in non-targeted MF LP, and 40%±9% by G3139 in BM Tf-LPs. In addition, Bcl-2 protein levels 48 hr after transfection were decreased by 75%±4% by G3139 in MF Tf-LPs as compared to 41%±3% (p<0.01) by free G3139, 59%±1% (p<0.05) by G3139 in non-targeted MF LP, and 58%±2% (p<0.01) by G3139 in BM Tf-LPs (FIG. 54A). Less than 10% Bcl-2 downregulation was observed following treatment with a mismatch ODN (data not shown).

From FIG. 54B, at 24 hr after transfection, the bcl-2 mRNA levels were decreased by 26%±2% by G3139 in MF Tf-LPs as compared to 13%±9% by free G3139, 12%±1% by G3139 in non-targeted MF LP, and 15%±2% by G3139 in BM Tf-LPs. In addition, at 48 hr after transfection, bcl-2 mRNA levels were decreased by 54%±4% by G3139 in MF Tf-LPs as compared to 18%±1% by G3139 in non-targeted MF LP, 55%±27% by G3139 in BM Tf-LPs, and for free G3139, the mRNA level was increased.

The effect of G3139 concentration in the Tf conjugated BM and MF LPs on downregulation of Bcl-2 protein level was also evaluated.

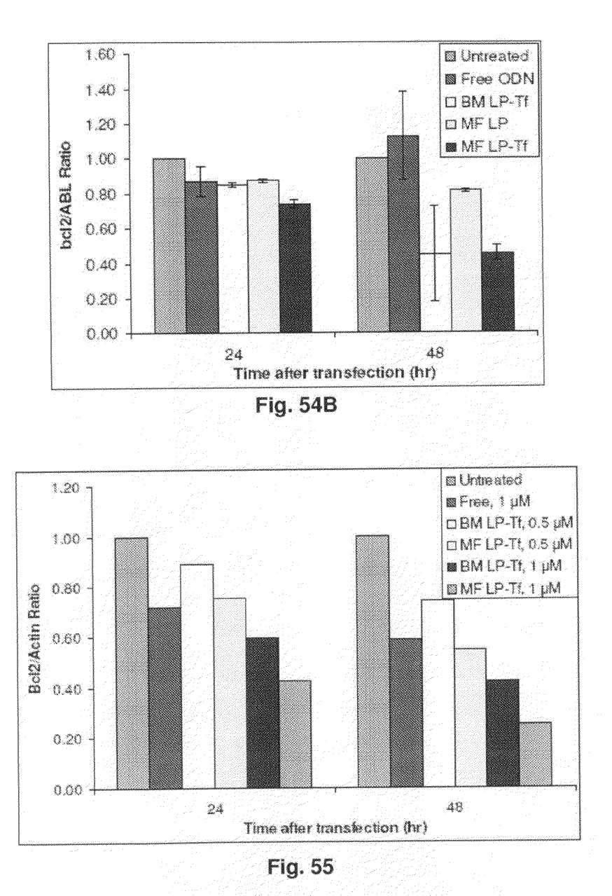

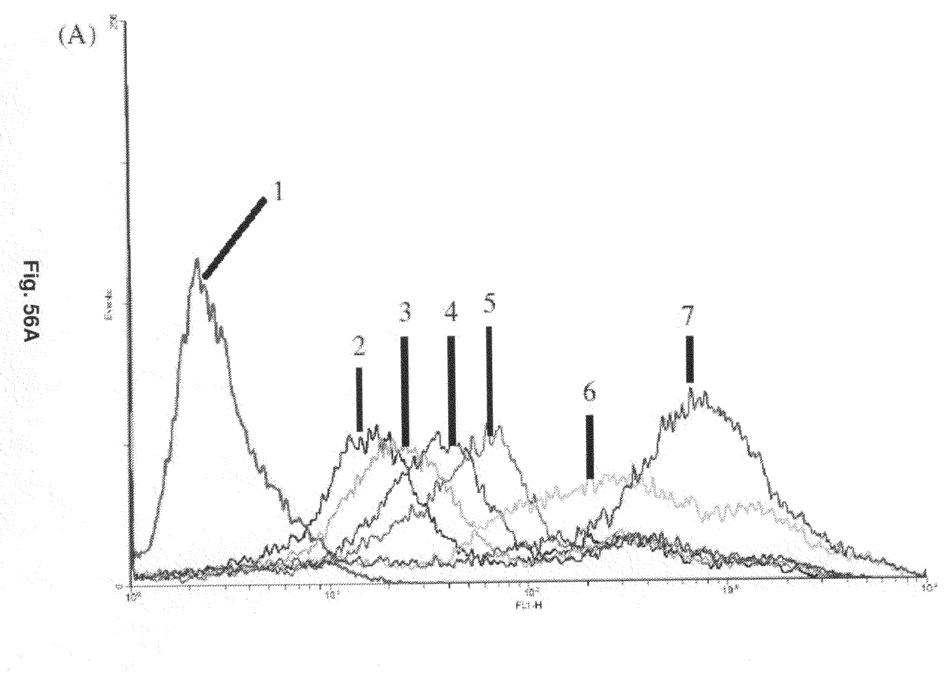

As shown in FIG. 55, the higher the amount of G3139 in the LPs, the better the downregulation of Bcl-2. In general, the MF Tf-LP downregulated Bcl-2 to a greater extend as compared to free G3139, non-targeted MF LP, and BM Tf-LP. In addition, the Bcl-2 downregulation by non-targeted MF LP containing only 0.5 μM was comparable to free G3139 (1 μM) which indicated that LP could deliver the ODN more efficiently into the cells even without transferrin targeting.

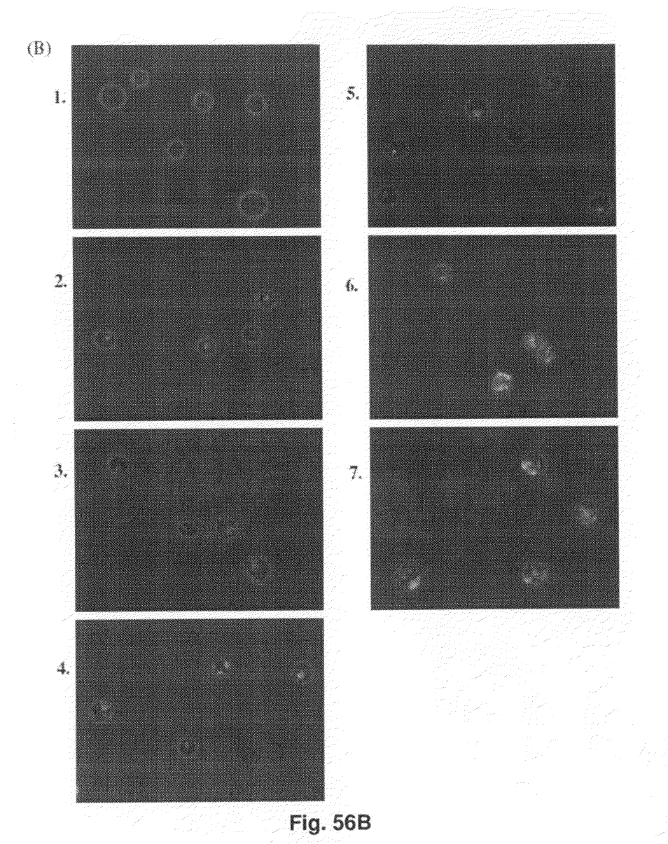

Cellular uptake of FITC-labeled G3139 analyzed with FCM. The relative uptake of LPs might play a significant role in Bcl-2 downregulation in the IC562 cells. Flow cytometry was used to analyze the uptake of non targeted and targeted LPs containing FITC-labeled G3139 produced by BM and MF methods as shown in FIGS. 56A-56B.

In FIG. 56A, 1 is untreated cell control, 2 is cells treated with non-targeted BM LP, 3 is cells treated with targeted BM Tf-LP, 4 and 6 are cells treated with non-targeted MF LP, and 5 and 7 are cells treated with targeted MF Tf-LP. Samples 2 to 5 were treated for 6 hr whereas 6 and 7 were treated for 24 hr. By comparing samples 2 and 3 or 4 and 5, we can see that with Tf targeting, more FITC-labeled G3139 were uptake into the cells, i.e., a shift of the curve to the right. By comparing 3 (BM Tf-LP) and 5 (MF Tf-LP), we can see that MF Tf-LP deliver more FITC-labeled G3139 into the cells than the BM Tf-LP. In addition, the MF LP (sample 4) was also more efficient in delivering FITC-labeled G3139 into the cells than the BM Tf-LP (sample 3). When the cells were treated for 24 hours, the distribution of BM Tf-LPs (sample 6) in the cells was over a broad range; conversely, the distribution of MF Tf-LP (sample 7) was narrower and more cells express higher fluorescence signal. The merged fluorescence images of the samples are shown in FIG. 56B. The brighter the fluorescence signal indicates more MC-labeled G3139 accumulation in the cells.

Induction of apoptosis by G3139 analyzed with FCM. For healthy cells, phosphatidylserine (PS) is located in the inner leaflet of the cell membrane. However, when cells are in the early apoptotic pathway, PS, translocates from the interior to the exterior of the cell membrane and can be recognized by Annexin V-FITC. The cells were simultaneously stained with viability dye propidium iodide (PI) where viable cells will exclude both the PI and the AV-FITC from the interior of the cell. In this analysis, the cell debris was excluded by gating the region believed to be containing cells in the Forward versus Side Scatter dot plot.

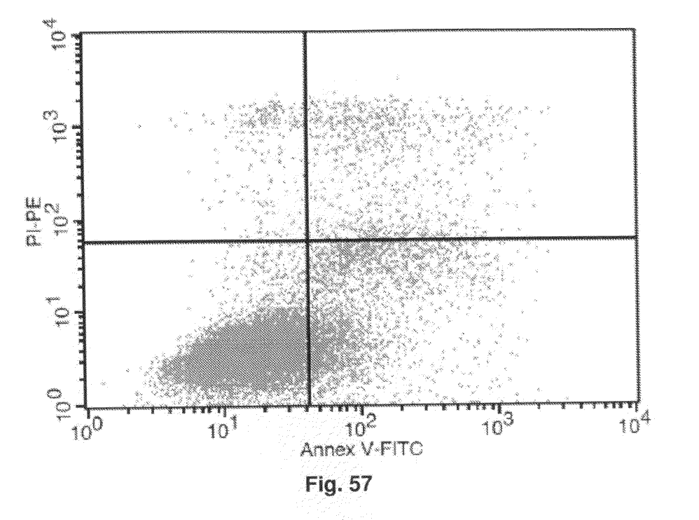

FIG. 57 shows a FCM bivariate plot of PI versus AV-FITC. The lower left (LL), lower right (LR), upper right (UR), and upper left (UL) quadrants correspond to cells that are negative for both dyes and are viable, positive only for AV-FITC which are cells in early stages of apoptosis and are viable, positive for both AV-FITC and PI which are cells in late stages of apoptosis or already dead, and positive for PI which are dead cells lacking membrane-based PS, respectively.

Table 10 shows the flow cytometry analysis of Annexin V-FITC stained k562 cells after treatment with G3139 and LP formulations. At 24 hr post transfection, the percentage of untreated control, free G3139, BM Tf-LP, MF LP, and MF Tf-LP treated cells in early stages of apoptosis were 18.1%, 25.5%, 9.7%, 6.0%, and 7.0%, respectively, and in late stages of apoptosis were 6.0%, 6.8%, 13.4%, 12.5%, and 19.5%, respectively. At 48 hr post transfection the percentage of cells in early stages of apoptosis were 24.1%, 18.0%, 18.4%, 12.3%, and 11.9%, and in late stages of apoptosis were 18.1%, 25.5%, 9.7%, 6.0%, and 7.0%, respectively.

TABLE 10
Flow cytometry analysis of Annexin V-FITC stained K562 cells
after treatment with free G3139 and different LP formulations.
Table 10. Flow cytometry analysis of Annexin V-FITC stained K562 cells
after treatment with free G3139 and different LP formulations

| Time after | | % Early | % Late | % Total |
|---|---|---|---|---|
| transfection (hr) | Sample | Apoptotic | Apoptotic | Apoptotic |
| 24 hrs | Untreated | 18.1 | 6.0 | 24.1 |
| | Free G3139 | 25.5 | 6.8 | 32.3 |
| | BM PL-Tf | 9.7 | 13.4 | 23.2 |
| | MF LP | 6.0 | 12.5 | 18.5 |
| | MF LP-Tf | 7.0 | 19.5 | 26.5 |
| 48 hrs | Untreated | 24.1 | 3.8 | 27.9 |
| | Free G3139 | 18.0 | 6.5 | 24.5 |
| | BM PL-Tf | 18.4 | 15.7 | 34.1 |
| | MF LP | 12.3 | 22.4 | 34.6 |
| | MF LP-Tf | 11.9 | 29.4 | 32.3 |

### Discussion of Example L

The 5-inlet polymeric microfluidic hydrodynamic focusing (MF) system is useful for producing lipid-polymer-DNA nanoparticles (lipopolyplex or LP) of controlled size, size distribution, and uniform morphology. The MF system can precisely control the flow conditions and mixing process of reagents at the micrometer scale by using syringe pumps to independently control the flow rate of the fluid streams. Since the Reynolds number in the microchannel is typically less than 1, the flow is strictly laminar which allows well-defined mixing to be controlled solely by interfacial diffusion between the multiple flow streams in a single microchannel. In certain embodiments, this is important since BM is a heterogeneous and uncontrolled chemical and/or mechanical process which can result in a heterogeneous population of LPs.

There are a few factors that govern the successful application of LPs in vitro and in vivo such as particle size and size distribution, surface charge or zeta potential, ODN encapsulation efficiency, colloidal stability, etc.

In Example L, the lipids used in the formulation included DC-Chol, egg PC, and PEG-DSPE. DC-Chol is a cationic lipid with a tertiary amine headgroup. This allows for assembly of LPs at pH 4, where DC-Chol is fully ionized, and reduction of positive charge of the LPs upon returning the pH to 7.4, where DC-Chol is partially deprotonated

The amount of cationic lipid (DC-Chol) was kept relatively low to produce a zeta potential close to zero. PEG-DSPE was added to the bilayer to reduce plasma protein binding and to provide enhanced particle colloidal stability. For targeting, transferrin (Tf) was used and incorporated into Tf-DSPE-PEG micelles for post insertion. Tf was an iron transport protein that, when associated with ferric ion binds with high affinity to transferrin receptor (TfR), which is overexpressed frequently on leukemia cells. Transferrin receptor (TfR) targeted lipoplexes have been shown to improve the delivery of G3139 to human erythroleukemia K562 cells, which overexpress TfR. Both the non-targeted and transferrin-receptor targeted nanoparticles carrying G3139 produced by BM (BM Tf-LP) and MF (MF Tf-LP) were applied to the K562 leukemia cells to evaluate efficacy of Bcl-2 downregulation.

We have characterized particle size and zeta potential of the nanoparticles prepared by the MF and BM methods. For the first flow configuration, the protamine binds to the ODN via electrostatic interaction between negatively charged ODN and positively charged protamine to form a compact ODN/protamine nanoparticles or “proticles”. The lipids streams which were introduced sequentially would then sandwich the proticles. However, since the proticles have a solid core and are negatively charge (−29.8 mV) at ODN/protamine of 1/0.3 (wt/wt), their sizes are dominated by their solid cores. In fact, increasing the flow rate of the lipids stream did not significantly decrease the size of the proticles even though; a higher FRR results in a narrower ODN/protamine streams width, i.e. a shorter diffusion length. Proticles have a size range of 100-300 nm when mixed in DI water, however, when mixed in sodium citrate buffer, proticles tend to aggregate almost instantly. Therefore, protamine was premixed with lipids before addition of the ODN solution.

As shown in FIG. 51, the MF LP nanoparticles were slightly smaller in size than the BM particles in all the processing steps. To confirm the smaller size, cryo-TEM was used to image the BM and MF samples in their frozen hydrated state. As shown in FIG. 53, both BM and MF LPs consist of diverse morphologies such as unilamellar, bilamellar, and multilamellar vesicles. The MF LPs size was on average smaller than BM LPs and was comprised of more bilamellar and small multilamellar lipid nanoparticles.

The surface charge (zeta potential) of the nanoparticles can influence the stability and cellular uptake of the nanoparticles. The zeta potential of the MF LP nanoparticles was also slightly lower than the BM particles probably due to more Tf-DSPE-PEG incorporation into the MF LPs. To enhance cellular uptake of LPs, the zeta potential is typically greater than 25 mV. Since moderate zeta potentials were obtained for both BM and MF Tf-LPs, this indicates that the enhance cellular uptake of the MF LP nanoparticles is due to their smaller size and size distribution in addition to the transferrin receptor (TfR) targeting.

The encapsulation efficiency of the two types of LPs was analyzed by electrophoresis in 3% agarose gel at 100V for 45 min. As shown in FIG. 53, high encapsulation efficiencies for both types of particles, at 94% and 92% for BM and MF, respectively, were obtained.

For targeting, transferrin (Tf) was used and incorporated into Tf-DSPE-PEG micelles for post insertion. Transferrin receptor (TfR) targeted lipopolyplexes (LPs) have been shown to improve the delivery of G3139 to human erythroleukemia K562 cells, which overexpress TfR.

In Example L, both the non-targeted and transferrin-receptor targeted nanoparticles carrying G3139 produced by BM (BM Tf-LP) and MF (MF Tf-LP) were applied to K562 leukemia cells. As shown in FIG. 54 and FIG. 55, MF Tf-LP nanoparticles were more effective than BM Tf-LP nanoparticles in Bcl-2 downregulation. Greater downregulation was observed in 48 hr than in 24 hr both BM and MF LP nanoparticles. This result is supported by flow cytometry analysis of FITC-labeled G3139 uptake by K562 cells as shown in FIG. 56 where more MF Tf-LPs were uptake as indicated by the higher fluorescence signal as compared BM Tf-LPs.

Apoptosis is the programmed cell death in the cell's life cycle. G3139 has been shown to enhance apoptosis, however, in Example L the percentage of cells undergoing apoptosis were similar between free, BM Tf-LP, MF LP, and MF Tf-LP treated cells. Therefore, apoptosis induced by G3139 might not have played a significant role in Bcl-2 downregulation. As such, Example L shows a novel 5-inlet MF system and produced LP nanoparticles with smaller size and size distribution, moderate zeta potential, and high ODN encapsulation efficiency. The MF G3139 Tf-LP nanoparticles exerted greater downregulation effect on Bcl-2 in K562 cells than the particles produced by the conventional BM method, indicating that MF produced LP improved ODN delivery via better size control during the particle assembly.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

Sequence Listings

G3139
[SEQ ID NO: 1]
(5′-TCT CCC AGC GTG CGC CAT-3′)
bc1-2 primers and probes
(forward primer
[SEQ ID NO: 2]
CCCTGTGGATGACTGAGTACCTG;
reverse primer
[SEQ ID NO: 3]
CCAGCCTCCGTTATCCTGG
probe
[SEQ ID NO: 4])
CCGGCACCTGCACACCTGGA.
control ODNs G4126
[SEQ ID NO: 5]
(5′-TCT CCC AGC ATG TGC CAT-3′)
(2 nucleotides different from G3139 and
containing no CpG motifs)
G3622
[SEQ ID NO: 6]
(5′-TAC CGC GTG CGA CCC TCT-3′)
and
a FAM-terminus modified ODN
[SEQ ID NO: 7]
(5′-(6)FAM-TAC CGC GTG CGA CCC TCT-3′),
GTI-2040
[SEQ ID NO: 8]
(sequence 5′-GGCTAAATCGCTCCACCAAG-3′),
ODN with scrambled sequence
[SEQ ID NO: 9]
(5′-ACGCACTCAGCTAGTGACAC-3′),
β-actin
Forward primer
[SEQ ID NO: 10]
5′-TCC CTG GAG AAG AGC TAC GA-3′
Reverse primer
[SEQ ID NO: 11]
5′-AGC ACT GTG TTG GCG TAC AG-3′
R1
Forward primer
[SEQ ID NO: 12]
5′-AAC AAG GTC GTG TCC GCA AA-3′
Reverse primer
[SEQ ID NO: 13]
5′-CAT CTT TGC TGG TGT ACT CC-3′

1. An oligonucleotide-lipid nanoparticle comprising at least one oligonucleotide, at least one lipid and at least one complexation agent for the oligonucleotide formed by:
i) mixing at least one lipid and at least one complexing agent and one or more cationic polymers, in a water miscible organic solvent to form a first mixture;
ii) dissolving one or mixing two or more oligonucleotides in an aqueous buffer to form a second mixture; and,
iii) injecting the first mixture into the second mixture, or mixing the first mixture and the second mixture under pressure, to form a third mixture; and
iv) removing the organic solvent from the third mixture to form the oligonucleotide-lipid nanoparticle.

2. An oligonucleotide-lipid nanoparticle comprising at least one oligonucleotide, at least one lipid and at least one complexation agent for the oligonucleotide formed by:
i) mixing at least one complexing agent and at least one oligonucleotide in an aqueous buffer to form a first mixture;
ii) dissolving at least one lipid in a water-miscible solvent to form a second mixture comprised of liposomes or liposome precursors;
iii) mixing the second mixture with the first mixture under pressure to form from a third mixture; and
iv) removing solvent from the third mixture to form the oligonucleotide-lipid nanoparticle.

3. (canceled)

4. The oligonucleotide-lipid nanoparticle of claim 1, wherein the complexing agent comprises one or more of: Ca²⁺, Mg²⁺, pentaethylenehexamine (PEHA), spermine, protamine, polylysine, chitosan, and polyethyleneimine (PEI).

5. (canceled)

6. (canceled)

7. The oligonucleotide-lipid nanoparticle of claim 1, further including at least one targeting ligand.

8. (canceled)

9. (canceled)

10. (canceled)

11. The oligonucleotide-lipid nanoparticle of claim 1, wherein the oligonucleotides contain one or more chemical modifications comprising one or more of a phosphorothioate linkages between the nucleotides, a cholesterol or lipid conjugated to the oligonucleotide at the 5′ or 3′ end, and 2′O-methylation on the ribose moieties.

12. The oligonucleotide-lipid nanoparticle of claim 1, wherein the lipid comprises one or more of: a) cationic or anionic lipids or surfactants; b) neutral lipids or surfactants; c) cholesterol; and d) PEGylated lipids or surfactants.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. The oligonucleotide-lipid nanoparticle of claim 7, wherein the targeting ligand comprises one or more of: transferrin, folate, oligosaccharides, and tissue or cell-specific antibodies, and is conjugated to a hydrophobic anchor comprising one or more of: phosphatidylethanolamine derivative, a lipophilic molecule, and cholesterol.

30. (canceled)

31. (canceled)

32. The method of claim 31, wherein in vivo circulation time is further extended by grafting one or more PEG polymers onto a surface of the oligonucleotide-lipid nanoparticle.

33. A method for protecting an oligonucleotide from degradation by nucleases and prolonging systemic circulation time in vivo, the method comprising
loading an oligonucleotide into a lipid nanoparticle, the oligonucleotide-lipid nanoparticle being formed by:
A) i) mixing at least one lipid and at least one complexing agent, including, but not limited to a divalent cation or one or more cationic polymers, in a water miscible organic solvent, with or without up to 50% water, to form a first mixture;
ii) mixing one or more oligonucleotides in an aqueous buffer to form a second mixture; and,
iii) injecting the first mixture into the second mixture or mixing the two under pressure to form a third mixture; and
iv) removing solvent from the third mixture to form the oligonucleotide-lipid nanoparticle; or,
B) i) mixing at least one complexing agent including, but not limited to a divalent cation or one or more cationic polymers, and at least one oligonucleotide in an aqueous buffer to form a first mixture;
ii) dissolving at least one lipid in a water miscible solvent containing 0-50% water to form a second mixture comprised of liposomes or a liposome precursor;
iii) mixing the second mixture with the first mixture under pressure to from a third mixture; and
iv) removing solvent from the third mixture to form the oligonucleotide-lipid nanoparticle.

34. (canceled)

35. (canceled)

36. (canceled)

37. (canceled)

38. (canceled)

39. (canceled)

40. (canceled)

41. The method of claim 33, further including one or more steps:
v) complexing or conjugating a targeting ligand to the oligonucleotide-lipid nanoparticle, or adding a lipid-conjugated targeting ligand followed by incubation;
vi) reducing the size of the oligonucleotide-lipid nanoparticles using one or more of sonication and high pressure homogenization;
vii) removing the oligonucleotide-lipid nanoparticles using tangential-flow diafiltration;
viii) sterilizing the lipid nanoparticles by filtration; and
ix) lyophilizing the oligonucleotide-lipid formulation in the presence of a lyoprotectant.

42. (canceled)

43. (canceled)

44. (canceled)

45. A method for delivering oligonucleotides to a subject in need thereof, the method comprising administering an effective amount of a therapeutic composition comprising one or more long-circulating oligonucleotide/lipid-nanoparticles, wherein the oligonucleotide/lipid-nanoparticle exhibits an enhanced permeability and retention (EPR) effect.

46. (canceled)

47. (canceled)

48. (canceled)

49. (canceled)

50. (canceled)

51. (canceled)

52. (canceled)

53. (canceled)

54. (canceled)

55. A method for making a microfluidic device, comprising:
laminating a film to form closed microchannels having inlets and outlets by passing a film sandwich through a thermal laminator;
sonicating the plates;
drying the plates; and
bonding fluidic connectors onto the inlets and outlet on the plate by applying a curing adhesive around a perimeter of each of the connectors,
wherein the connectors are aligned over inlet/outlet openings; and curing the adhesive.

56. A microfluidic device for making oligonucleotide-lipid nanoparticles, comprising at least three inlet ports and at least one outlet port,
each inlet port being connected to a separate injection device;
the device being configured such that:
i) when a first fluid stream is introduced into each of the first and second inlet ports, the first fluid stream is split into two side microchannel streams at the third inlet port; and
ii) when a second fluid stream is introduced in the third inlet port, a product stream is formed that is collected at the outlet port.

57. A microfluidic device for making oligonucleotide-lipid nanoparticles, comprising at least five inlet ports and at least one outlet port, each inlet port being connected to a separate injection device;
the device being configured such that:
i) when a first fluid stream is introduced into the first inlet port and a second fluid stream is introduced into the second inlet port, the first fluid stream is split into two side microchannel streams at the third inlet port;
ii) when a third fluid stream is introduced in the third inlet port, a first product stream is formed at a first junction;
iii) when a fourth fluid stream is introduced into the fourth inlet port and a fifth fluid stream is introduced into the fifth inlet port at a point downstream of the first junction, the fourth fluid stream and the fifth fluid stream contact the first product stream to form a second product stream at a second junction; the second product stream being collected at the outlet port.

58. (canceled)

59. A method of oligonucleotide-lipid nanoparticles, comprising:
i) introducing a first fluid stream into a first inlet port;
ii) introducing a second fluid stream into a second inlet port and a third fluid stream into a third inlet port, the second and third inlet ports being positioned on opposing sides of the first inlet port, the second and third fluid streams hydrodynamically focusing the first fluid stream into a narrow stream to form a first product stream at a first junction; and
iii) introducing downstream of the first junction a fourth fluid stream into a fourth inlet port and a fifth fluid stream into a fifth inlet port, the fourth and fifth inlet ports being positioned downstream to and on opposing sides of the first junction, the fourth and fifth fluid streams hydrodynamically focusing the first product stream into a narrow stream to form a second product stream.

60. The method of claim 59, wherein:
the first fluid stream comprises an oligonucleotide component;
the second fluid comprises a protamine sulfate stream;
the third fluid comprises a protamine sulfate stream;
the first product stream comprises oligonucleotide/protamine nanoparticles formed via electrostatic interaction between negatively charged oligonucleotides and positively charged protamine sulfate;
the fourth fluid stream comprises a lipid stream;
the fifth fluid stream comprises a lipid stream; and
the second product stream comprises oligonucleotide/protamine/lipids nanoparticles or lipopolyplexes.

61. (canceled)

62. (canceled)

63. (canceled)

64. The method of claim 59, wherein:
the first fluid stream comprises a protamine/lipids mixture stream;
the second fluid comprises a first oligonucleotide stream;
the third fluid comprises a second oligonucleotide stream;
the first product stream comprises an oligonucleotide/protamine/lipids stream;
the fourth fluid stream comprises a protamine/lipids stream;
the fifth fluid stream comprises a protamine/lipids stream; and
the second product stream comprises oligonucleotide/protamine/lipids nanoparticles.

65. (canceled)

66. (canceled)

67. (canceled)

68. (canceled)

69. (canceled)

70. (canceled)

71. (canceled)

72. The oligonucleotide-lipid nanoparticle of claim 2, wherein the complexing agent comprises one or more of: Ca²⁺, Mg²⁺, pentaethylenehexamine (PEHA), spermine, protamine, polylysine, chitosan, and polyethyleneimine (PEI).

73. The oligonucleotide-lipid nanoparticle of claim 2, further including at least one targeting ligand.

74. The oligonucleotide-lipid nanoparticle of claim 2, wherein the oligonucleotides contain one or more chemical modifications comprising one or more of a phosphorothioate linkages between the nucleotides, a cholesterol or lipid conjugated to the oligonucleotide at the 5′ or 3′ end, and 2′O-methylation on the ribose moieties.

75. The oligonucleotide-lipid nanoparticle of claim 2, wherein the lipid comprises one or more of: a) cationic or anionic lipids or surfactants; b) neutral lipids or surfactants; c) cholesterol; and d) PEGylated lipids or surfactants.

76. The oligonucleotide-lipid nanoparticle of claim 73, wherein the targeting ligand comprises one or more of: transferrin, folate, oligosaccharides, and tissue or cell-specific antibodies, and is conjugated to a hydrophobic anchor comprising one or more of: phosphatidylethanolamine derivative, a lipophilic molecule, and cholesterol.

77. A therapeutic composition, comprising an effective amount of lipid nanoparticles having incorporated therein one or more oligonucleotides and one or more complexing agents; and, having conjugated thereon one or more targeting ligands.

78. The composition of claim 77, wherein the oligonucleotide comprises G3139, an 18-mer phosphorothioate oligonucleotide targeting the anti-apoptotic protein Bcl-2, and the lipid nanoparticles comprise transferrin receptor (TfR)-targeted, protamine-containing lipid nanoparticles.

79. The composition of claim 78, further including DC-Chol as a cationic lipid and PEG-DSPE is incorporated into the oligonucleotide-lipid-nanoparticles.

80. A therapeutic composition for treatment of chronic lymphocytic leukemia (CLL), comprising:
an effective amount of the composition of claim 77, wherein the composition comprises an anti-CD20 antibody conjugated on lipid-nanoparticles carrying Bcl-2 targeted anti-sense oligonucleotides; and,
optionally, further including at least one of: vincristine and herceptin.

81. A therapeutic composition for delivering Mcl-1 siRNAs to a subject in need thereof, comprising:
an effective amount of the composition of claim 77, wherein the composition comprises anti-CD37 mAb conjugated lipid-nanoparticles; and,
optionally further including one or more of: fludarabine, chlorambucil, trastuzumab (Herceptin®), rituximab (Rituxan®), alemtuzumab (Campath®), formiversen, anti-CD20 and anti-CD19.

82. A therapeutic agent for overcoming chemoresistance in acute myeloid leukemia (AML), comprising:
an effective amount of the composition of claim 77, wherein the composition comprises GTI-2040 transferrin (TO conjugated pH-sensitive lipid-nanoparticles,
wherein GTI-2020 comprises an antisense oligodeoxyribonucleotide (ODN) against the R2 subunit of ribonucleotide reductase.

83. A therapeutic agent for reducing R1 gene expression, comprising:
an effective amount of the composition of claim 77, wherein the composition comprises GTI-2501 lipid-nanoparticles,
wherein CTI-2501 comprises a 20-mer oligonucleotide that is complementary to a coding region in the mRNA of R1, the large subunit of ribonucleotide reductase (RNR); and,
optionally, wherein the targeting ligand comprises holo-transferrin.

84. A method for ameliorating chemoresistance in a subject in need thereof, comprising: administering an effective amount of oligonucleotide-lipid nanoparticles of claim 77.

85. A method for ameliorating chemoresistance in a subject in need thereof, comprising: administering an effective amount of oligonucleotide-lipid nanoparticles of claim 78.

86. A method for restore chemosensitivity in leukemia cells, comprising:
administering an effective amount of oligonucleotide-lipid nanoparticles of claim 77; and, optionally,
enhancing delivery efficiency thereof by administering an effective amount of deferoxamine sufficient to up-regulate TfR expression on leukemia cells.

87. A method for restore chemosensitivity in leukemia cells, comprising:
administering an effective amount of oligonucleotide-lipid nanoparticles of claim 78; and, optionally,
enhancing delivery efficiency thereof by administering an effective amount of deferoxamine sufficient to up-regulate TfR expression on leukemia cells.

88. A method for increasing the anti-tumor activity of rituximab in a subject having CLL, comprising
administering an effective amount of the composition of claim 77 sufficient to achieve high transfection efficiencies and good targeting specificity to bind to B cell surfaces but not T cells.

89. A method for increasing the anti-tumor activity of rituximab in a subject having CLL, comprising
administering an effective amount of the composition of claim 78 sufficient to achieve high transfection efficiencies and good targeting specificity to bind to B cell surfaces but not T cells.

90. A method for increasing serum levels of one or more of IL-6 and IFN-γ in a subject in need thereof, comprising:
administering an effective amount of the composition of claim 77.

91. A method for increasing serum levels of one or more of IL-6 and IFN-γ in a subject in need thereof, comprising:
administering an effective amount of the composition of claim 78.

92. A method for promoting proliferation of natural killer (NK) cells and dendritic cells (DCs) in a subject in need thereof, comprising:
administering an effective amount of the composition of claim 77.

93. A method for promoting proliferation of natural killer (NK) cells and dendritic cells (DCs) in a subject in need thereof, comprising:
administering an effective amount of the composition of claim 78.

94. A method for preparing Tf-conjugated G3139-containing lipid nanoparticles of claim 80, comprising:
i) dissolving a lipid mixture egg PC/DC-Chol/PEG₂₀₀₀-DSPE in ethanol (EtOH);
ii) mixing the lipid mixture of step i) with protamine in a citrate buffer at ratios for lipid:protamine of about 12.5:0.3 (w/w) and EtOH:water of about 2:1 (v/v);
iii) dissolving G3139 in citrate buffer, and adding into the lipid/protamine mixture of step ii) to form pre-lipid-nanoparticle complexes at an EtOH concentration of 40% (v/v);
iv) dialyzing the pre-lipid-nanoparticle complexes of step iii) against citrate buffer, and then against HEPES-buffered saline to remove free G3139 and to form -G3139-lipid-nanoparticles, and adjusting the pH to the physiological range; and
v) incorporating, using a post-insertion method, Tf ligand into the G3139-lipid-nanoparticles.
